SEQUENCE LISTING (20 sequences)

SEQ ID NO: 1 - DNA, 20 bp, Artificial Sequence
  18S rRNA siRNA
caattacagggcctcgaaag

SEQ ID NO: 2 - DNA, 20 bp, Artificial Sequence
  18S rRNA siRNA
aaacggctaccacatccaag

SEQ ID NO: 3 - DNA, 20 bp, Artificial Sequence
  hAtf4 siRNA
tcccatctccaggtgttctc

SEQ ID NO: 4 - DNA, 20 bp, Artificial Sequence
  hAtf4 siRNA
cagctctttgcactcaccag

SEQ ID NO: 5 - DNA, 20 bp, Artificial Sequence
  hChop siRNA
gcgcatgaaggagaaagaac

SEQ ID NO: 6 - DNA, 21 bp, Artificial Sequence
  hChop siRNA
aaagctgcgcagaatgagatg

SEQ ID NO: 7 - DNA, 23 bp, Artificial Sequence
  hIl6 siRNA
agttcctgcagaaaaaggcaaag

SEQ ID NO: 8 - DNA, 21 bp, Artificial Sequence
  hIL6 siRNA
aaagctgcgcagaatgagatg

SEQ ID NO: 9 - DNA, 25 bp, Artificial Sequence
  Gcn2 Mutant reverse Primer
tgccactgtcagaatctgaagcagg

SEQ ID NO: 10 - DNA, 24 bp, Artificial Sequence
  Gcn2 Forward primer
tctcccagcggaatccgcacatcg

SEQ ID NO: 11 - DNA, 24 bp, Artificial Sequence
  Gcn2 Reverse Primer
atccaggcgttgtagtagcgcaca

SEQ ID NO: 12 - DNA, 22 bp, Artificial Sequence
  Il6 Common forward Primer
ttccatccagttgccttcttgg

SEQ ID NO: 13 - DNA, 23 bp, Artificial Sequence
  Il6 WT reverse Primer
ttctcatttccacgatttcccag

SEQ ID NO: 14 - DNA, 22 bp, Artificial Sequence
  Il6 Mutant reverse Primer
ccggagaacctgcgtgcaatcc

SEQ ID NO: 15 - DNA, 25 bp, Artificial Sequence
  Ifn-gamma WT forward Primer
agaagtaagtggaagggcccagaag

SEQ ID NO: 16 - DNA, 21 bp, Artificial Sequence
  Inf-gamma Mutant forward Primer
ccttctatcgccttcttgacg

SEQ ID NO: 17 - DNA, 25 bp, Artificial Sequence
  Ifn-gamma Common reverse Primer
agggaaactgggagaggagaaatat

SEQ ID NO: 18 - DNA, 18 bp, Artificial Sequence
  IDO1 Common Forward Primer
ggtggagctgcccgacgc

SEQ ID NO: 19 - DNA, 20 bp, Artificial Sequence
  IDO1 WT reverse Primer
ttccaccaccaccgagttac

SEQ ID NO: 20 - DNA, 20 bp, Artificial Sequence
  IDO1 Mutant reverse Primer
aatgcgctcaggtcaaattc

(19) United States
(12) Patent Application Publication
(10) Pub. No.: US 2024/0084002 A1
(43) Pub. Date: Mar. 14, 2024

(54) METHODS AND COMPOSITIONS UTILIZING IDO1-DEPENDENT VASCULARIZING CELLS FOR THE TREATMENT OF PATHOLOGICAL CONDITIONS INVOLVING NEOVASCULARIZATION

(71) Applicants: LANKENAU INSTITUTE FOR MEDICAL RESEARCH, Wynnewood, PA (US); DUET THERAPEUTICS, INC., Skillman, NJ (US)

(72) Inventors: Alexander J. Muller, Swarthmore, PA (US); Arpita Mondal, Havertown, PA (US); Souvik Dey, Havertown, PA (US); Simon Tomlinson, Skillman, NJ (US); Lisa Laury-Kleintop, Chesterbrook, PA (US)

(73) Assignees: LANKENAU INSTITUTE FOR MEDICAL RESEARCH, Wynnewood, PA (US); DUET THERAPEUTICS, INC., Skillman, NJ (US)

(21) Appl. No.: 18/265,774

(22) PCT Filed: Dec. 7, 2021
(86) PCT No.: PCT/US2021/062217

§ 371 Date: Jun. 7, 2023

Related U.S. Application Data
(60) Provisional application No. 63/122,121, filed on Dec. 7, 2020.

(51) Int. Cl. C07K 16/28; A61K 39/395; A61K 45/06; A61P 27/02; C07K 16/24; C12N 15/113

(52) U.S. Cl. CPC C07K 16/2803; A61K 39/3955; A61K 45/06; A61P 27/02; C07K 16/247; C07K 16/2812; C07K 16/2827; C07K 16/2845; C07K 16/2851; C07K 16/2863; C07K 16/2896; C12N 15/1137; C12N 2310/14; C12N 2310/531

(57) ABSTRACT

Compositions and methods for treating a retinopathy or inhibiting pathologic neovascularization in a subject are provided herein. In one embodiment, the method includes ablating or inhibiting IDO-dependent vascularizing cells (IDVCs) in the eye of the subject. Various methods of inhibiting or ablating IDVCs are described, including inhibiting IDO1 and/or integrated response nodes.

### CROSS-REFERENCE TO RELATED APPLICATIONS

This application claims priority to U.S. Provisional Application 63/122,121 filed Dec. 7, 2020, the entire disclosure being incorporated herein by reference.

### INCORPORATION-BY-REFERENCE OF MATERIAL SUBMITTED IN ELECTRONIC FORM

Incorporated herein by reference in its entirety is the sequence listing submitted via EFS-Web as a text file named SEQLIST.txt, created Dec. 7, 2021, and having a size of 4,212 bytes.

### BACKGROUND OF THE INVENTION

Angiogenesis, the tightly regulated process of new blood vessel formation, is developmentally required for organogenesis but otherwise rare in normal adult tissues except in the female reproductive cycle and in wound healing. Pathological angiogenesis, by contrast, is marked by dysregulated neovascularization and contributes to a variety of diseases including the development of tumors, which require a blood supply from the host in order to grow beyond a certain stage (1). Tumor angiogenesis is elicited through interaction with the local inflammatory environment that subverts the physiological process of wound healing (2). One distinguishing feature of inflammatory tumor microenvironments is the presence of a heterogeneous assortment of immature myeloid cells referred to collectively as MDSCs (myeloid-derived suppressor cells) (3). In addition to MDSCs' hallmark immunosuppressive capability, the ability to promote angiogenesis is currently regarded as a defining characteristic, and MDSCs are considered to be important drivers of tumor neovascularization (4).

IFNγ and IL6 are two key inflammatory cytokines that appear to function antagonistically with regard to curbing or promoting angiogenesis respectively (5,6). The biological ramifications of these apparently antagonistic activities have been unclear, although recent genetic data implicate the tryptophan catabolizing enzyme IDO1 (indoleamine 2,3-dioxygenase 1) as a key regulatory node that sustains neovascularization by responding to local IFNγ and counterbalances its anti-angiogenic activity by signaling for increased IL6 production (7). IFNγ's ability to induce IDO1 has been extensively characterized (8), however, the regulatory connection between IDO1 and IL6 has been poorly understood, complicated by apparently contradictory previously published findings in which IDO1 has been found to exert either a positive or a negative impact on IL6 induction (9-12). Even with regard to IDO1 being a positive driver of IL6 induction, at least two distinct signaling pathways have been implicated. In some studies, the induction of IL6 by IDO1 has been attributed to signaling through the ISR (integrated stress response) pathway following activation of the GCN2 (general control nonderepressible 2) serine kinase by IDO1-mediated tryptophan depletion (11), while other studies have implicated AHR (aryl hydrocarbon receptor) signaling in response to IDO1-initiated production of the endogenous AHR ligand kynurenine (13). One possible explanation for these discrepancies is context, namely the relevant cell type in which IDO1 is expressed. IDO1 expression has been reported in a variety of immune and non-immune cells including MDSCs as well as DCs (dendritic cells), macrophages, NK (natural killer) cells, endothelial cells, mesenchymal stromal cells and fibroblasts (14). While the genetic evidence strongly implicates IL6 as a downstream effector required for IDO1 to promote neovascularization, because the cell type in which IDO1 is expressed in the context of neovascularization has yet to be identified, the underlying signaling pathway responsible for the IDO1 to IL6 connection also has yet to be resolved.

### SUMMARY OF THE INVENTION

Provided herein, in one aspect, is a method of treating a retinopathy or inhibiting pathologic neovascularization in a subject. The method includes ablating or inhibiting IDO-dependent vascularizing cells (IDVCs) in the eye of the subject. The IDVCs are functionally characterized as having a role in neovascularization the establishment and maintenance of requires the induction of IDO1 within the IDVCs. In one embodiment, the inhibition of IDVC activity is achieved by inhibition of IDO1. In another embodiment, the IDVCs are inhibited or ablated using an antibody directed to a cell-surface marker of the IDVCs, or an antibody-drug conjugate (ADC). In another embodiment, the IDVCs are inhibited or ablated by blocking or inhibiting the Integrated Stress Response (ISR) in IDVCs.

In one embodiment, the method includes (a) blocking or inhibiting the expression, induction, activity, or signaling of GCN2; and/or (b) blocking or inhibiting the expression, induction, activity, or signaling of ATF4; and/or (c) blocking or inhibiting the expression, induction, activity, or signaling of CHOP.

In another aspect, a method includes blocking or inhibiting signaling molecules downstream of the Integrated Stress Response. In one embodiment, the signaling molecule is a cytokine. In another embodiment, the cytokine is IL-6.

In another embodiment of the methods described herein, the method further includes blocking or inhibiting the expression, induction, activity, or signaling of any form of vascular endothelial growth factor (VEGF). In another embodiment, the method further includes administering an inhibitor of the expression, induction, activity, or signaling of indoleamine 2,3 dioxygenase-1 (IDO1).

Therapeutic compositions comprising the therapies, including kits for administration and other aspects and advantages are described in the detailed description below.

### BRIEF DESCRIPTION OF THE DRAWINGS

FIG. 1A-1D shows loss of GCN2 phenocopies loss of IDO1 in restricting 4T1 lung metastasis outgrowth, neovascularization and IL6 elevation. (FIG. 1A) Staining of lungs with India ink to visualize metastatic burden at 5 weeks following orthotopic 4T1 mammary tumor cell engraftment into WT, Ido1^{-/-}, and Gcn2^{-/-} mice. (FIG. 1B) Kaplan-Meier survival curves for cohorts of WT, Ido1^{-/-}, and Gcn2^{-/-} mice following orthotopic engraftment of 1×10⁴ 4T1 cells (N≥7 mice/group) with significance assessed by 2-group log-rank test. (FIG. 1C) Confocal images of immunofluorescent stained blood vessels (anti-CAV1; Cy3) and of nuclei (DAPI) within 4T1 lung metastases at 5-6 weeks from WT, Ido1^{-/-}, and Gcn2^{-/-} mice. Scale bars=100 μm. (FIG. 1D) Quantitative assessment of neovascular density within lung metastases from WT, Ido1^{-/-}, and Gcn2^{-/-} mice 5-6 weeks following orthotopic 4T1 mammary tumor cell engraftment (N=3 mice/group). Graphed as means±SEM with significance between the WT control group and the Ido1^{-/-} and Gcn2^{-/-} groups (indicated along the straight bar) determined by one-way ANOVA with Dunnett's multiple comparisons test, and significance between the Ido1^{-/-}, and Gcn2^{-/-} groups (indicated on bracket) determined by one-way unpaired Student's t-test.

FIG. 2A-2C demonstrate inhibition of ISR but not AHR signaling similarly blocks induction of IL6 expression as inhibition of IDO1 activity in vitro. (FIG. 2A) Spectrophotometric-based determination of kynurenine levels in the supernatants of IFNγ+LPS treated U937 and HL60 cells to assess the impact of IDO1 inhibitor (Epacadostat; left column), ISR inhibitor (ISRIB; middle column) and AHR inhibitor (BAY-218; right column) treatment on IDO1 activity. Fold induction relative to controls without IFNγ+LPS induction is graphed as the mean±SEM (N=3 trials/condition). (FIG. 2B-2C) qPCR-based assessment of IFNγ+LPS mediated induction (by row) of I16, Atf4, Chop and Cyp1a1 gene expression over untreated baseline levels in U937 and HL60 cells, and the impact of IDO1 inhibitor (Epacadostat; left column), ISR inhibitor (ISRIB; middle column), and AHR inhibitor (BAY-218; right column) treatments on the respective induction of these genes. Fold induction relative to controls without IFNγ+LPS induction is graphed as the mean±SEM (N=3 trials/condition). Statistical significance of the effects of the different inhibitor treatments was determined by two-tailed Student's t-test. Percent inhibition due to inhibitor treatments is also noted at the top of each graph.

FIG. 3A-3G demonstrate blocking ISR signaling in oxygen-induced retinopathy limits neovascularization and IL6 induction while IDO1 remains elevated. (FIG. 3A) Fluorescence microscopy of vasculature in retinal flatmounts following OIR and normoxic conditions. (FIG. 3B-FIG. 3D) Eyes from WT, Ido1^{-/-} and Gcn2^{-/-} OIR cohorts assessed for at P17 of (FIG. 3B) neovascular area over total retinal area (N≥6 eyes/group), (FIG. 3C) IL6 present in pooled vitreous humors (40 eyes/pool; N≥2 pools/group), (FIG. 3D) kynurenine present in pooled vitreous humors (40 eyes/pool; N≥2 pools/group). (FIG. 3B-3D) Straight bar: ANOVA with Dunnett's test comparing WT to other groups. (FIG. 3E) WT OIR cohorts injected intraocularly with siRNAs targeting Gcn2, Atf4, Chop, Ahr or the Non-Targeted Control assessed for neovascular area over total retinal area (N>eyes/group) with t-tests. (FIG. 3F, 3G) Confocal images of retinal flatmounts (from FIG. 3B and 3E) stained for blood vessels (B4-Alexa 488-Isolectin) and IDO1 (CY3). Scale bars=50 μm.

FIGS. 4A-4E demonstrate localization of IDO1 expression to a Gr1-positive immune cell population in 4T1 metastases and OIR. Confocal images stained for: (FIG. 4A) IDO1 (Cy3) and blood vessels (anti-CAV1; FITC) from WT, IDO1^{-/-}, and Gcn2^{-/-} (Scale bars=20 μm), (FIG. 4B) IDO1 (Cy3) and CD45 (FITC) from WT and IDO1^{-/-} mice compared with primary tumor and spleen from WT mice (Scale bars=50 μm), (FIG. 4C) IDO1 (Cy3) and Cr1 or CD11b (Alexa 488) from WT and IDO1^{-/-} mice (Scale bars=50 μm). (FIG. 4D) Retinal flatmounts stained for IDO1 (Cy3) and CD45 or Gr1 (FITC) from WT OIR-elicited neonates. (Scale bars=10 μm). (FIG. 4E) Neovascular area over total retinal area comparison between α-Gr1 and isotype control antibody injected WT OIR-elicited neonates (N≥eyes/group) with t-test.

FIGS. 5A-5F demonstrate Gr1+CD11b^{lo} subpopulation promotes neovascularization through expression of IDO1. (FIG. 5A) Flow cytometry plot of CD45+ magnetic bead selected, Gr1+ gated immune cells isolated from 4T1 metastasis burdened lungs showing gating for separation of CD11b^{lo} (P3) and CD11b^{hi} (P4) cells (for entire gating scheme see FIG. 12). (FIG. 5B) Confocal images of isolated CD45+ Gr1+ CD11b^{lo} and CD45+ Gr1+ CD11b^{hi} cells adhered to slides and stained for IDO1 (Cy3), CD11b (Alexa 488) and nuclei (DAPI). Scale bars=50 μm. (FIG. 5C, FIG. 5E) Matrigel photographs paired with confocal images stained for blood vessels (anti-CAV1; Cy3) and nuclei (DAPI). Matrigel plugs were incorporated with PBS alone or with 1.5×10⁶ Gr1+ CD11b^{lo} or Gr1+ CD11b^{hi} cells obtained: (FIG. 5C) from WT or Ido1^{-/-} mice and implanted into WT mice or (FIG. 5E) from WT mice and implanted into WT mice over the final 3 days received vehicle or 50 mg/kg epacadostat b.i.d.. Scale bars=100 μm. (FIG. 5D, 5F) Neovascular density quantitation corresponding to images in FIG. 5C and 5E (N≥5 plugs/condition). Straight bar: ANOVA with Dunnett's test for PBS compared with other groups. Brackets: ANOVA with Sidak's test for selected pairs.

FIGS. 6A-6F show that to promote neovascularization, Gr1+ CD11b^{lo} cells require both IL6 and GCN2 and counteract IFNγ by inducing IDO1. (FIG. 6A, 6E) Matrigel plug photographs paired with confocal images stained for blood vessels (anti-CAV1; Cy3) and nuclei (DAPI). Matrigel plugs were incorporated with PBS alone or with 1.5×10⁶ Gr1⁺ CD11b^{lo} or GR1⁺ CD11b^{hi} cells obtained: (FIG. 6A) from Il6^{-/-} or Gcn2^{-/-} mice and implanted into WT mice or (FIG. 6E) from WT or Ido1^{-/-} mice and implanted into Ifng^{-/-} mice. Scale=100 μm. (FIG. 6B, 6F) Quantitative assessment of neovascular density corresponding to images in FIG. 6A and FIG. 6E (N≥3 plugs/condition). Straight bar: ANOVA with Dunnett's test for PBS compared with other groups. Brackets: ANOVA with Sidak's test for selected pairs. (FIG. 6C, 6D) Confocal images obtained from WT and Ifng mice of: FIG. 6C retinal OIR flatmounts stained for blood vessels (B4-Alexa 488-Isolectin) and IDO1 (Cy3), or (FIG. 6D) 4T1 lung metastases for blood vessels (anti-CAV1: FITC) and IDO1 (Cy3). Scale=(FIG. 6C) 50 μm or (FIG. 6D) 10 μm.

FIGS. 7A-7F show that the autofluorescent subpopulation of Gr1+ CD11b^{lo} cells that promotes neovascularization includes an IDO1-dependent subset marked by high CD11c and asialo-GM1. (FIG. 7A) Confocal images of autofluorescence high (AF^{hi}) and low (AF^{lo}) subsets of Gr1⁺ CD11b^{lo} cells from 4T1 lung metastases stained for IDO1 (Cy3) and nuclei (DAPI). Scale=100 μm. (FIG. 7B) Matrigel plug photographs paired with confocal images (scale=100 μm) stained for blood vessels (anti-CAV1; Cy3) and DAPI. Matrigel plugs were incorporated with 1.5×10⁶ AF^{hi} or AF^{lo} cells isolated from and implanted into WT mice. (FIG. 7C) Plots following Gr1⁺ CD11b^{lo} gating: (left) gating on 488/530 intensity, and (right) subsequent gating on CD11c-PE (Y-axis) and asialo-GM1-APC intensity (x-axis) intensity. (FIG. 7D) CD1c^{hi} asialo-GM1^{hi} and CD11c^{lo} asialo-GM1^{lo} cells isolated as shown in FIG. 7C and stained: (left panels) for IDO1 (Cy3) and DAPI for confocal imaging (scale=50 μm), or (right panels) with Giemsa for light microscopy (scale=20 μm). (FIG. 7E) Quantitative assessment of neovascular density in Matrigel plugs incorporated with PBS alone or with 5×10⁴ CD11c^{hi} asialo-GM1^{hi} (hi/hi) or CD11c^{lo} asialo-GM1^{lo} (lo/lo) cells obtained from (left) WT or IDO1^{-/-} mice and implanted into WT mice or (right) WT, IDO1^{-/-}, Il61^{-/-}, or Gcn2^{-/-} mice and implanted into either WT or Ifng^{-/-} mice (N≥plugs/condition). The WT and IDO1^{-/-} to WT groups are included in both panels for comparative purposes. Straight bar: ANOVA with Dunnett's test for comparison of (left) PBS or (right) WT with other groups. Brackets: ANOVA with Sidak's test for selected pairs.

FIG. 8A-8B shows validation of transgenic mouse strains. (FIG. 8A) Genotyping analyses. Purified genomic DNA obtained from each of the four transgenic mouse strains utilized in this study and a wild type control were assayed by PCR with primer pairs designed to detect each of the genetic alterations resulting in functional disruption of the target gene (see Tables 1-3). The primers used for each set of analyses are indicated at the top of each gel image and the order in which the PCR products were loaded is listed on the side. Changes in the banding patterns for each transgenic stain conform with expectations for each set of primers. (FIG. 8B) Cytokine analyses. Lung homogenates from (left) Il6^{-/-} and (right) Ifng^{-/-} strain mice and corresponding WT controls were evaluated for IL6 and IFNγ levels respectively by cytokine bead array immunoassay-based analysis (BD Biosciences) as previously described (Smith, 2012, ref 9) at 24 hours following intranasal instillation of 25 μg LPS. Means±SEM (N=4 mice) are plotted on a log scale due to the large differential between the values obtained from the WT and knockout animals in each pairing.

FIG. 9. Primary 4T1 tumors exhibit comparable growth rates in WT, Ido1^{-/-} and Gcn2^{-/-} mice. Female WT, Ido1^{-/-} and Gcn2^{-/-} mice each received an orthotopic graft of 1×10⁴ 4T1 cells (N=5 tumors). Beginning at 11 days post-engraftment, when a palpable tumor mass had become apparent, caliper measurements were made on a bi-weekly basis to calculate primary tumor volumes. The data are plotted as means±SEM.

FIG. 10. Assessment of cell viability in response to various inhibitor treatments. Viability of U937 and HL60 cells induced with IFNγ+LPS and treated with either the IDO1 inhibitor Epacadostat, the ISR inhibitor ISRIB or the AHR inhibitor BAY-218 (top to bottom) was assessed using the LDI Cell CountEZ TPS assay kit. The percent cell survival for the various treatment groups was determined by normalizing to a corresponding set of untreated control cells and graphed as the mean±SEM (N=3 wells/condition). An additional set of cells in each group was treated with the proteasome inhibitor bortezomib (BTZ, 20 nM) as a positive control for pharmacologically induced cell death.

FIG. 11. Comparison of retinal flatmount images to visualize the impact on neovascularization of Gcn2 loss relative to Ido1 loss in a mouse model of oxygen-induced retinopathy. Representative images of B4-Alexa488-isolectin staining of blood vessels in retinal flatmounts obtained at P17 from: (top row) WT neonates exposed to hyperoxia from P7-P12 to induce OIR or maintained under constant normoxic conditions as an example of normal retinal vascularization, (bottom row) Ido1^{-/-} and Gcn2^{-/-} neonates exposed to hyperoxia from P7-P12 to induce OIR.

FIG. 12. Isolation of cells by flow cytometry for evaluation of blood vessel promotion using the surface markers Gr-1 and CD11b. Fluorescence-activated cell sorting was employed to enrich for the cell population identified as IDO1+ from 4T1 lung metastases for incorporation into Matrigel plugs. Plots of the sequential gating employed are shown from left to right: (P1) gating on forward scatter (X axis) and side scatter (Y axis), (P2) gating on PerCP-Cy5 intensity to select for Gr-1+ cells, (P3 and P4) gating on FITC intensity to select for CD11b^{lo} and CD11b^{hi} cells respectively. The gap between the P3 and P4 gates was included to reduce the potential for cross contamination between the two sorted populations.

FIG. 13A-13B. FACS-based identification and isolation of a highly autofluorescent subpopulation of Gr1+ CD11b^{lo} cells. (FIG. 13A) Plots of the sequential gating employed to isolate autofluorescence positive cells are shown from left to right: (P1) gating on forward scatter (X axis) and side scatter (Y axis), (P2) gating on PerCP-Cy5 intensity to select for Gr1+ cells, (P3) gating on PE-Cy7 intensity to select for CD11b^{lo} cells, and (P4,5) gating on the 488/530 nM excitation/emission channel to select the AF^{hi} (autofluorescence high, P4) and AF^{lo} (autofluorescence low, P5) cell populations. (FIG. 13B) Detection of autofluorescence on different channels. Following sequential gating of Gr1+ CD11b^{lo} cells (plots 1-3 left to right), autofluorescence profiles at 5 different excitation/emission wavelengths are shown in plots 4-8.

FIG. 14A-14B. Identification of CD11c and asialo-GM1 as markers of the Ido1-expressing cell population. (FIG. 14A) Evaluation of a panel of cell surface markers on the Gr1+ CD11b^{lo} AF^{hi} population by flow cytometry. Antibodies against F480, B220, CD3ε, CD11c, asialo-GM1, Siglec-F, CD31, IFNGR1 (CD119), MHC-II and PDL1 (CD274) were used for detection on either the 633/660 (Alexa647) or 633/780 (APC-Cy7) channels. The blue line on each graph shows the histogram representative of each antibody-associated signal relative to baseline indicated by the red line. (FIG. 14B) Concurrent evaluation of the CD11c and asialo-GM1 surface markers on the Gr1+ CD11b^{lo} AF^{hi} population. Plots of the sequential gating employed are shown from left to right: (P1) gating on forward scatter (X axis) and side scatter (Y axis), (P2) gating on PerCP-Cy5 intensity to select for Gr1+ cells, (P3) gating on PE-Cy7 intensity to select for CD11b^{lo} cells, (P4) gating on 488/530 intensity to select for AF^{hi} cells, (P5,6) gating on CD11c-PE intensity (X-axis) and asialo-GM1-APC intensity (Y-axis) to select for CD11c^{hi} asialo-GM1^{hi} (upper right) and CD11c^{lo} asialo-GM1^{lo} (lower left) cells.

FIG. 15A-15D. Comparison of neovascularizing capacity of CD11c^{hi} asialo-GM1^{hi} and CD11c^{lo} asialo-GM1^{lo} cells within the AF^{hi} population. (FIG. 15A, 15C) Matrigel plug photographs paired with confocal images stained for blood vessels (anti-CAV1; Cy3) and nuclei (DAPI). Matrigel plugs were incorporated with PBS alone or with 5×10⁴ CD11c^{hi} asialo-GM1^{hi} or CD11c^{lo} asialo-GM1^{lo} cells obtained: (FIG. 15A) from WT or Ido1^{-/-} mice and implanted into WT mice or (FIG. 15C) from Il6^{-/-} or Gcn2^{-/-} mice and implanted into WT mice. Scale=100 μm. (FIG. 15B) Photographic images of Matrigel plugs resected 9 days following subcutaneous implantation into mice. Plugs were incorporated with 3-fold serial dilutions (indicated at left) of CD11c^{hi} asialo-GM1^{hi} and CD11c^{lo} asialo-GM1^{lo} cells isolated from WT mice and implanted into WT mice. (FIG. 15D) Quantitative assessment of neovascular density corresponding to images in FIG. 15C (N≥2 plugs/condition). Straight bar: ANOVA with Dunnett's test for PBS compared with other groups. Brackets: ANOVA with Sidak's test for selected pairs

FIG. 16A-16B. CD11c^{hi} asialo-GM1^{hi} IDVCs from Ido1^{-/-}, Il6^{-/-} and Gcn2^{-/-} mice exhibit an impaired capacity to support neovascularization that is dependent upon host IFNγ. (FIG. 16A) Photographic images (left) of Matrigel plugs incorporated with CD11c^{hi} asialo-GM1^{hi} cells resected 9 days following subcutaneous implantation into mice paired with confocal images (right) of sections cut from each Matrigel plug and stained for blood vessels (anti-CAV1; Cy3) and nuclei (DAPI). From top to bottom, rows show Matrigel plugs introduced into WT and Ifng^{-/-} mice. From left to right, columns show Matrigel plugs incorporated with cells obtained from WT, Ido1^{-/-}, Il6^{-/-} and Gcn2^{-/-} mice. Scale bars=100 μm. These images are associated with the quantitative assessment of neovascular density shown in FIG. 7E. (FIG. 16B) Detection of IDO1 in CD11c^{hi} asialo-GM1^{hi} cells isolated from different transgenic mouse strains. Confocal images of isolated CD11c^{hi} asialo-GM1^{hi} cells adhered to slides and stained with either secondary Cy3-conjugated antibody (first slide) or anti-IDO1 (Cy3) and nuclei (DAPI). Scale bars=50 μm. Transgenic mouse strains (WT, Ido1^{-/-}, Gcn2^{-/-}, Il6^{-/-}, Ifng^{-/-}) from which the cells were isolated are listed at the top of each image.

FIG. 17A-17B. Flow cytometry-based validation of fluorescence signals obtained with antibodies used to identify and isolate IDVCs. (FIG. 17A) Fluorescence minus one verification of CD11c and asialo-GM1 detection on IDVCs from 4T1 lung metastases. Plots from the sequential gating strategy employed to identify and isolate CD11c^{hi} asialo-GM1^{hi} IDVCs are shown from left to right: (P1) gating on forward scatter (X axis) and side scatter (Y axis), (P2) gating on PerCP-Cy5 intensity (X axis) to select the Gr1+ population, (P3) gating on PE-Cy7 intensity (X axis) to select the CD11b^{lo} population, (P4) gating on the 488/530 nm excitation/emission channel to select the AF^{hi} population, and (P5,6) gating on PE intensity (Y axis) and Alexa647 intensity (X axis) to separate the CD11chi asialo-GM1^{hi} and CD11c^{lo} asialo-GM1^{lo} populations. (top row) all antibodies present, (middle row) no anti-CD11 c antibody included, (bottom row) no anti-asialo-GM1 antibody included. (FIG. 17B) Fluorescence minus one verification of CD45 detection on IDVCs from 4T1 lung metastases. Plots from the sequential gating strategy employed to identify and isolate CD11c^{hi} asialoGM1^{hi} IDVCs modified as follows: (P1) gating on forward scatter (X axis) and side scatter (Y axis), (P2) gating on APC-Cy7 intensity (X axis) to select the CD45+ population, (P3) gating on Pacific Blue intensity (X axis) to select the Gr-1+ population, (P4) gating on PE-Cy7 intensity (X axis) to select the CD11b^{lo} population, (P5) gating on the 488/530 nm excitation/emission channel to select the AF^{hi} population, and (P6,7) gating on PE intensity (Y axis) and Alexa647 intensity (X axis) to separate the CD11c^{hi} asialo-GM1^{hi} and CD11c^{lo} asialo-GM1^{lo} populations. (top row) all antibodies present, (bottom row) no anti-CD45 antibody included.

FIG. 18A-18C. Verification of the CD11b^{lo} status of IDVCs. (FIG. 18A) Fluorescence minus one assessment of CD11b staining of CD11c^{hi} asialo-GM1^{hi} AF^{hi} IDVCs from 4T1 lung metastases. Plots from the modified sequential gating strategy employed to identify CD11c^{hi} asialo-GM1^{hi} AF^{hi} CD11b^{lo} IDVCs are shown from left to right: (P1) gating on forward scatter (X axis) and side scatter (Y axis), (P2) gating on PE intensity (Y axis) and Alexa647 intensity (X axis) to select CD11c^{hi} asialo-GM1^{hi} cells, (P3,4) gating on the 488/530 nm excitation/emission channel (X axis) and PE-Cy7 intensity (Y axis) to separate AF^{hi} CD11c^{hi} and AF^{hi} CD11b^{lo} cells. (top row) all antibodies present, (bottom row) no anti-CD11b antibody included. The far right column shows overlays from the previous column demonstrating (top) shifts in PE-Cy7 intensity of both the P3 and P4 gated populations relative to the CD11b unstained population and (bottom) overlap of 488/530 autofluorescence between the P3 and P4 gated populations and the CD11b unstained population. (FIG. 18B) FACS sorted AF^{hi} CD11c^{hi} and AF^{hi} CD11b^{lo} cells isolated as shown in FIG. 18A, adhered to slides, and stained for IDO1 (Cy3) and DAPI for confocal imaging (Scale bars=50 μm). (FIG. 18C) Photographic images (left) of Matrigel plugs resected 9 days following subcutaneous implantation into WT mice paired with confocal images (right) of frozen sections cut from each Matrigel plug and stained for blood vessels (anti-CAV1; Cy3) and nuclei (DAPI). Rows from top to bottom show examples of Matrigel plugs incorporated with AF^{hi} CD11c^{hi} or AF^{hi} CD11b^{lo} cells.

FIG. 19A-19C. Flow cytometry-based evaluation of the Gr-1, Ly6C and Ly6G status of IDVCs compared with MDSCs. FIG. 19A is a representative example of the bifurcated gating strategy used to identify both conventional MDSCs and IDVCs from 4T1 lung metastases as follows: (P1 top) initial gating on forward scatter (X axis) and side scatter (Y axis) followed by either (P2 left) gating on Pacific Blue intensity (X axis) and PE-Cy7 intensity (Y axis) to select the CD45^{hi} CD11b^{hi} population (MDSCs) or (P2 right) gating on Pacific Blue intensity (X axis) to select the CD45+ population, (P3) gating on PE intensity (X axis) and Alexa647 intensity (Y axis) to select the CD11c^{hi} asialo-GM1^{hi} population, and (P4) gating on the 488/530 nm excitation/emission channel (X axis) and PE-Cy7 intensity (Y axis) to select the AF^{hi} CD11b^{lo} population (IDVCs). The bottom three rows show streptavidin-APC-Cy7 fluorescence staining intensity on the MDSC (FIG. 19B) and IDVC (FIG. 19C) populations from three independent samples labeled with either biotin-conjugated anti-Gr-1, anti-Ly6C or anti-Ly6G antibodies (blue histograms) in comparison with streptavidin-APC-Cy7 alone (red histograms).

FIG. 20A-20F. IDO1 protects against IFNg-mediated neovascular regression by restraining induction of Nos2. (FIG. 20A) The lung metastasis survival benefit resulting from loss of IDO1 is eliminated by concomitant loss of IFNγ. Kaplan-Meier survival curves for cohorts of WT, Ido1^{-/-}, Ifng^{-/-} and Ifng^{-/-}Ido1^{-/-} mice following orthotopic engraftment of 1×10⁴ 4T1 cells (N≥17 mice) with significance assessed by 2-group log-rank test. (FIG. 20B) The lung metastasis survival benefit resulting from loss of IDO1 is similarly eliminated by concomitant loss of NOS2. Kaplan-Meier survival curves for cohorts of WT, Ido1^{-/-}, Nos2^{-/-} and Nos2^{-/-}Ido1^{-/-} mice following orthotopic engraftment of 1×10⁴ 4T1 cells (N≥9 mice) with significance assessed by 2-group log-rank test. (FIG. 20C) IDVCs lacking IDO1 regain the capacity to elicit neovascularization in Nos2^{-/-} mice. Photographic images (left) of Matrigel plugs incorporated with CD11chiasialo-GM1^{hi} cells obtained from WT or Ido1^{-/-} mice and resected 9 days following subcutaneous implantation into WT or Nos2^{-/-} mice paired with confocal images (right) of sections cut from each Matrigel plug and stained for blood vessels (anti-CAV1; Cy3). Scale bar=100 μM. (FIG. 20D) (left) Quantitative assessment of neovascular density corresponding to images from Nos2^{-/-} mice in C (N≥2 plugs/condition) plotted as mean±SEM and evaluated for statistical significance by Student's t test. (right) Quantitative assessment of neovascular density in Matrigel plugs from WT and Ifng^{-/-} mice from FIG. 7 included for comparison. ****, P<0.0001; ns, not significant. (FIG. 20E) Loss of IDO1 results in elevated NOS2 expression within lung metastases. Confocal images from 4T1 lung metastasis stained for NOS2 (FITC, green) from WT, Ido1^{-/-}, Nos2^{-/-} and Ifng^{-/-} mice. (FIG. 20F) IDO1 inhibition similarly elicits NOS2 elevation within lung metastases. Confocal images from 4T1 lung metastasis stained for NOS2 (FITC, green) and nuclei (DAPI, blue) from WT mice dosed p.o. with either vehicle or 50 mg/kg epacadostat b.i.d. over 3 days. Dashed white lines in top panels delineate magnified regions shown in corresponding bottom panels.

### DETAILED DESCRIPTION OF THE INVENTION

The compositions and methods described herein relate to the inventors' identification of IDVCs in retinas. Induction of IDO1 in IDVCs results in Integrated Stress Response (ISR) activation which is central to IDVCs' ability to support the establishment and maintenance of neovascularization.

In addition to immunosuppression, it is generally accepted that MDSCs (myeloid-derived suppressor cells) also support tumor angiogenesis. Recently, the tryptophan catabolizing enzyme IDO1 (indoleamine 2,3-dioxygenase) has been implicated in promoting neovascularization through its positioning as a key regulatory node between the inflammatory cytokines IFNγ (interferon-y) and IL6 (interleukin 6). However, the cell type in which IDO1 is expressed has not been reported previously. It is reported herein that within the heterogeneous expanse of Gr-1+ MDSCs, the ability to elicit neovascularization in vivo is predominantly associated with a minor subset of highly autofluorescent CD11b^{lo} cells. IDO1 expression is further restricted to a discrete CD11c, asialo-GM1 double positive subpopulation of these cells, designated here as IDVCs (IDO1-dependent vascularizing cells) due to the dominant role that the IDO1 activity in these cells plays in promoting neovascularization. Mechanistically, the induction of IDO1 in IDVCs provides a negative feedback constraint on the anti-angiogenic effect of host IFNγ by signaling for the production of IL6 through GCN2-mediated activation of the integrated stress response within these cells. These findings reveal fundamental molecular and cellular insights into how IDO1 interfaces with the inflammatory milieu to promote neovascularization.

### I. Components of the Compositions and Methods

In the descriptions of the compositions and methods discussed herein, the various components can be defined by use of technical and scientific terms having the same meaning as commonly understood by one of ordinary skill in the art to which this invention belongs and by reference to published texts. Such texts provide one skilled in the art with a general guide to many of the terms used in the present application. The definitions contained in this specification are provided for clarity in describing the components and compositions herein and are not intended to limit the claimed invention.

### A. Subject

“Patient” or “subject” or “individual” as used herein means a mammalian animal, including a human, a veterinary or farm animal, a domestic animal or pet, and animals normally used for clinical research. In one embodiment, the subject of these methods and compositions is a human. In one embodiment, the subject has an ocular disease. In another embodiment, the subject has an ocular disease and has yet to be treated with any therapy. In another embodiment, the subject has an ocular disease and is treated with conventional methodologies, e.g., administration of vascular endothelial growth factor (VEGF) inhibitors intraocularly, but is not responding to the treatment optimally or in a manner sufficient to achieve a sufficient therapeutic benefit. In another embodiment, the subject having said ocular disease is receiving administration of VEGF inhibitors or blockers but is not achieving the desired therapeutically maximal response that been observed in other patients upon the administration of a VEGF blocker or inhibitor monotherapy.

### B. Ocular Disease

By the term “ocular disease” is meant a disorder or disease of the eye. In one embodiment, an ocular disease is characterized by neovascularization, i.e., new or abnormal blood vessel formation in a tissue or part of the eye, or excessive blood vessel formation is a tissue or part of the eye. In one embodiment, the ocular disorder is a retinopathy. In a particular embodiment, the ocular disease is characterized by abnormal/aberrant vascularization. In a particular embodiment, the ocular disease is characterized by intraocular neovascularization. The intraocular neovascularization may be, without limitation, neovascularization of the optic disc, iris, retina, choroid, cornea, and/or vitreous humour. Examples of ocular diseases include, without limitation, glaucoma, pannus, pterygium, macular edema, macular degeneration (e.g., age-related macular degeneration), retinopathy (e.g., diabetic retinopathy, vascular retinopathy, retinopathy of prematurity), diabetic retinal ischemia, diabetic macular edema, retinal degeneration, retrolental fibroplasias, corneal graft neovascularization, central retinal vein occlusion, pathological myopia, uveitis, inflammatory diseases of the eye, and proliferative vitreoretinopathy. In a particular embodiment, the ocular disease is selected from the group consisting of retinopathy (e.g., retinopathy of prematurity, diabetic retinopathy (e.g., proliferative diabetic retinopathy) and macular degeneration (e.g., dry or wet macular degeneration). In yet another embodiment, the ocular disease or disorder is macular dystrophy (e.g., Stargardt's, Vitelliform macular dystrophy (VTM), North Carolina macular dystrophy, or Best disease).

Age-related macular degeneration (AMD) is a degenerative disease of the macula, often leading to progressive vision loss. It is diagnosed as either dry (non-neovascular) or wet (neovascular). The rate of disease progression can vary among individuals and has been associated with multiple risk factors. Early-stage AMD includes clinical signs such as drusen, and abnormalities of the retinal pigment epithelium. Late-stage AMD can be neovascular (also known as wet or exudative) or non-neovascular (known as atrophic, dry, or non-exudative). Late non-neovascular AMD is characterized by the development of geographic atrophy (GA) of the macula and loss of central visual acuity, leading to severe and permanent visual impairment and legal blindness (See, e.g., Mitchell, P. et al. (2018). Age-related macular degeneration. The Lancet, 392(10153), 1147-1159, which is incorporated herein by reference). Late neovascular AMD is characterized by the development of edema and retinal detachment with loss of central visual acuity, leading to severe and permanent visual impairment and legal blindness.

### C. IDO-Dependent Vascularizing Cells (IDVCs)

Described herein is a newly isolated cell type, termed IDO1-dependent vascularizing cells (IDVCs). This novel immune cell subtype was isolated in mice from within the heterogeneous Gr-1+ MDSC population and is functionally distinguishable from the bulk of MDSCs by its ability to elicit neovascularization and sustain these new blood vessels in the presence of IFNγ through ISR-driven production of IL6 activated by IDO1.

In addition to immunosuppression, it is generally accepted that MDSCs (myeloid-derived suppressor cells) also support tumor angiogenesis. Recently, the tryptophan catabolizing enzyme IDO1 (indoleamine 2,3-dioxygenase) has been implicated in promoting neovascularization through its positioning as a key regulatory node between the inflammatory cytokines IFNγ (interferon-γ) and IL6 (interleukin 6). As described herein, within the heterogeneous expanse of Gr-1⁺ MDSCs, the ability to elicit neovascularization in vivo is predominantly associated with a minor subset of highly autofluorescent CD11b^{lo} cells. IDO1 expression is further restricted to a discrete CD11c, asialo-GM1 double positive subpopulation of these cells, designated here as IDVCs due to the dominant role that the IDO1 activity in these cells plays in promoting neovascularization. Mechanistically, the induction of IDO1 in IDVCs provides a negative feedback constraint on the anti-angiogenic effect of host IFNγ by signaling for the production of IL6 through GCN2-mediated activation of the integrated stress response within these cells. In one embodiment, the IDVCs are located in the retina of the eye. In another embodiment, the IDVCs are located in the choroid of the eye. In another embodiment, the IDVCs are located in the retina and the choroid of the eye.

Induction of IDO1 in IDVC cells results in ISR (Integrated Stress Response (ISR)) activation which is central to IDVCs' ability to support the establishment and maintenance of neovascularization. IDVCs can be isolated to near homogeneity from mice by flow cytometry using antibodies to detect high levels of CD45, CD11c, asialo-GM1 and Gr-1 and a low level of CD11b on the cell surface coupled with their relatively high level of endogenous autofluorescence.

IDVCs are leukocytes or another cell type which respond to cytokines, including the cytokine IFNγ (interferon-gamma), by inducing IDO1 and which are functionally characterized as having a role in neovascularization, the establishment and maintenance of which requires the induction of IDO1 within the IDVCs. Characterization of the cells as IDVC may include isolating the cells and incorporating them into Matrigel plugs that are implanted under the skin of mice. After several days, the sites of implantation are evaluated for the presence of blood vessels.

In one embodiment, the IDVCs are characterized by the following phenotype: CD11b+CD14−CD15+ or CD11b+CD14−CD66b+. Alternatively, the CD33 myeloid marker can be used instead of CD11b since very few CD15+ cells are CD11b−. In another embodiment, the IDVCs are characterized by the following phenotype: Lin−HLA-DR−/loCD33+ or Lin−HLA-DR−/loCD11b+CD14−CD15+CD33+. In one embodiment, one or more of the following cell surface markers are located on the IDVCs: CD11c, CD274 (PDL1), MHC class II, CD4, CD31 (PECAM-1), CD202B (TIE2), CD205 (DEC-205), Siglec 8, or EMR1.

### D. Integrated Stress Response (ISR) Pathway Nodes

The integrated stress response (ISR) is a ubiquitous signaling pathway inducible in eukaryotic cells, which is activated in response to a range of physiological stimuli and pathological conditions. Such stimuli commonly include cell extrinsic factors and stressors such as hypoxia, amino acid deprivation, glucose deprivation, and viral infection. In addition, cell intrinsic stresses such as endoplasmic reticulum (ER) stress, caused by the accumulation of unfolded proteins in the ER, can also activate the ISR. Furthermore, in the context of cancer biology, the ISR can be triggered by oncogene activation.

As described herein, it is shown that the ISR is the biologically relevant signaling pathway through which IDO1 acts to promote pathological neovascularization in retinopathies. Inhibition of key ISR nodes, such as GCN2, CHOP, and ATF4, was shown to reduce neovascularization in an animal model of retinopathies, namely the Oxygen Induced Retinopathy (OIR) mouse model. As used herein, the term “ISR node” refers to any component along the signaling pathway initiated by stress-activated eIF2α kinases. In particular embodiments, ISR node refers to GCN2, CHOP, and/or ATF4.

General control nonderepressible 2 (GCN2) is a master regulator kinase of amino acid homeostasis and important for cancer survival in the tumor microenvironment under amino acid depletion. GCN2 is also designated in humans as eukaryotic translation initiation factor 2-alpha kinase 4 (EIF2AK4), eIF-2-alpha kinase GCN2, and GCN2-like protein, and is encoded by the EIF2AK4 gene. GCN2 is a metabolic-stress sensing protein kinase that phosphorylates the alpha subunit of eukaryotic translation initiation factor 2 (EIF2S1/eIF-2-alpha) in response to low amino acid availability. The sequence of hGCN2 is known in the art and can be found, e.g., UniProtKB—Q9P2K8.

C/EBP homologous protein (CHOP), also known as growth arrest and DNA damage-inducible protein 153 (GADD153), belongs to the CCAAT/enhancer-binding protein (C/EBP) family. CHOP dimerizes with other C/EBP members and changes their DNA-binding and transactivation properties. It induces growth arrest and apoptosis after endoplasmic reticulum stress or DNA damage. The sequence of hCHOP is known in the art and can be found, e.g., UniProtKB−P35638.

Activating Transcription Factor 4 (ATF4) is a transcription factor that binds the cAMP response element (CRE) (consensus: 5′-GTGACGT[AC][AG]-3′) and acts both as a regulator of normal metabolic and redox processes, and as a master transcription factor during the integrated stress response (ISR) (PubMed:1847461, PubMed:16682973, PubMed:31444471, PubMed:32132707). ATF4 is a core effector of the ISR, which is required for adaptation to various stress, such as endoplasmic reticulum (ER) stress, amino acid starvation, mitochondrial stress or oxidative stress. The sequence of hATF4 is known in the art and can be found, e.g., UniProtKB—P18848.

### E. IDO1

Indoleamine 2, 3-dioxygenase (IDO1) is a tryptophan catabolic enzyme that catalyzes the first step of the conversion of tryptophan into kynurenine. IDO1 is an extrahepatic enzyme that catabolizes the essential amino acid tryptophan independently of metabolic processing of tryptophan in the liver. IDO1 monotherapy for treatment of ocular disorders is described in the publication WO2016/100851; see also, U.S. Pat. No. 10,535,035, incorporated by reference herein. IDO1 is sometimes referred to herein as IDO.

### F. VEGF and VEGFR

“VEGF” refers to a vascular endothelial growth factor that induces angiogenesis or an angiogenic process. As used herein, the term “VEGF” includes the various subtypes or isoforms of VEGF (also known as vascular permeability factor (VPF) and VEGF-A) that arise by, e.g., alternative splicing of the VEGF-A/VPF gene including VEGF121, VEGF165 and VEGF189. Further, as used herein, the term “VEGF” includes VEGF-B, VEGF-C, VEGF-D and VEGF-E, which act through a cognate VEFG receptor (i.e., VEGFR) to induce angiogenesis or an angiogenic process. The term “VEGF” includes any member of the class of growth factors that binds to a VEGF receptor such as VEGFR-1 (Flt-1), VEGFR-2 (KDR/Flk-1), or VEGFR-3 (FLT-4). The term “VEGF” can be used to refer to a “VEGF” polypeptide or a “VEGF” encoding gene or nucleic acid. See, US Patent Publication 2019/0380187, incorporated by reference herein.

### G. Inhibitor, Blocker, Antagonist

By the general terms “blocker”, “inhibitor”, or “antagonist” is meant an agent that inhibits, either partially or fully, the activity or production of a target molecule or target cell, e.g., as used herein, IDVCs, an ISR node, IDO1, or VEGF. In particular embodiments, these terms refer to a composition or compound or agent capable of decreasing levels of gene expression, mRNA levels, protein levels or protein activity of the target molecule. Illustrative forms of antagonists include, for example, proteins, polypeptides, peptides (such as cyclic peptides), antibodies or antibody fragments, peptide mimetics, nucleic acid molecules, antisense molecules, ribozymes, aptamers, RNAi molecules, and small organic molecules. Illustrative non-limiting mechanisms of antagonist inhibition include repression of ligand synthesis and/or stability (e.g., using, antisense, ribozymes or RNAi compositions targeting the ligand gene/nucleic acid), blocking of binding of the ligand to its cognate receptor (e.g., using anti-ligand aptamers, antibodies or a soluble, decoy cognate receptor), repression of receptor synthesis and/or stability (e.g., using, antisense, ribozymes or RNAi compositions targeting the ligand receptor gene/nucleic acid), blocking of the binding of the receptor to its cognate receptor (e.g., using receptor antibodies), blocking of the activation of the receptor by its cognate ligand (e.g., using receptor tyrosine kinase inhibitors), the blocking of an active site of an enzyme as a result of the enzyme binding a molecule that prevents, i.e., inhibits, the binding of the natural substrate, or the interaction of an inhibitor molecule that binds either to the active or binding site of a protein, or through allosteric sites on a protein, to modulate the biological activity of the protein. In addition, the blocker or inhibitor may directly or indirectly inhibit the target molecule.

### i. Salts

The compositions described herein also includes all salts of the specific IDVC, ISR node, IDO, or VEGF inhibitor compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid) salts of basic residues such as amines; alkali (such as Li, Na, K, Mg, Ca) or organic (such as trialkyl ammonium) salts of acidic residues such as carboxylic acids; and the like. The salts of compounds described or referenced herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The “pharmaceutically acceptable salts” of compounds described herein or incorporated by reference include a subset of the “salts” described above which are, conventional non-toxic salts of the parent compound formed, for example, from non-toxic inorganic or organic acids. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### ii. Prodrug

By the term “prodrug” is meant a compound or molecule or agent that, after administration, is metabolized (i.e., converted within the body) into the parent pharmacologically active molecule or compound, e.g., an active IDO inhibitor (see, e.g., International Publication WO2019/051198), a VEGF or VEGFR inhibitor or antagonist, ISR node antagonist or inhibitor. Prodrugs are substantially, if not completely, in a pharmacologically inactive form that is converted or metabolized to an active form (i.e., drug) - such as within the body or cells, typically by the action of, for example, endogenous enzymes or other chemicals and/or conditions. Instead of administering an active molecule directly, a corresponding prodrug is used to improve how the composition/active molecule is absorbed, distributed, metabolized, and/or excreted. Prodrugs are often designed to improve bioavailability or how selectively the drug interacts with cells or processes that are not its intended target. This reduces adverse or unintended undesirable or severe side effects of the active molecule or drug.

### iii. Biosimilar

A “biosimilar” is a biological product, generally a large and complex molecule, which may be produced from living organisms, and monitored to ensure consistent quality that is highly similar to a reference product, e.g., an already FDA-approved biological drug. A biosimilar that receives FDA approval must have no clinically meaningful differences from the reference drug in purity, safety, molecular structure and bioactivity, or potency.

### iv. Antibody and Fragments

By the term “antibody” or “antibody molecule” is any peptide or protein, including antibodies and fragments thereof, that binds to a specific antigen. As used herein, antibody or antibody molecule contemplates intact immunoglobulin molecules, immunologically active portions of an immunoglobulin molecule, and fusions of immunologically active portions of an immunoglobulin molecule.

The antibody may be a naturally occurring antibody or may be a synthetic or modified antibody (e.g., a recombinantly generated antibody; a chimeric antibody; a bispecific antibody; a humanized antibody; a camelid antibody; and the like). The antibody may comprise at least one purification tag. In a particular embodiment, the framework antibody is an antibody fragment. The term “antibody fragment” includes a portion of an antibody that is an antigen binding fragment or single chains thereof An antibody fragment can be a synthetically or genetically engineered polypeptide. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv). Such single chain antibodies are also intended to be encompassed within the term “antigen-binding fragment” of an antibody. These antibody fragments are obtained using conventional techniques known to those in the art, and the fragments can be screened for utility in the same manner as whole antibodies. Antibody fragments include, without limitation, immunoglobulin fragments including, without limitation: single domain (Dab; e.g., single variable light or heavy chain domain), Fab, Fab′, F(ab′)2, and F(v); and fusions (e.g., via a linker) of these immunoglobulin fragments including, without limitation: scFv, scFv2, scFv-Fc, minibody, diabody, triabody, and tetrabody. The antibody may also be a protein (e.g., a fusion protein) comprising at least one antibody or antibody fragment.

The antibodies useful in the methods are preferably “immunologically specific”, which refers to proteins/polypeptides, particularly antibodies, that bind to one or more epitopes of a protein or compound of interest, but which do not substantially recognize and bind other molecules in a sample containing a mixed population of antigenic biological molecules.

The antibodies of the instant invention may be further modified. For example, the antibodies may be humanized. In a particular embodiment, the antibodies (or a portion thereof) are inserted into the backbone of an antibody or antibody fragment construct. For example, the variable light domain and/or variable heavy domain of the antibodies of the instant invention may be inserted into another antibody construct. Methods for recombinantly producing antibodies are well-known in the art. Indeed, commercial vectors for certain antibody and antibody fragment constructs are available.

The antibodies of the instant invention may also be conjugated/linked to other components. For example, the antibodies may be operably linked (e.g., covalently linked, optionally, through a linker) to at least one cell penetrating peptide, detectable agent, imaging agent, or contrast agent. The antibodies of the instant invention may also comprise at least one purification tag (e.g., a His-tag). In a particular embodiment, the antibody is conjugated to a cell penetrating peptide.

### v. Aptamer

The term “aptamer” refers to a peptide or nucleic acid that has an inhibitory effect on a target. Inhibition of the target by the aptamer can occur by binding of the target, by catalytically altering the target, by reacting with the target in a way which modifies the target or the functional activity of the target, by ionically or covalently attaching to the target as in a suicide inhibitor or by facilitating the reaction between the target and another molecule. Aptamers can be peptides, ribonucleotides, deoxyribonucleotides, other nucleic acids or a mixture of the different types of nucleic acids. Aptamers can comprise one or more modified amino acid, bases, sugars, polyethylene glycol spacers or phosphate backbone units as described in further detail herein.

### vi. RNA and DNA

The terms “RNA interference,” “RNAi,” “miRNA,” “shRNA,”and “siRNA” refer to any method by which expression of a gene or gene product is decreased by introducing into a target cell one or more double-stranded RNAs, which are homologous to a gene of interest (particularly to the messenger RNA of the gene of interest). Gene therapy, i.e., the manipulation of RNA or DNA using recombinant technology and/or treating disease by introducing modified RNA or modified DNA into cells via a number of widely known and experimental vectors, recombinant viruses and CRISPR technologies, may also be employed in delivering, via modified RNA or modified DNA, effective inhibition of the IDO/TDO pathways and gene products and VEGF pathways and gene products to accomplish the outcomes described herein with the combination therapies described. Such genetic manipulation can also employ gene editing techniques such as CRISPR (Clustered Regularly Interspaced Short Palindromic Repeats) and TALEN (transcription activator-like effector genome modification), among others. See, for example, the textbook National Academies of Sciences, Engineering, and Medicine. 2017. Human Genome Editing: Science, Ethics, and Governance. Washington, DC: The National Academies Press. https://doi.org/10.17226/24623, incorporated by reference herein for details of such methods.

### vii. Small Molecule

The term “small molecule” when applied to a pharmaceutical generally refers to a non-biologic, organic compound that affects a biologic process which has a relatively low molecular weight, below approximately 900 daltons. Small molecule drugs have an easily identifiable structure, that can be replicated synthetically with high confidence. In one embodiment a small molecule has a molecular weight below 550 daltons to increase the probability that the molecule is compatible with the human digestive system's intracellular absorption ability. Small molecule drugs are often administered orally, as tablets. The term small molecule drug is used to contrast them with biologic drugs, which are often relatively large molecules, such as peptides, proteins and recombinant protein fusions, and which are frequently produced using a living organism.

### viii. Specific Inhibitors of IDO1

The phrase “a composition that blocks or inhibits the expression, induction, activity, and/or signaling of IDO1” includes without limitation, a small molecule enzyme inhibitor that targets IDO1 directly, or a salt, enantiomer or prodrug thereof Another embodiment of such a composition includes comprises a small molecule that blocks or inhibits one or more targets upstream or downstream of IDO1 (including ISR nodes such as GCN2, CHOP10, and/or ATF4) in their respective pathways to inhibit the action of IDO1 and other tryptophan catabolizing enzymes, or a salt, enantiomer or prodrug thereof In another embodiment, the IDO1 inhibitor compositions comprise a molecule that mimics the presence of tryptophan. In still another embodiment, the composition containing the IDO inhibitors comprise a nucleic acid molecule that inhibits the translation or transcription of IDO1, for example an siRNA or shRNA. In another embodiment, the IDO1 inhibitor compositions comprise a protein therapeutic that binds to and inhibits the activity of IDO1. Such a protein therapeutic can include an anti-IDO1 antibody, or binding fragment thereof.

More specifically, a composition that blocks or inhibits the expression, induction, activity, and/or signaling of IDO1 includes, without limitation, at least one of the following compounds or a pro-drug, salt, and/or any therapeutically effective formulation of:
Indoximod (1-methyl-D-tryptophan, 1MT, NLG-8189)
1-methyl-L-tryptophan
a racemic mixture of 1-methyl-D-tryptophan and 1-methyl-L-tryptophan
Epacadostat (INCB024360; Incyte; Wilmington, DE; described in Liu et al. (2010) Blood 115(17):3520-3530; Koblish et al. (2010) Mol. Cancer Ther., 9(2):489-498))
Navoximod (NLG-919, GDC-0919, RG6078; Lumos Pharma/NewLink Genetics/Genentech),
Indoximod prodrug NLG802
BMS-986205/ONO-7701 (F001287, Hunt et al., AACR 2017, Abstract 4964),
PF-06840003/EOS200271 (EOS, Wythes et al, SITC 2016, Abstract 253),
A compound of the formula, 1-R-D-tryptophan or 1-R-L-tryptophan, wherein R is a C1-C12 alkyl;
MK-7162/IOM2983 (Merck and Co.)
LY3381916 (Lilly)
KHK2455 (Kyowa Hakko Kirin)
HTI-1090/SHR9146 (Hengrui Therapeutics, Inc)
DN1406131 (De Novo Pharmatech)
RG70099 (Roche/CuraDev)
Roxyl-WL (Xu et al. J Enzyme Inhib Med Chem. 2018; 33(1): 1089-94.)
TPST-8844 (Tempest Therapeutics)
Ethyl pyruvate
AMG-1 (Amgen), as described in Smith J R et al, Novel indoleamine 2,3-dioxygenase-1 inhibitors from a multistep in silico screen, Bioorganic & Medicinal Chemistry, 20(3): 1354-1363 1 February 2012, incorporated by reference herein;
methylthiohydantoin-DL-tryptophan (MTH-Trp),
β-(3-β)-DL-alanine,
β-(3-benzo(b)thienyl)-DL-alanine,
6-nitro-L-tryptophan,
indole 3-carbinol,
3,3′-diindolylmethane,
epigallocatechin gallate,
5-Br-4-Cl-indoxyl 1,3-diacetate,
9-vinylcarbazole, acemetacin,
5-bromo-DL-tryptophan,
5-bromoindoxyl diacetate,
Naphthoquinone-based,
S-allyl-brassinin,
S-benzyl-brassinin,
5-Bromo-brassinin,
Phenylimidazole-based,
4-phenylimidazole,
Exiguamine A
NSC401366
beta-lapachone (3,4-dihydro-2,2-dimethyl-2H-naphthol[1,2-b]pyran-5,6-dione, Flick et al. (2013) Int. J. Tryp. Res. 6:35-45) (ARQ 761; ArQule, now owned by Merck);
DX-03-12 and other compounds described in Wen, H. et al, Design and Synthesis of Indoleamine 2,3-Dioxygenase 1 Inhibitors and Evaluation of Their Use as Anti-Tumor Agents, Molecules 2019, 24, 2124; doi:10.3390/molecules -24112124, incorporated by reference herein;
Compounds described in patent WO2014/186035 and US Patent Publication No. US 2018/030026 (Curadev); and/or
Compounds described in WO2014/081689 and U.S. Pat. No. 9,499,497 (Vertex Pharmaceuticals).

Still other suitable inhibitors are identified in Wang, X-X et al, Recent advances in the discovery of indoleamine 2,3-dioxygenase 1 (IDO1) inhibitors, MedChemComm, and grouped as tryptophan derivatives, inhibitors with an imidazole, 1,2,3-triazole or tetrazole scaffold, inhibitors with quinone or iminoquinone, N-hydroxyamidines.

Still other examples of small molecule IDO1 inhibitors are provided, without limitation, in PCT/US2014/022680 (e.g., tricyclic compounds related to imidazoisoindoles; compounds of Formulas I-V), PCT/US2012/033245 (e.g., fused imidazole derivatives; compounds of Formula I or II), PCT/US2010/054289 (e.g., imidazole derivatives; compounds of Formulas I-VIII), PCT/US2009/041609 (e.g., compounds of Formulas I-VIII), PCT/US2008/57032 (e.g., napthoquinone derivatives; compounds of Formula I, II, or III), PCT/US2008/085167 (e.g., compounds of Formulas I-XLIV), PCT/US2006/42137 (e.g., compounds of Formula I), PCT/US2006/017983 (e.g., compounds of Formula I), PCT/US2004/005155 (e.g., phenyl-TH-DL-trp (3-(N-phenyl-thiohydantoin)-indole), propenyl-TH-DL-trp (3-(N-allyl-thiohydantoin)-indole), and methyl-TH-DL-trp (3-(N-methyl-thiohydantoin)-indole)), PCT/US2004/005154 (e.g., compounds of Formula I or II), U.S. Pat. No. 7,705,022 (e.g., compounds of Formula I), U.S. Pat. No. 8,008,281 (e.g., phenyl-TH-DL-trp (3-(N-phenyl-thiohydantoin)-indole), propenyl-TH-DL-trp (3-(N-allyl-thiohydantoin)-indole), and methyl-TH-DL-trp (3-(N-methyl-thiohydantoin)-indole)), U.S. Pat. No. 7,714,139 (e.g., compounds of Formula I or II), U.S. Patent Application Publication No. 20140066625 (e.g., fused imidazole derivatives; compounds of Formula I or II), U.S. Patent Application Publication No. 20130177590 (e.g., N-hydroxyamidinoheterocycles; compounds of Formulas I-III), U.S. Patent Application Publication No. 20140023663 (e.g., 1,2,5-oxadiazoles; compounds of Formula I), U.S. Patent Application Publication No. 20080146624 (e.g., amidines; compounds of Formulas I or II), U.S. Patent Application Publication No. 20080119491 (e.g., amidinoheterocycles; compounds of Formulas I-IV), U.S. Patent Application Publication No. 20080182882 (e.g., N-hydroxyamidinoheterocycles; compounds of Formula I), U.S. Patent Application Publication No. 20080214546 (e.g., N-hydroxyamidinoheterocycles; compounds of Formula I), U.S. Patent Application Publication No. 20060258719 (compounds of Formula I), Banerjee et al. (2008) Oncogene 27:2851-2857 (e.g., brassinin derivatives;), and Kumar et al. (2008) J. Med. Chem., 51:1706-1718 (e.g., phenyl-imidazole-derivatives). In a particular embodiment, the IDO1 inhibitor is a prodrug (see, e.g., U.S. Patent Application Publication No. 20170022157 and U.S. Provisional Application No. 62/555,726). All references are incorporated by reference herein, particularly for the IDO1 inhibitors provided therein.

In a particular embodiment, the IDO1 induction inhibitor is ethyl pyruvate (Muller, et al. (2010) Cancer Res. 70:1845-1853) or gleevec (imatinib, Balachandran et al. (2011) Nat. Med. 17:1094-1100). In a particular embodiment, the IDO1 pathway inhibitor (e.g., inhibitor of downstream signaling pathway) is 1-methyl-tryptophan, particularly 1-methyl-D-tryptophan (indoximod, NLG-8189; 1-methyl-D-tryptophan; NewLink Genetics), including salts and prodrugs (U.S. Patent Application Publication No. 20170022157) or a racemic mix comprising the same.

Further inhibitors of IDO1 expression include, without limitation, inhibitors of JAK/STAT (e.g., JAK, STAT3, STAT1) (Du et al. (2000) J. Interferon Cytokine Res., 20:133-142, Muller et al. (2005) Nature Med., 11:312-319; Yu et al. (2014) J. Immunol., 193:2574-2586) , NFKB (Muller et al. (2005) Nature Med., 11:312-319; Muller et al. (2010) Cancer Res., 70:1845-1853), KIT (Balachandran et al. (2011) Nature Med., 17:1094-1100), MET (Rutella et al. (2006) Blood 108:218-227; Giannoni et al. (2014) Haematologica 99:1078-1087), RAS/RAF/MEK (Liu (2010) Blood 115:3520-3530), aryl hydrocarbon receptor (AHR) (Bessede et al. (2014) Nature 511:184-190; Litzenburger et al. (2014) Oncotarget 5:1038-1051), or vascular endothelial growth factor receptor (VEGFR) (Marti et al. (2014) Mem Inst Oswaldo Cruz 109:70-79). In a particular embodiment, the inhibitor is not an inhibitor of VEGFR.

Still other IDO inhibitors, salts or prodrugs thereof are described in the following US Patents, incorporated by reference herein:
U.S. Pat. No. 10,501,458 Substituted bicyclic fused ring compounds as indoleamine-2,3-dioxygenase inhibitors;
U.S. Pat. No. 10,494,360 Inhibitors of indoleamine 2,3-dioxygenase;
U.S. Pat. No. 10,472,336 Modulators of indoleamine 2,3-dioxygenase;
U.S. Pat. No. 10,436,785 Anti-indoleamine 2,3-dioxygenase 1 antibodies and diagnostic uses thereof;
U.S. Pat. No. 10,399,933 Inhibitors of indoleamine-2,3-dioxygenase for the treatment of cancer;
U.S. Pat. No. 10,399,932 Inhibitors of indoleamine-2,3-dioxygenase for the treatment of cancer
U.S. Pat. No. 10,369,137 1,2,5-Oxadiazoles as inhibitors of indoleamine 2,3-dioxygenase
U.S. Pat. No. 10,358,427 Modulators of indoleamine 2,3-dioxygenase
U.S. Pat. No. 10,336,731 Substituted 1H-indole-2-carboxamide compounds as indoleamine-2,3-dioxygenase inhibitors
U.S. Pat. No. 10,329,297 Compounds for the inhibition of indoleamine-2,3-dioxygenase
U.S. Pat. No. 10,323,004 Inhibitors of indoleamine 2,3-dioxygenase and methods of their use
U.S. Pat. No. 10,287,252 Inhibitors of tryptophan-2,3-dioxygenase or indoleamine-2,3-dioxygenase
U.S. Pat. No. 10,280,163 5 or 8-substituted imidazo[1,5-a] pyridines as indoleamine and/or tryptophane 2,3-dioxygenases
U.S. Pat. No. 10,280,157 Process for the synthesis of an indoleamine 2,3-dioxygenase inhibitor
U.S. Pat. No. 10,239,894 Inhibitors of indoleamine 2,3-dioxygenase
U.S. Pat. No. 10,208,002 Modulators of indoleamine 2,3-dioxygenase and methods of using the same
U.S. Pat. No. 10,034,864 1,2,5-oxadiazoles as inhibitors of indoleamine 2,3-dioxygenase
U.S. Pat. No. 9,873,688 Process for the synthesis of an indoleamine 2,3-dioxygenase inhibitor
U.S. Pat. No. 9,789,094 1,2,5-oxadiazoles as inhibitors of indoleamine 2,3-dioxygenase
U.S. Pat. No. 9,771,370 Compounds for the inhibition of indoleamine-2,3-dioxygenase
U.S. Pat. No. 9,675,571 Inhibitors of indoleamine 2,3-dioxygenase (IDO)
U.S. Pat. No. 9,499,497 Compounds useful as inhibitors of indoleamine 2,3-dioxygenase
U.S. Pat. No. 9,463,239 Use of inhibitors of indoleamine-2,3-dioxygenase in combination with other therapeutic modalities
U.S. Pat. No. 9,433,666 Indoleamine 2,3-dioxygenase based immunotherapy
U.S. Pat. No. 9,321,755 Process for the synthesis of an indoleamine 2,3-dioxygenase inhibitor
U.S. Pat. No. 9,320,732 1,2,5-oxadiazoles as inhibitors of indoleamine 2,3-dioxygenase
U.S. Pat. No. 9,073,875 Compounds useful as inhibitors of indoleamine 2,3-dioxygenase
U.S. Pat. No. 8,993,605 1,2,5-oxadiazoles as inhibitors of indoleamine 2,3-dioxygenase
U.S. Pat. No. 8,951,536 N-hydroxyamidinoheterocycles as modulators of indoleamine 2,3-dioxygenase
U.S. Pat. No. 8,846,726 Modulators of indoleamine 2,3-dioxygenase and methods of using the same
U.S. Pat. Nos. 8,822,511 and 8,796,319 1,2,5-oxadiazoles as inhibitors of indoleamine 2,3-dioxygenase
U.S. Pat. No. 8,580,844 Use of inhibitors of indoleamine-2,3-dioxygenase in combination with other therapeutic modalities
U.S. Pat. Nos. 8,507,541; 8,450,351 and 8,377,976 N-hydroxyamidinoheterocycles as modulators of indoleamine 2,3-dioxygenase
U.S. Pat. No. 8,436,151 Indoleamine 2,3-dioxygenase-2 antibodies
U.S. Pat. No. 8,372,870 Modulators of indoleamine 2,3-dioxygenase and methods of using the same for treating cancer
U.S. Pat. No. 8,088,803 1,2,5-oxadiazoles as inhibitors of indoleamine 2,3-dioxygenase
U.S. Pat. No. 8,058,416 Nucleic acid molecules encoding indoleamine 2,3-dioxygenase-2
U.S. Pat. No. 8,034,953 Modulators of indoleamine 2,3-dioxygenase and methods of using the same
U.S. Pat. No. 7,799,776 Indoleamine 2,3-dioxygenase (IDO) inhibitors
U.S. Pat. No. 7,598,287 Use of inhibitors of indoleamine-2,3-dioxygenase in combination with other therapeutic modalities.

In a yet another embodiment, the IDO inhibitor is an IDO-targeting, peptide-based vaccine (such as described in Iversen et al. (2014) Clin. Cancer Res., 20:221-32).

### ix. Specific Inhibitors of ISR Nodes

GCN2 inhibitors include, without limitation, GCN2-IN-1 (A-92) (Medchemexpress), GCN2iA (Nakamura et al, Inhibition of GCN2 sensitizes ASNS-low cancer cells to asparaginase by disrupting the amino acid response, PNAS, epub Jul. 30, 2018 115 (33) E7776-E7785), those described by Fujimoto et al (Identification of Novel, Potent, and Orally Available GCN2 Inhibitors with Type I Half Binding Mode, ACS Med Chem Lett. 2019 Oct 10; 10(10): 1498-1503, epub Sept 2019), GZD824 (Kato et al, GZD824 inhibits GCN2 and sensitizes cancer cells to amino acid starvation stress, Molecular Pharmacology, 98(6) (Oct. 8, 2020)), inhibitors based on a triazolo[4,5-d]pyrimidine scaffold such as those described by Lough et al (Triazolo[4,5-d]pyrimidines as Validated General Control Nonderepressible 2 (GCN2) Protein Kinase Inhibitors Reduce Growth of Leukemia Cells, Volume 16, September 2018, Pages 350-360), GCN2 inhibitors being tested by RAPT therapeutics (e.g., GCN2i-282, GCN2i-490, FLX-GCN2i-A, FLX-GCN2i-B) (Marshall et al, Targeting the Stress Response Kinase GCN2 to Restore Immunity And Decrease Tumor Cell Survival SITC 2019). Other GCN2 inhibitors include those disclosed in US Patent Publication No. 20190375753, including those having the general formula (Rapt Therapeutics):

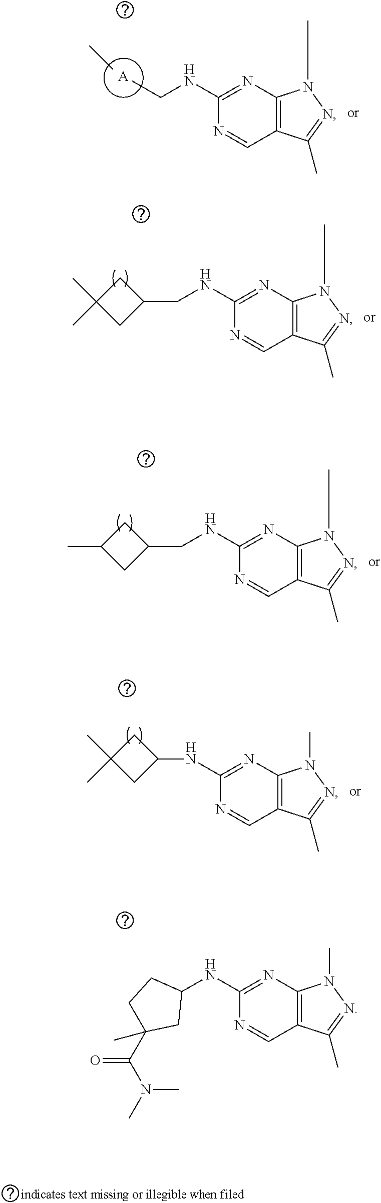

Each of these documents is incorporated herein by reference.

CHOP inhibitors include, without limitation, oligonucleotides such as those described by Klar et al (Abstract 3275: Inhibition of ER-stress factor C/EBP homologous protein (Chop) with LNAplus™ antisense-oligonucleotides to improve immunotherapy of cancer, DOI: 10.1158/1538-7445.AM2019-3275 Published July 2019), and ISRIB.

ATF4 inhibitors include, without limitation, ursolic acid, tomatidine, and derivatives thereof, Ebert et al, (Identification and Small Molecule Inhibition of an Activating Transcription Factor 4 (ATF4)-dependent Pathway to Age-related Skeletal Muscle Weakness and Atrophy, J Biol Chem. 2015 Oct 16; 290(42): 25497-25511), GSK2606414, and TRIB3. Other ATF4 inhibitors include compounds disclosed in U.S. Pat. No. 9,034,299, entitled “ATF4 inhibitors and their use for neural protection, repair, regeneration, and plasticity,” by Ratan; United States Patent Application Publication Number 20160317526, entitled, “Prolylhydroxylase/atf4 inhibitors and methods of use for treating neural cell injury or death and conditions resulting therefrom,” by Ratan and Karuppagounder; and PCT International Patent Application Number WO2017212423, entitled “Chemical Compounds,” by Axten et al. Each of these documents is incorporated herein by reference.

ISR node inhibitors further include nucleic acid molecules which bind to the gene, gene product, or transcript of an ISR node and block or reduce the activity, expression, or translation of the same. Examples of ISR node inhibitors include siRNA, shRNA, and antisense oligonucleotides (ASOs).

### x. Specific Inhibitors of VEGF and VEGFR

The phrase “a composition that blocks or inhibits the expression, induction, activity, and/or signaling of one or more of a subtype or isoform of VEGF or a subform of a VEGF receptor” refers to compositions that include without limitation any VEGF antagonist capable of neutralizing, blocking, inhibiting, abrogating, reducing, or interfering with VEGF activities including its binding to one or more VEGFR. Among such inhibitors or antagonists are an anti-VEGF antibody or antibody fragment directed at an epitope of VEGF isotype A through F or a VEGF-binding fragment thereof, an artificial single chain antibody fragment, or a molecule that mimics the VEGF receptor to bind the VEGF isotypes, or a drug that interferes with receptor signaling. For example, the VEGF antagonist can be, without limitation, an anti-VEGF antibody, a VEGF-trap, an anti-VEGFR antibody, a VEGFR inhibitor, thalidomide, a DI 14-Notch inhibitor, an anti-tubulin vascular disrupting agent (VDA), an angiopoietin-Tie2 inhibitor, a nitric oxide synthase (NOS) inhibitor, or a cationic poly amino acid dendrimer. As used herein, the term “VEGF trap” refers to a decoy VEGF receptor that blocks the VEGF signaling pathway by binding preferentially to VEGF thereby inhibiting its binding to its cognate receptors. In one embodiment, the VEGF trap is a recombinant fusion protein comprising one or more extracellular domains of VEGF receptors, or portions of such extracellular domains, fused to a second protein. For example, a VEGF receptor extracellular domain may be fused to an Fc isoform, such as the Fc fragment of an immunoglobulin. In one embodiment, a VEGF trap is the trademarked EYLEA® drug (Regeneron), which is a recombinant fusion protein, consisting of portions of human VEGF receptors 1 and 2 extracellular domains fused to the Fc portion of human IgG1 and formulated as an iso-osmotic solution for intravitreal administration. In another embodiment, a VEGF trap is OPT-302 (Opthea; Dugel, Pravin U. et al., Phase 1 Study of OPT-302 Inhibition of Vascular Endothelial Growth Factors C and D for Neovascular Age-Related Macular Degeneration Ophthalmology Retina, DOI: https://doi.org/10.1016/j.oret.2019.10.008), i.e., the fusion protein comprising immunoglobulin-like domains 1 to 3 of the extracellular domain of VEGFR-3 fused to the Fc fragment of human immunoglobulin G1 (IgG₁). Still other traps involve other receptor domains and other Fc isoforms.

In certain embodiments, specific examples of VEGF inhibitors include, without limitation, rapamycin, everolimus, temserolimus, a low molecular weight heparin, a SPARC (osteonectin) peptide, bevacizumab, ranibizumab, ramucirumab, aflibercept, interleukin 17 (IL-17), DC101, sunitinib, sorafenib, pazopanib, AMG706, cediranib, vandetanib, vargatef, brivanib, XL-184, axitinib, tivozanib, thalidomide, lanalidomide, DMXAA, nadroparin, 2,5-dimethyl-celecoxib, cyclophosphamide, HBC, and tasquinimod.

In certain embodiments the VEGF inhibitor or antagonist is selected from ranibizumab (Lucentis®), bevacizumab (Avastin®), aflibercept (Eylea®), brolucizumab (Boevu®), pegaptanib (Macugen®), Abicipar pegol (see, e.g., Moisseiev, E., Loewenstein, A. Abicipar pegol-a novel anti-VEGF therapy with a long duration of action. Eye (2019). https://doi.org/10.1038/s41433-019-0584-y), the ranibizumab biosimilars FYB201 (Bioeq GMBH, Munich, GE), PF582 (Pfenex Inc.), SB11 (Samsung Bioepis Co. Ltd), and Xlucane (Xbrane Biopharma), the aflibercept biosimilar MYL-1701P/M-710 (Momenta Pharmaceuticals, Inc.), or conbercept (Liu K, et al; PHOENIX study group. Conbercept for treatment of neovascular age-related macular degeneration: Results of the Randomized phase 3 PHOENIX study. Am J Ophthalmol. 2019;197:156-167); faricimab/RG7716 (Mojunder, M I et al, The Mechanism of the Bispecific Antibody Faricimab Retinal Physician, Volume: 16, March 2019, 32-35), OPT-302 (see, Dugel, cited above), KS-301 (Kodiak Sciences; Wykoff C C. Extended durability in exudative retinal diseases using the novel intravitreal anti-VEGF antibody biopolymer conjugate is KSI-301: Results from the Phase 1b study in patients with AMD, DME and RVO. Presented at American Academy of Ophthalmology Subspecialty Day Retina 2019; October 11, 2019; San Francisco, CA.), KS-501 (Kodiak Sciences; see, e.g., https://www.prnewswire.com/news-releases/kodiak-sciences-announces-emerging-durability-data-from-ongoing-phase-1b-study-of-ksi-301-in-wet-amd-patients-at-the-retina-society-annual-meeting-300918262).

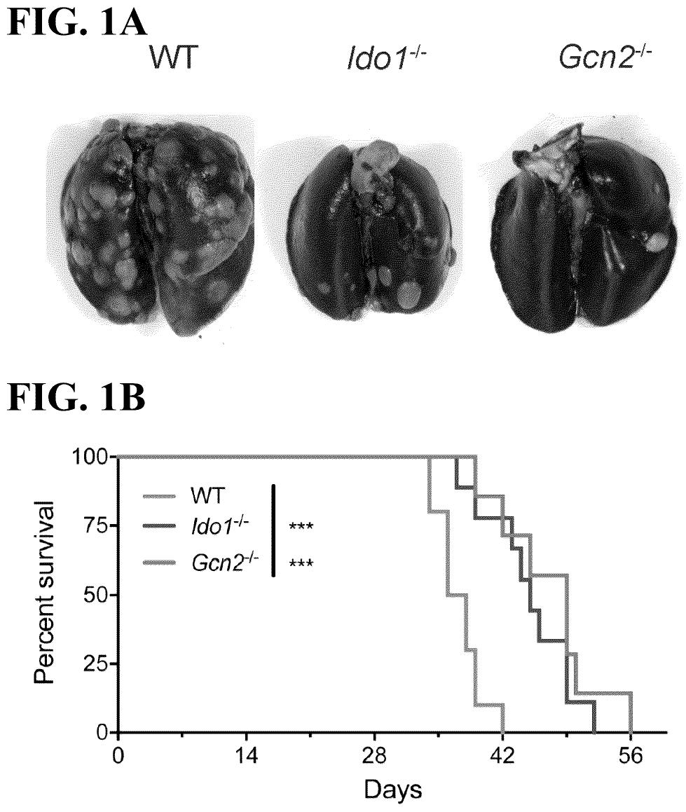

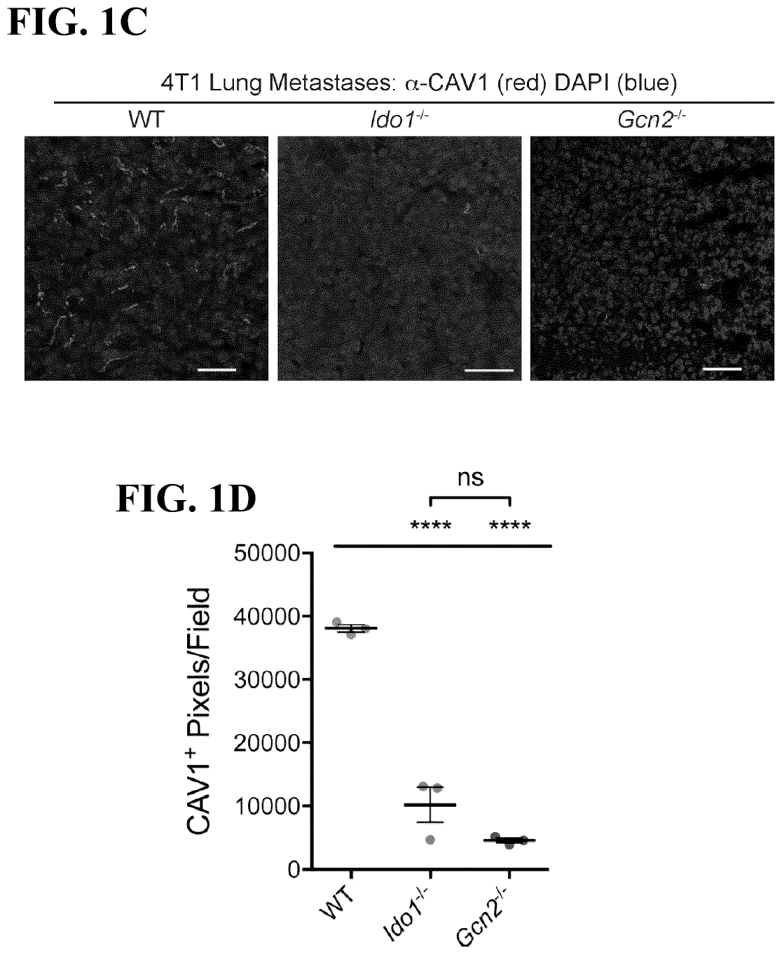

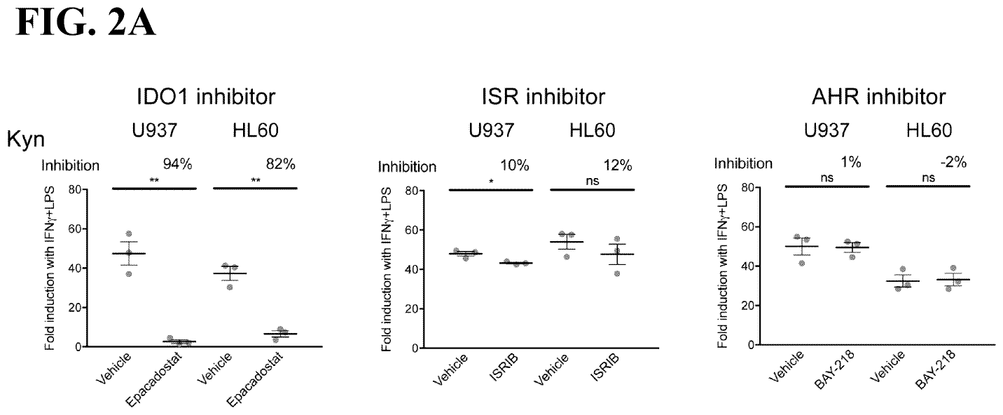

In some embodiments, the VEGF antagonists useful in the combinations are selected from those described in US Patent Publication US20190381087, including ranibizumab (commercially available under the trademark Lucentis® (Genentech, San Francisco, Calif); see FIG. 1 of U.S. Pat. No. 7,060,269 for the heavy chain and light chain variable region sequences), bevacizumab (commercially available under the trademark Avastin®(Genentech, San Francisco, Calif); see FIG. 1 of U.S. Pat. No. 6,054,297 for the heavy chain and light chain variable region sequences), aflibercept (commercially available under the trademark Eylea® (Regeneron, Tarrytown, N.Y.), KH902 VEGF receptor-Fc fusion protein (see Zhang et al. (2008) Mol Vis. 14:37-49), 2C3 antibody (see U.S. Pat. No. 6,342,221, Column 8, lines 48-67, Column 9, lines 1-21), ORA102 (available from Ora Bio, Ltd.), pegaptanib (e.g., pegaptanib sodium; commercially available under the trademark Macugen® (Valeant Pharmaceuticals, Bridgewater, N.J.; see FIG. 1 of U.S. Pat. No. 6,051,698)), bevasiranib (see Dejneka et al. (2008) Mol Vis. 14:997-1005), SIRNA-027 (Shen et al. (2006) Gene Ther. 13:225-34), decursin (see U.S. Pat. No. 6,525,089 (Column 3, lines 5-16)), decursinol (see Ahn et al. (1997) Planta Med. 63:360-1), picropodophyllin (see Economou (2008) Investigative Ophthalmology & Visual Science. 49:2620-6), guggulsterone (see Kim et al. (2008) Oncol. Rep. 20:1321-7), PLG101 (see Ahmadi and Lim (2008) Expert Opin Pharmacother. 9:3045-52), PLG201 (see Ahmadi and Lim (2008)), eicosanoid LXA4 (see Baker et al (2009) J Immun. 182:3819-26), PTK787 (commercially available under the trademark Vitalanib™; see Barakat and Kaiser (2009) Expert Opin Investig Drugs 18:637-46), pazopanib (see Takahashi et al. (2009) Arch Ophthalmol. 127:494-9), axitinib (see Hu-Lowe et al. (2008) Clin Cancer Res. 14:7272-83), CDDO-Me (see Sogno et al. (2009) Recent Results Cancer Res. 181:209-12), CDDO-Imm (see Sogno et al. (2009)), shikonin (see Hisa et al. (1998) Anticancer Res. 18:783-90), beta-hydroxyisovalerylshikonin (see Hisa et al. (1998)), ganglioside GM3 (Chung et al. (2009) Glycobio. 19:229-39), DC101 antibody (see U.S. Pat. No. 6,448,077, Column 2, lines 61-65), Mab25 antibody (see U.S. Pat. No. 6,448,077, Column 2, lines 61-65), Mab73 antibody (see U.S. Pat. No. 6,448,077, Column 2, lines 61-65), 4A5 antibody (see U.S. Pat. No. 6,383,484, Column 12, lines 50-54), 4E10 antibody (see U.S. Pat. No. 6,383,484, Column 10, lines 66-67, Column 11, lines 1-2), 5F12 antibody (see U.S. Pat. No. 6,383,484, Column 10, lines 62-65), VA01 antibody (see U.S. Pat. No. 5,730,977, Column 6, lines 26-30), BL2 antibody (U.S. Pat. No. 5,730,977, Column 6, lines 30-32), VEGF-related protein (see U.S. Pat. No. 6,451,764, FIG. 1), sFLT01 (see Pechan et al. (2009) Gene Ther. 16:10-6), sFLT02 (see Pechan et al. (2009)), Peptide B3 (see Lacal et al. (2008) Eur J Cancer 44:1914-21), TG100801 (see Palanki et al. (2008) J Med Chem. 51:1546-59), sorafenib (commercially available under the trademark Nexavar™; see Kernt et al. (2008) Acta Ophthalmol. 86:456-8), G6-31 antibody (see Crawford et al. (2009) Cancer Cell 15:21-34), ESBA1008 (see U.S. Pat. No. 8,349,322), tivozanib (see U.S. Pat. No. 6,821,987, incorporated by reference in its entirety; Campas et al. (2009) Drugs Fut 2009, 34(10): 793), or a pharmaceutically acceptable salt or prodrug thereof.

In another embodiment, the VEGF antagonist is an antibody or an antibody fragment which binds to an epitope VEGF-A or VEGF-B, or any portion of the epitopes as described in the references cited above and all incorporated by reference. In one embodiment, the VEGF antagonist is an antibody or antibody fragment that binds to one or more of an epitope of VEGF. In another embodiment, the VEGF antagonist is an antibody or an antibody fragment which binds to an epitope of VEGF, such as an epitope of VEGF-A, VEGF-B, VEGF-C, VEGF-D, or VEGF-E. In some embodiments, the VEGF antagonist binds to an epitope of VEGF such that binding of VEGF and VEGFR are inhibited. In one embodiment, the epitope encompasses a component of a three-dimensional structure of VEGF that is displayed, such that the epitope is exposed on the surface of the folded VEGF molecule. In one embodiment, the epitope is a linear amino acid sequence from VEGF.

In some embodiments, an inhibitory antibody directed against VEGF is known in the art, e.g., those described in U.S. Pat. Nos. 6,524,583, 6,451,764 (VRP antibodies), U.S. Pat. Nos. 6,448,077, 6,416,758, 6,403,088 (to VEGF-C), U.S. Pat. No. 6,383,484 (to VEGF-D), U.S. Pat. No. 6,342,221 (anti-VEGF antibodies), U.S. Pat. Nos. 6,342,219 6,331,301 (VEGF-B antibodies), and U.S. Pat. No. 5,730,977, and PCT publications WO96/30046, WO 97/44453, and WO 98/45331, the contents of which are incorporated by reference in their entirety.

Other non-antibody VEGF antagonists include antibody mimetics (e.g., Affibody® molecules, affilins, affitins, anticalins, avimers, Kunitz domain peptides, and monobodies) with VEGF antagonist activity. This includes recombinant binding proteins comprising an ankyrin repeat domain that binds VEGF-A and prevents it from binding to VEGFR-2. One example is MP0112, also known as AGN 150998 (DARPin®). In still other embodiments, recombinant binding proteins comprising an ankyrin repeat domain that binds VEGF-A and prevents it from binding to VEGFR-2 are useful, as described in more detail in WO2010/060748 and WO2011/135067. In still other embodiment, the specific antibody mimetics with VEGF antagonist activity are the 40 kD pegylated anticalin PRS-050 and the monobody angiocept (CT-322).

The aforementioned non-antibody VEGF antagonists may be modified to further improve their pharmacokinetic properties or bioavailability. For example, a non-antibody VEGF antagonist may be chemically modified (e.g., pegylated) to extend its in vivo half-life. Alternatively, or in addition, it may be modified by glycosylation or the addition of further glycosylation sites not naturally present in the protein sequence of the natural protein from which the VEGF antagonist was derived. Another non-antibody VEGF antagonist immunoadhesin currently in pre-clinical development is a recombinant human soluble VEGF receptor fusion protein similar to VEGF-trap containing extracellular ligand-binding domains 3 and 4 from VEGFR2/KDR, and domain 2 from VEGFR1/Flt-1; these domains are fused to a human IgG Fc protein fragment (Li et al., 2011 Molecular Vision 17:797-803). This antagonist binds to isoforms VEGF-A, VEGF-B and VEGF-C. The molecule is prepared using two different production processes resulting in different glycosylation patterns on the final proteins. The two glycoforms are referred to as KH902 (conbercept) and KH906. The fusion protein can be present as a dimer. This fusion protein and related molecules are further characterized in US Patent Publication No. US2010/0215655. All documents reference above for disclosure of suitable VEGF or VEGFr inhibitors or antagonists are incorporated by reference herein.

### xi. Specific Inhibitors of IL-6

Interleukin 6 (IL-6) is a cytokine also called B cell stimulatory factor 2 (BSF2) or interferon β2. IL-6 is a differentiation factor involved in the activation of cells of B lymphocytic series and a multifunctional cytokine that influences the functions of various cells. Various IL-6 inhibitors are known in the art and include, without limitation, IL6 receptor antibodies, including, MH166, SK2, MR16-1, PM-1 antibody, AUK12-20 antibody, AUK64-7 antibody, AUK146-15 antibody, and tocilizumab. Methods of treating choroidal neovascularization by administration of IL-6 inhibitors including anti-IL-6 antibodies, anti-IL-6 receptor antibodies, anti-gp130 antibodies, IL-6 variants, soluble IL-6 receptor variants, and partial peptides of an IL-6 or IL-6 receptor and low molecular weight compounds that show similar activities, are described in U.S. Pat. No. 8,771,686B2, which is incorporated herein by reference.

### H. Effective Amounts, Routes and Related Definitions

A “therapeutically effective amount” of a compound or a pharmaceutical composition refers to an amount effective to prevent, inhibit, treat, or lessen the symptoms of a particular disorder or disease. For example, “therapeutically effective amount” may refer to an amount sufficient to reduce the abnormal vascularization or edema in the eye of a treated subject having the ocular disorder.

A “pharmaceutically acceptable excipient or carrier” refers to, without limitation, a diluent, adjuvant, excipient, auxiliary agent, or vehicle with which an active agent of the present invention is administered. Pharmaceutically acceptable carriers are those approved by a regulatory agency of the Federal or a state government or listed in the U.S. Pharmacopeia or other generally recognized pharmacopeia for use in animals, and more particularly in humans, can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “treatment” refers to any method used that imparts a benefit to the subject, i.e., which can alleviate, delay onset, reduce severity or incidence, or yield prophylaxis of one or more symptoms or aspects of an ocular disease, disorder, or condition. For the purposes of the present invention, treatment can be administered before, during, and/or after the onset of symptoms. In certain embodiments, treatment occurs after the subject has received conventional therapy. In some embodiments, the term “treating” includes abrogating, substantially inhibiting, slowing, or reversing the progression of a condition, substantially ameliorating clinical or aesthetical symptoms of a condition, or substantially preventing the appearance of clinical or aesthetical symptoms of a condition, or decreasing the severity and/or frequency one or more symptoms resulting from the disease.

As used herein, the term “prevent” refers to the prophylactic treatment of a subject who is at risk of developing a condition resulting in a decrease in the probability that the subject will develop the condition.

The term “therapeutic regimen” as used herein refers to the specific order, timing, duration, routes and intervals between administration of one of more therapeutic agents. In one embodiment a therapeutic regimen is subject-specific. In another embodiment, a therapeutic regimen is disease specific. In another embodiment, the therapeutic regimen changes as the subject responds to the therapy. In another embodiment, the therapeutic regimen is fixed until certain therapeutic milestones are met.

In one embodiment, the methods described herein include inhibiting pathologic neovascularization in a subject. The method includes, in one embodiment, ablating or inhibiting IDVCs in the retina of the subject. In another embodiment, the method includes the administration of a composition that blocks or inhibits the expression, induction, activity, and/or signaling of an ISR node. In one embodiment, the ISR node is selected from GCN2, CHOP, and ATF4.

In yet other embodiments, the methods described herein include administering a combination of a composition that blocks or inhibits the expression, induction, activity, and/or signaling of an ISR node; and a composition that blocks or inhibits the expression, induction, activity, and/or signaling of one or more of a subtype or isoform of VEGF or a subtype or isoform of a VEGFR, where the therapeutic regimen involves one or more doses of the administered compositions.

By “therapeutic effect” or “treatment benefit” as used herein is meant an improvement or diminution in severity of a disease system. An “effective amount” is meant the amount of composition sufficient to provide a therapeutic benefit or therapeutic effect after a suitable course of administration.

It should be understood that the “effective amount” for a composition which comprises at least one IDO inhibitor, VEGF or VEGFR inhibitor, or ISR node inhibitor (e.g., GCN2, CHOP or ATF4) will vary depending upon the inhibitor/antagonist selected for use in the method. Regarding doses, it should be understood that “small molecule” drugs are typically dosed in fixed dosages rather than on a mg/kg basis. With an injectable a physician or nurse can inject a calculated amount by filling a syringe from a vial with this amount. In contrast, tablets and some biologics come in fixed dosage forms. Some dose ranging studies with small molecules use mg/kg, but other dosages can be used by one of skill in the art, based on the teachings of this specification. In one embodiment an effective amount for the inhibitor of a composition includes without limitation about 0.001 to about 25 mg/kg subject body weight. In one embodiment, the range of effective amount is 0.001 to 0.01 mg/kg body weight. In another embodiment, the range of effective amount is 0.001 to 0.1 mg/kg body weight. In another embodiment, the range of effective amount is 0.001 to 1 mg/kg body weight. In another embodiment, the range of effective amount is 0.001 to 10 mg/kg body weight. In another embodiment, the range of effective amount is 0.001 to 20 mg/kg body weight. In another embodiment, the range of effective amount is 0.01 to 25 mg/kg body weight. In another embodiment, the range of effective amount is 0.01 to 0.1 mg/kg body weight. In another embodiment, the range of effective amount is 0.01 to 1 mg/kg body weight. In another embodiment, the range of effective amount is 0.01 to 10 mg/kg body weight. In another embodiment, the range of effective amount is 0.01 to 20 mg/kg body weight. In another embodiment, the range of effective amount is 0.1 to 25 mg/kg body weight. In another embodiment, the range of effective amount is 0.1 to 1 mg/kg body weight. In another embodiment, the range of effective amount is 0.1 to 10 mg/kg body weight. In another embodiment, the range of effective amount is 0.1 to 20 mg/kg body weight. In another embodiment, the range of effective amount is 1 to 25 mg/kg body weight. In another embodiment, the range of effective amount is 1 to 5 mg/kg body weight. In another embodiment, the range of effective amount is 1 to 10 mg/kg body weight. In another embodiment, the range of effective amount is 10 to 20 mg/kg body weight. In another embodiment, the range of effective amount is 20 to 30 mg/kg body weight. In another embodiment, the range of effective amount is 30 to 40 mg/kg body to weight. In another embodiment, the range of effective amount is 40 to 50 mg/kg body weight. In another embodiment, the range of effective amount is 1 to 50 mg/kg body weight. Still other doses falling within these ranges are expected to be useful.

An “effective amount” for the composition that blocks or inhibits the expression, induction, activity, and/or signaling of one or more of a subtype or isoform of VEGF or a subtype or isoform of a VEGFR is achieved when the composition comprises at least one VEGF or VEGFR inhibitor and is administered to the subject in an amount suitable to achieve therapeutic effect. These amounts can differ based upon the specific inhibitor chosen and the route of administration. The “effective amount” for the composition which comprises at least one VEGF or VEGFR antagonist or blocker, can vary depending upon the inhibitor/antagonist selected for use in the method. Where the VEGF inhibitor is a small molecule, it may be delivered in the doses the same as described above for a composition comprising at least one ISR node inhibitor (e.g., GCN2, CHOP or ATF4) inhibitor as described above. Where the VEGF antagonist is a protein, e.g., antibody, antibody fragment or recombinant protein or peptide, the effective amount can be similar to that approved for VEGF monotherapy, i.e. 0.01 to 25 mg antibody/injection. In one embodiment, the effective amount is 0.01 to 10 mg antibody/injection. In another embodiment, the effective amount is 0.01 to 1 mg antibody/injection. In another embodiment, the effective amount is 0.01 to 0.10 mg antibody/injection. In another embodiment, the effective amount is 0.2, 0.5, 0.8, 1.0, 1.2, 1.4, 1.6, 1.8, 2.0, 2.2, 2.4, 2.6, 2.8, 3.0 up to more than mg antibody/injection. Still other doses falling within these ranges are expected to be useful.

In one embodiment, the dose and dosage regimen of the composition that is suitable for administration to a particular patient may be determined by a physician considering the patient's age, sex, weight, general medical condition, and the specific condition and severity thereof for which the compositions are being jointly administered. The physician may also consider the route of administration of the agent, the pharmaceutical carrier with which the agent(s) may be combined, and the agent's biological activity.

“Administration” or “routes of administration” include any known route of administration that is suitable to the selected inhibitor or composition, and that can deliver an effective amount to the subject. In one embodiment of the methods described herein, the routes of administration of the compositions are the same. In another embodiment, the compositions are administered by different routes than each other. Routes of administration useful in the methods of this invention include one or more of oral, parenteral, intravenous, intra-nasal, sublingual, intraocular injection, intravitreal injection, via a depot formulation or device, via eye drops, by inhalation. In one embodiment, an inhibitor may be administered orally, or potentially administered intravitreally at the same time as another inhibitor, or potentially combined with the another inhibitor therapeutic in the same device (syringe or depot device), or administered through eye drops, intranasally or sub-lingually.

Throughout this specification, the words “comprise”, “comprises”, and “comprising” are to be interpreted inclusively rather than exclusively. The words “consist”, “consisting”, and its variants, are to be interpreted exclusively, rather than inclusively. It should be understood that while various embodiments in the specification are presented using “comprising” language, under various circumstances, a related embodiment is also described using “consisting of” or “consisting essentially of” language.

The term “a” or “an”, refers to one or more, for example, “a biomarker,” is understood to represent one or more biomarkers. As such, the terms “a” (or “an”), “one or more,” and “at least one” are used interchangeably herein.

As used herein, the term “about” means a variability of 10% from the reference given, unless otherwise specified.

### II. Pharmaceutical Preparations

Compositions described herein may be contained in a single composition comprising at least one carrier (e.g., pharmaceutically acceptable carrier). Alternatively, the agents may be administered separately (e.g., administered in separate compositions) comprising at least one carrier. The pharmaceutical preparation comprising the compositions may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of the agents in the chosen medium may be varied and the medium may be chosen based on the desired route of administration of the pharmaceutical preparation. Except insofar as any conventional media or agent is incompatible with the inhibitors or compositions to be administered, its use in the pharmaceutical preparation is contemplated.

In a particular embodiment, when more than one composition is administered, the compositions may be administered sequentially and/or concurrently. For example, a composition containing an ISR node inhibitor may be administered before, after, and/or at the same time as a composition containing the anti-VEGF inhibitor or antagonist. When the compositions are not administered at the same time, the compositions should be administered close enough in time such that the two or more of the compositions are capable of acting synergistically, additively, or in a manner to achieve a treatment benefit in the subject.

Selection of a suitable pharmaceutical preparation depends upon the method of administration chosen. For example, the composition(s) may be administered by direct injection into the eye or a specific tissue of the eye. In this instance, a pharmaceutical preparation comprises the agent(s) dispersed in a medium that is compatible with ocular delivery. Agents may also be administered parenterally by intravenous injection into the blood stream, or by subcutaneous, intramuscular or intraperitoneal injection. Pharmaceutical preparations for parenteral injection are known in the art. If parenteral injection is selected as a method for administering the antibodies, steps must be taken to ensure that sufficient amounts of the molecules reach their target cells to exert a biological effect. The lipophilicity of the agents, or the pharmaceutical preparation in which they are delivered, may be increased so that the molecules can better arrive at their target locations.

Pharmaceutical compositions containing the inhibitors/antagonists described herein as the active ingredient in intimate admixture with a pharmaceutical carrier can be prepared according to conventional pharmaceutical compounding techniques. The carrier may take a wide variety of forms depending on the form of preparation desired for administration. In preparing the agent in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric coated by standard to techniques. For parenteral compositions, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of the compositions of the invention may be determined by evaluating the toxicity of the active therapeutic inhibitor in animal models. Various concentrations of the above-mentioned inhibitors including those in combination may be administered to a mouse model of an ocular disease (such as oxygen-induced retinopathy (OIR)), and the minimal and maximal dosages may be determined based on the results of significant reduction of vascularization or edema and side effects as a result of the treatment. Appropriate dosage unit may also be determined by assessing the efficacy of the inhibitor compositions in combination with other standard drugs for treatment of ocular disorders. The dosage units of the inhibitors may be determined individually or in combination with each ocular treatment a selected symptom.

The compositions comprising the combined inhibitors of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### III. Specific Methods and Compositions

The methods provided herein relate to the inventors' identification of IDVCs and their role in pathologic neovascularization associated with ocular disease. Provided herein in one embodiment, is a method of treating retinopathy or inhibiting pathologic neovascularization in a subject, the method comprising ablating or inhibiting the activity of IDO-dependent vascularizing cells (IDVCs) in the eye of the subject. IDVCs are functionally characterized as having a role in neovascularization, the establishment and maintenance of which requires the induction of IDO1 within the IDVCs.

In one embodiment, the IDVCs are inhibited by inhibiting the activity of IDO1. IDO1 inhibitors are described herein.

In another embodiment, the IDVCs are inhibited via an antibody directed to a cell-surface marker of the IDVCs. In one embodiment, the antibody is an ADC, or antibody-drug conjugate. The cell surface marker may be selected from any that distinguishes the IDVCs from another non-targeted cell population. For example, in one embodiment, the cell surface marker is selected from CD33, CD11b, CD15, and CD66.

In another embodiment, the method includes blocking or inhibiting the Integrated Stress Response (ISR) in the IDVC cells within the tissues of the eye (e.g. the retina or the choroid). The inventors have shown that inhibition of key ISR nodes, such as GCN2, CHOP, and ATF4, reduced neovascularization in an OIR mouse model. Thus, in one embodiment, the method includes blocking or inhibiting the expression, induction, activity, and/or signaling of an ISR pathway node. Inhibitors of ISR pathway nodes include small molecules, biologic molecules, and nucleic acid molecules that inhibit the translation or transcription of said ISR pathway node. These molecules may be those known in the art, or those to be discovered. Non-limiting examples are described herein.

In one embodiment, the method includes blocking or inhibiting the expression, induction, activity, or signaling of GCN2. In one embodiment, the method includes blocking or inhibiting the expression, induction, activity, or signaling of ATF4. In one embodiment, the method includes blocking or inhibiting the expression, induction, activity, or signaling of CHOP. In one aspect, the method further includes blocking or inhibiting IL-6.

In one embodiment, the method includes administering an effective amount of GCN2-IN-1 (A-92). In another embodiment, the method includes administering an effective amount of GCN2iA. In another embodiment, the method includes administering an effective amount of GZD824. In another embodiment, the method includes administering an effective amount of an inhibitor based on a triazolo[4,5-d]pyrimidine scaffold such as those described by Lough et al (Triazolo[4,5-d]pyrimidines as Validated General Control Nonderepressible 2 (GCN2) Protein Kinase Inhibitors Reduce Growth of Leukemia Cells, Volume 16, September 2018, Pages 350-360).

In another embodiment, the method includes administering an effective amount of an oligonucleotide such as those described by Klar et al (Abstract 3275: Inhibition of ER-stress factor C/EBP homologous protein (Chop) with LNAplus™ antisense-oligonucleotides to improve immunotherapy of cancer, DOI: 10.1158/1538-7445.AM2019-3275 Published July 2019). In another embodiment, the method includes administering an effective amount of ISRIB.

In another embodiment, the method includes administering an effective amount of ursolic acid. In another embodiment, the method includes administering an effective amount of tomatidine. In another embodiment, the method includes administering an effective amount of GSK2606414. In another embodiment, the method includes administering an effective amount of TRIB3.

In another aspect, a method of treating retinopathy or inhibiting pathologic neovascularization in a subject is provided. The method includes blocking or inhibiting signaling molecules downstream of the Integrated Stress Response. Certain signaling molecules downstream of the ISR are known and include the cytokine IL-6.

Another embodiment of the recited methods further includes blocking or inhibiting the expression, induction, activity, or signaling of any form of vascular endothelial growth factor (VEGF). In one embodiment, the VEGF inhibitor comprises one or more of: ranibizumab (Lucentis®), bevacizumab (Avastin®), aflibercept (Eylea®), brolucizumab (Boevu®), pegaptanib (Macugen®), Abicipar pegol, the ranibizumab biosimilars FYB201, PF582, SB11, and Xlucane, the aflibercept biosimilar MYL-1701P/M-710, or conbercept, faricimab/RG7716 (bispecific antibody VEGF-A+Ang-2), OPT-302 (VEGF-C/D ‘trap’), KS301 (Kodiak Sciences—anti-VEGF polymer conjugated biologic), KS501 (Kodiak Sciences—anti-VEGF trap plus anti-IL6 Antibody Fusion).

Another embodiment of the recited methods further includes blocking or inhibiting the expression, induction, activity, or signaling of any form of indoleamine 2,3 dioxygenase-1 (IDO1). In one embodiment, the blocker or inhibitor of IDO-1 comprises at least one of: 1-methyl-D-tryptophan (indoximod), 1-methyl-L-tryptophan, a racemic mixture of 1-methyl-D-tryptophan and 1-methyl-L-tryptophan, epacadostat, navoximod (GDC-0919), and NLG802, or a salt, enantiomer or pro-drug thereof 1-R-D-tryptophan or 1-R-L-tryptophan, wherein R is a C1-C12 alkyl; methylthiohydantoin-DL-tryptophan (MTH-Trp), β-(3-β)-DL-alanine, β-(3-benzo(b)thienyl)-DL-alanine, 6-nitro-L-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-DL-tryptophan, 5-bromoindoxyl diacetate, Naphthoquinone-based, S-allyl-brassinin, S-benzyl-brassinin, 5-Bromo-brassinin, Phenylimidazole-based, 4-phenylimidazole, Exiguamine A, and NSC401366; or BMS-986205/ONO-7701, PF-06840003/EOS200271, MK-7162/IOM2983, LY3381916, KHK2455, HTI-1090/SHR9146, DN1406131, RG70099, Roxyl-WL, TPST-8844, Ethyl pyruvate, Amg-1 or DX-03-12, or a salt, enantiomer or pro-drug or any therapeutically effective formulation thereof.

In another embodiment, the ISR pathway-blocking drug is selected from: GSK-2606414, RPT-GCN2i, AMG-PERK44, and trans-ISRIB.

Provided herein are certain combination therapies. For example, in one embodiment, an ISR node inhibitor is administered with an IL-6 inhibitor. In another embodiment, an ISR node inhibitor is administered with a VEGF inhibitor. According to this combined therapy treatment method, the subject receives one or more advantageous therapeutic or treatment benefits. Among these are a therapeutic effect or treatment benefit that is enhanced relative to the administration of a single agent. Another advantage is production of a synergistic therapeutic effect or treatment benefit. Still another benefit of the combined therapy is improved tolerability of one or more of the administered compositions.

In certain embodiments, the compositions described herein are administered in a coordinated therapeutic regimen. Based on the selection of the compound included in the composition (i.e. the inhibitors of ISR nodes, IL-6, IDO, VEGF/VEGFR, etc. identified herein) the routes of administration of may be the same and/or the two compositions may be administered in a single formulation. In certain embodiments, compositions described herein are administered sequentially. In certain embodiments, compositions described herein are administered simultaneously. In certain embodiments, the routes of administration of the compositions are the same. In certain embodiments, the routes of administration of the compositions are different. In certain embodiments, the routes of administration comprise at least one route which is oral administration, intravenous injection, intra-nasal administration, sublingual administration, intravitreal injection, intra-ocular injection, administration via a depot formulation or device, or administration via eye drops.

Another benefit of the combined therapy is that the combined administration of inhibitors permits use of a dosage amount of any one administered inhibitor that is lower than the dose approved for single agent use. Alternatively, the combined administration of inhibitors use a dosage amount for two or more administered inhibitors that is lower than the dose approved for single agent use. Yet another benefit is that the co-administration of compound described herein enhances the duration of the therapeutic effect or treatment benefit achieved with any one composition administered alone. For example, in one embodiment, a combination therapy permits a VEGF inhibitor or antagonist to be administered by intraocular injection in a therapeutic regimen that involves at least 5% to at least 20% less frequently than a VEGF inhibitor or antagonist would be administered as a sole therapeutic agent. In another embodiment, the combination therapy permits a VEGF inhibitor or antagonist to be administered by intraocular injection in a therapeutic regimen that is at least 5%, 6%, 7%, 8%, 9%, 10%, 11%, 12%, 13%, 14%, 15%, 16%, 17%, 18%, 19% up to 20% or more less frequently than a VEGF inhibitor or antagonist would be administered as a sole therapeutic agent.

In one embodiment, the IDVCs are located in the retina of the eye. In another embodiment, the IDVCs are located in the choroid of the eye. In yet another embodiment, a method of this invention provides for the use of a composition in which the active therapeutic inhibitor or antagonist penetrates the retina and partitions to the tissues of the eye and achieves an ocular concentration greater than the concentration in said subject's serum.

In any of the methods above, one or more of the compositions further comprises a pharmaceutically acceptable excipient or carrier. Formulations for any of the compositions can be designed depending on the selection of the active inhibitor or agent and the route of administration and dosages.

### IV. Kits

The present invention also includes pharmaceutical kits useful, for example, in the treatment or prevention of ocular diseases or disorders referred to herein which include one or more containers containing a pharmaceutical composition including an ISR node inhibitor in a therapeutically effective amount or for administration according to a desired therapeutic regimen. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

Measures of improvement due to the use of these methods can be determined by evaluation of visual acuity, measurement of retinal fluid and/or vascular leakage, and reduction of side effects due to administration of the therapy. Components that permit these efficacy studies can also be included in the kits.

The following examples are provided to illustrate various embodiments of the present invention. The Examples are not intended to limit the invention in any way.

### EXAMPLE 1—MATERIALS AND METHODS

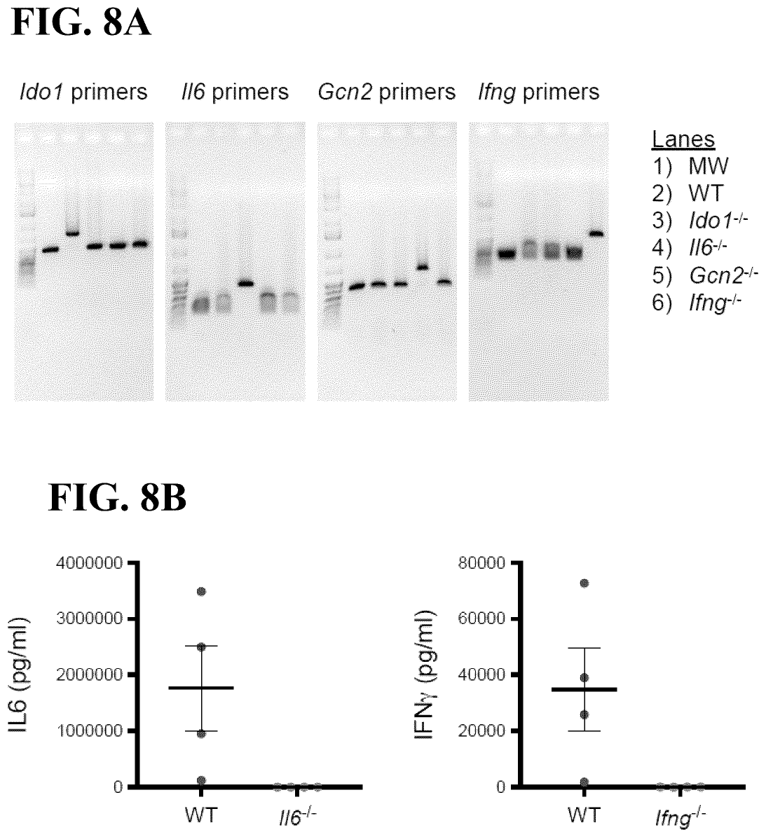

Transgenic mouse strains. Congenic Idol nullizygous (Ido1^{-/-}) mice on the BALB/c strain background were provided by A. Mellor. Congenic Ifng and Il6 nullizygous mice on the BALB/c strain background and WT BALB/c mice were acquired from the Jackson Laboratory. Gcn2 nullizygous mice, established on the 129svev (Taconic) strain background by D. Ron (15), were obtained from A. Mellor and backcrossed for 10 generations in house to obtain a congenic BALB/c strain. Genotyping was performed using specific primer sets designed to distinguish between transgenic and wild type alleles (Tables 1-3) all of which yielded the predicted banding patterns (FIG. 8A).

TABLE 1
List of siRNAs*

| | |
|---|---|
| 18S rRNA | 5′-CAATTACAGGGCCTCGAAAG-3′ |
| | 5′-AAACGGCTACCACATCCAAG-3′ |
| hArf4 | 5′-TCCCATCTCCAGGTGTTCTC-3′ |
| | 5′-CAGCTCTTTGCACTCACCAG-3′ |
| hChop | 5′-GCGCATGAAGGAGAAAGAAC-3′ |
| | 5′-AAAGCTGCGCAGAATGAGATG-3′ |
| hIl6 | 5′-AGTTCCTGCAGAAAAAGGCAAAG-3′ |
| | 5′-AAAGCTGCGCAGAATGAGATG-3′ |

*SEQ ID NOs: 1-8 are listed in descending order

TABLE 2
List of DNA primers*

| Genotyping primers | Sequence |
|---|---|
| Gcn2^{-/-} mice | Mutant reverse (01MR8791) 5′-TGCCACTGTCAGAATCTGAAGCAGG-3′ |
| | Forward (oIMR8796) 5′-TCTCCCAGCGGAATCCGCACATCG-3′ |
| | Reverse (oIMR8797) 5′-ATCCAGGCGTTGTAGTAGCGCAGA-3′ |
| Il6^{-/-} mice | Common forward (oIMR0212) 5′-TTCCATCCAGTTGCCTTCTTGG-3′ |
| | WT reverse (oIMR0213) 5′-TTCTCTTTCCACGATTTCCCAG-3′ |
| | Mutant reverse (oIMR0214) 5′-CCGGAGAACCTGCGTGCAATCC-3′ |
| Ifnγ^{-/-} mice | WT forward (ofMR8284) 5′-AGAAGTAAGTGGAAGGGCCCAGAAG-3′ |
| | Mutant forward (oIMR6218) 5′-CCTTCTATCGCCTTCTTGACG-3′ |
| | Common reverse (oIMR8285) 5′AGGGAAACTGGGAGAGGAGAAATAT-3′ |
| Ido1^{-/-} mice | Common Forward 5′-GGTGGAGCTGCCCGACGC-3′ |
| | WT reverse 5′-TTCCACCACCACCGAGTTAC-3′ |
| | Mutant reverse 5′-AATGCGCTCAGGTCAAATTC-3′ |

*SEQ ID NOs: 9-20 are listed in descending order

TABLE 3
In vivo siRNA

| In Vivo siRNA | Source | Catalog No. |
|---|---|---|
| SMARTpool Accell Ahr siRNA mouse | Dharmacon | E-044066-00-0005 |
| SMARTpool Accell Atf4 siRNA mouse | Dharmacon | E-042737-00-0005 |
| SMARTpool Accell Ddit3 siRNA mouse (Chop) | Dharmacon | E-062068-00-0005 |
| SMARTpool Accell Elf2ak4 siRNA mouse (Gen2) | Dharmacon | E-044353-00-0005 |
| Accell Non-targeting Control Pool | Dharmacon | D-001910-10-05 |

Lack of IDO1 protein expression in the Ido1^{-/-} strain mice has been previously validated (9,16). Lack of IL6 induction in the Il6^{-/-} strain and IFN-γ induction in the Ifng^{-/-} strain was confirmed by assaying lung biopsies at 24 hours following nasal instillation of 25 μg LPS (FIG. 8B). All studies involving mice were approved by the Lankenau Institute for Medical Research IACUC and conform with AAALAC guidelines.

4T1 Tumor Cell Metastasis. Primary orthotopic 4T1 mouse carcinoma tumors were established by injecting 1×10⁴ cells in 50 μl serum free medium into the mammary fatpad of ˜8 week-old female BALB/c mice, which subsequently metastasized to the lungs. Pulmonary metastasis burden was visualized by inflating the lungs with 15% India ink dye (reconstituted with 1× PBS and one drop of ammonium hydroxide), washed, and bleached in Fekete's solution (40 ml of glacial acetic acid, 32 ml of (37%) formalin, 700 ml of (100%) ethanol stock reconstituted with ddH₂O to make 1 liter). For immunohistochemistry analysis, lungs bearing 4T1 metastases were inflated with 50% OCT and frozen in OCT blocks followed by 4 μm sectioning using the CryoJane tape transfer system. Antibodies and staining reagents are listed in Table 4.

TABLE 4
List of Antibodies

| | Source | Catalog No. |
|---|---|---|
| FACS/Cell sorting/Neutralizing antibodies | | |
| APC anti-mouse CD45 antibody, clone 30-F11 | Biolegend | 103112 |
| PerCP anti-mouse Ly-6G/Ly-6C (Gr-1) antibody, clone RB6-8C5 | Biolegend | 108426 |
| FITC anti-mouse/human CD11b, clone M1/70 | Biolegend | 101206 |
| PE/Cy7 anti-mouse/human CD11b antibody, clone M1/70 | Biolegend | 101215 |
| PE anti-mouse CD11c antibody, clone N418 | Biolegend | 117307 |
| Alexa Fluor ® 647 anti-Asialo-GM1 Antibody, clone Poly 21460 | Biolegend | 146004 |
| Biotin anti-mouse CD3ε, clone 145-2C11 | Biolegend | 100303 |
| Biotin anti-mouse CD31, clone MEC13.3 | Biolegend | 102503 |
| Biotin anti-mouse B220, clone RA36B2 | Biolegend | 103204 |
| Biotin anti-mouse CD119 (IFN-γ R α chain), clone 2E2 | Biolegend | 112803 |
| Biotin anti-mouse CD274 (PD-L1), clone 10F.9G2 | Biolegend | 124305 |
| Biotin anti-mouse CD11c, clone N418 | Biolegend | 117303 |
| Biotin anti-mouse I-A/I-E (MHCII), clone M5/114.15.2 | Biolegend | 107603 |
| Biotin anti-mouse CD170(Siglec-F), clone S17007L | Biolegend | 155512 |
| APC anti-mouse F4/80, clone BM8 | Biolegend | 123115 |
| InVivoPlus anti-mouse Ly6G/Ly6C (Gr-1), clone RB6-8C5 | Bioxcell | BP0075 |
| IHC antibodies/reagents | | |
| Caveolin-1 Antibody | Cell Signaling Tech | 3238S |
| Isolectin GS-IB₄ From Griffonia simplicifolia, Alexa Fluor ™ | ThermoFisher | I21411 |
| 488 Conjugate | Scientific | |
| Anti-IDO-1 Antibody, clone 487 | Millipore Sigma | MABF850 |
| FITC anti-mouse CD45 antibody, clone 30-F11 | Biolegend | 103107 |
| Ultra-LEAF ™ Purified anti-mouse Ly-6G/Ly-6C (Gr-1) | Biolegend | 108435 |
| antibody, clone RB6-8C5 | | |
| Biotin anti-mouse/human CD11b antibody, clone M1/70 | Biolegend | 101204 |
| Goat anti-mouse secondary antibody Cy3 conjugated | Life Science | M30010 |
| | Technologies | |
| Goat anti-rabbit FITC and Cy3 | Thermo Fischer | F0257 & |
| | Scientific | A10520 |
| Goat anti-rat FITC | eBioscience | 11-4811-85 |
| Streptavidin Alexa 488 | Biolegend | 405235 |
| ProLong ™ Gold antifade reagent with DAPI | ThermoFisher | P36935 |
| | Scientific | |

Analysis of vasculature in pulmonary metastatic lesions was performed by immunofluorescence staining with rabbit anti-Caveolinl polyclonal antibody. Vessel density within the metastatic nodules was quantified by acquiring multiple fields per mouse lung on a Zeiss inverted microscope with 40× objective and 1.0 optovar. The pixel density corresponding to the positive CAV1 signal per field was determined in Adobe Photoshop, and these values were then averaged to determine an overall mean value per mouse. To examine protein expression in the 4T1 lung metastatic nodules, slides were blocked in 40 μg/mL goat anti-mouse IgG-Fab (H+L) (Jackson ImmunoResearch) followed by 10% normal goat serum (Jackson ImmunoResearch) and immunostained with α-IDO1 (clone 4B7), α-CAV1, α-CD45 (FITC conjugated), α-Gr-1 or α-CD11b (biotinylated) antibodies. Slides were washed and incubated with goat anti-mouse secondary antibody (Cy3 conjugated) to detect IDO1, goat anti-rabbit secondary antibody (FITC or Cy3 conjugated) to detect CAV1, goat anti-rat (FITC conjugated) to detect Gr-1 and streptavidin (Alexa 488 conjugated) to detect CD11b. Subsequently, slides were mounted using Prolong Gold with DAPI (Life Technologies; Cat#P36935) and imaged under confocal microscopy (Nikon Eclipse Ti) with 60× objective using NIS element AR software.

Oxygen-Induced Retinopathy. The OIR model of neovascularization was performed as described (17). Neonatal mice were housed in a chamber with 75% oxygen (OxyCycler) from day 7 to day 12 postpartum (P7-P12). Neonates were removed from the chamber at P12 and were subsequently maintained under normoxic conditions for 5 days (P17) at which point neovascularization in the neonates reaches its peak levels. Quantification of retinal neovascularization was performed by the same confocal microscopy analysis method as previously reported (7), with antibodies and reagents used listed in Table 4. Eyes were fixed (4% paraformadehyde and methanol) and isolated retinas were stained with Isolectin B4-Alexa488 and flat mounted. Where indicated, additional IDO1 staining in the retinas was performed following Isolectin B4-Alexa488 staining. Retinas were blocked in 40 μg/mL goat anti-mouse IgG-Fab (H+L) (Jackson ImmunoResearch) followed by 10% normal goat serum (Jackson ImmunoResearch). Anti-mouse IDO1, α-CD45α (FITC conjugated) and α-Gr-1 (FITC conjugated,) were incubated overnight at 4° C. Retinas were washed and incubated with goat anti-mouse secondary antibody (Cy3 conjugated,) to detect IDO1 and then flat mounted. Confocal microscopy (Nikon Eclipse Ti) imaging was performed at 20× objective using NIS element AR software.

Intravitreal injections. Local delivery of siRNA and antibody treatments in OIR studies was carried out by intravitreal injections. Prior to injection, the topical anesthetic Alcaine (0.5% proparacaine hydrochloride ophthalmic solution) was applied to the eyelids. Eyes were injected under a dissecting microscope through the cornea into the vitreous humor with 1 μl siRNA or antibody in PBS using a 33-gauge needle attached to a 2.5 μl glass syringe. For siRNAs (siGcn2, siAtf4, siChop and siAhr; Tables 1-3), a single injection was administered at P14, with the targeted siRNA injected into one eye and the non-targeted control siRNA injected into the contralateral eye. For anti-Gr-1 antibody (BioXCell), a single injection was administered at P14, with the experimental antibody injected into one eye and the isotype control antibody injected into the contralateral eye. All studies were concluded at P17 for evaluation of the impact on retinal neovascularization and were quantitated by confocal microscopy analysis as described above.

Cell culture. The 4T1 (mouse mammary carcinoma; ATCC Cat# CRL-2539) and U937 (human histiocytic lymphoma; ATCC Cat# CRL-1593.2) cell lines were cultured in DMEM supplemented with penicillin, streptomycin, 10% FBS and 55 μM β-mercaptaethanol. The HL60 (human promyelocytic leukemia; ATCC Cat# CCL-240) cell line was maintained in IMDM media supplemented with 10% FCS plus 55 μM β-mercaptaethanol, 2 mM glutamax (Life Technologies), and 50 μg/ml gentamicin. Negative IMPACT I pathogen test results (RADIL) were obtained for the 4T1 cell line prior to its use in mice. No mycoplasma testing was done on the U937 or HL60 cell lines. For 4T1 tumor engraftment studies, cells from the original stock vial were expanded and immediately frozen and each experiment was performed with cells taken directly out of freezing. In vitro experiments were performed with U937 and HL60 cells maintained no longer than passage 6. U937 and HL60 cells (2×10⁶) were stimulated with IFNγ (100 ng/mL; ThermoFisher Scientific, Cat# PHC4031) and LPS (100 ng/mL; E. coli K-235; Cat# L2018) together with either the IDO1 inhibitor Epacadostat (1 μM+0.01% DMSO; Chemietek Cat# CT-EPAC), the integrated stress response inhibitor ISRIB (1 μM+0.01% DMSO; Sigma-Aldrich Cat# SML0843) , the AHR inhibitor BAY-218 (1 μM+0.01% DMSO; Selleckchem Cat# 2162982) or 0.01% DMSO vehicle alone. At 24 hr mRNA was prepared from the treated cells for qPCR-based analysis of gene expression and cell culture media was analyzed for levels of kynurenine to assess IDO1 enzymatic activity.

Viability assay. Cell viability in U937 and HL60 cells was measured by LDI Cell CountEZ™ toxicity, proliferation and survival (TPS) assay kit (LDI1201™) as per the manufacturer's protocol. Both cell lines were plated at a density of 10,000 cells/ well of a 96 well plate in 100 μl DMEM medium with 10% FBS. Assay groups included untreated cells and IFNγ+LPS stimulation with or without Epacadostat, ISRIB, and BAY-218 as per the gene expression analysis. Bortezomib (20 nM; LC Laboratories, Cat# B-1408) was used as positive control for inducing cellular apoptosis. Treated cells were incubated at 37° C. in a humidified CO2 incubator for 24 hrs. The TPS assay was then performed with cell viability assessed by reading absorbance at 412 nm in a microtiter plate reader.

Kynurenine assay. Levels of kynurenine in U937 and HL60 cell supernatants were measured using Ehrlich's reagent (p-dimethylamino-benzaldehyde). Following treatments, supernatants were collected and transferred to a V-bottom 96-well plate and mixed with 40 μL 50% (w/v) trichloroacetic acid (TCA; LabChem) to precipitate any protein. The plate was then incubated at 65° C. for 30 minutes followed by centrifugation at 1250×g for 10 minutes. Following centrifugation, 100 μL of clarified supernatant was transferred to a new flat-bottomed 96-well plate and mixed at equal volumes with 2% (w/v) Ehrlich's reagent in acetic acid. The resulting reaction was measured at 490 nm using a Synergy HT microtiter plate reader (Bio-Tek). Vitreous humor was collected from eyes of mice that underwent the OIR process. Levels of kynurenine were measured using the IDK® Kynurenin ELISA kit (Immundiagnostik AG, Bensheim, Germany) according to manufacturer's protocol. Additionally, levels of kynurenine were confirmed by high-performance liquid chromatography (HPLC) coupled to electrospray ionization liquid chromatography/ tandem mass spectroscopy (LC/MS-MS) as described (18).

IL6 measurement. To measure the levels of IL6 from the OIR model, vitreous humor from the neonatal retinas was collected immediately following eye harvest. A 1:4 dilution factor of vitreous humor to assay diluent samples was analyzed for the cytokine level using BD Biosciences mouse cytokine bead array IL6 kit. Samples were prepared as per the manufacturer's guidelines. FACS Canto II flow cytometer (BD Biosciences) and FACSDIVA software (BD Biosciences) were used to read the samples and FCAP array Software was used to obtain the concentrations. Cytokine concentrations were calculated by comparing to the standard curve provided by the manufacturer (BD Biosciences) in the cytokine bead array kit.

Quantitative real time PCR analysis. Total cellular RNA was prepared from tissue culture cells using Trizol (Life Technologies) reagent as per manufacturer's protocol. cDNA was prepared using the Transcription First Strand cDNA synthesis kit (Applied biosystems; Cat#4368814). Quantitative PCR (QPCR) reactions were run using the Fast Start Universal SYBR Green Mix (Roche Applied Science) with an Eppendorf Realplex 4 Mastercycler QPCR machine. Levels of mRNA expression detected with primers specific for Atf4, Chop Il6 and Cyp1a1 (Tables 1-3) were normalized to 18S mRNA levels and differences between treatment groups were analyzed using the δ-δ cycle threshold method.

Cell Sorting. Gr-1+CD11b^{lo} and Gr-1+CD11b^{hi} cells were analyzed and sorted using a flow cytometric cell sorter (BD FACSAria III) with antibodies and staining reagents listed in Table 4. At 4.5 weeks (for WT, Ifng^{-/-})—5.5 weeks (Ido1^{-/-}, Il6^{-/-}, Gcn2^{-/-}) after 4T1 injections, tumor bearing lungs were harvested and subsequently dissociated using a gentleMACS Tissue Dissociator (Miltenyi Biotec) as per the manufacturer's guidelines followed by RBC lysis and blocked with Trustain FcxTM (anti-mouse CD16/32) (Biolegend; Cat#101320). From the dissociated lungs, cells were stained for anti-CD45, anti-CD11b and anti-Gr-1 antibodies and then further sorted for Gr-1+ CD11b^{lo} and Gr-1+CD11b^{hi} populations using the FACS Aria III flow cytometer (BD Biosciences). Populations of autofluorescence high (AF^{hi}) and low (AF^{lo}) cells from the Gr-1+ CD11b^{lo} gated population were sorted and levels of autofluorescence were confirmed by confocal microscopy using the 495/519 nm excitation and emission channel. Further sorting was also performed to analyze cells from the Gr-1+ CD11b^{lo} AF^{hi} gated population with either high or low combined CD11c and asialo-GM1 expression (CD11c^{hi} asialo-GM1^{hi} and CD11c^{lo} asialo-GM1^{lo}). To assess IDO1 expression levels in the different sorted cell populations, 50,000 cells were centrifuged onto a slide at 800 rpm using a Shandon Cytospin 3. Slides were dried overnight, fixed with acetone at −20° C. and immunostained for IDO1 (clone 4B7) and subsequently imaged by confocal microscopy (Nikon Eclipse Ti). Cellular morphology of CD11c^{hi} asialo-GM1^{hi} and CD11c^{lo} asialo-GM1^{lo} cells was determined by staining by Differential Quick Staining kit (Modified Giemsa, Electron Microscopy Sciences Cat# 26096-25) according to manufacturer's protocol.

Matrigel Plug Assay. To assess the in vivo neovascularizing capability of select immune cell populations isolated from 4T1 pulmonary metastases, 5×10⁴-2×10⁶ sorted cells were isolated (as indicated in the corresponding figure legends) by fluorescence-activated cell sorting (BD FACSAria III) into RPMI+10% FCS and put on ice. Immediately following sorting, isolated immune cell populations were suspended in 250 μl PBS and combined with 250 μl Matrigel (Corning® Cat# 356237) per plug on ice for in vivo injections. Plugs were established by subcutaneous injection in the dorsal region of recipient mice using an ice-cold syringe with a 21 gauge needle. For studies involving IDO1 inhibition, mice were administered 50 mg/kg epacadostat in 100 μl of vehicle (3% N,N-dimethylacetamide, 10% 1-hydroxypropyl-β-cyclodextrin) by oral gavage b.i.d. for 72 hours beginning 6 days post injection of the Matrigel plug. After 9 days post injection, the matrigel plug was harvested and images recorded with either a Samsung Galaxy S6 or an iPhone X using the 2× lens with an additional 10× macro lens attachment (Moment).

For blood vessel quantification, the Matrigel plugs were blocked in OCT and cryosectioned using the tape transfer method (4≥sections per plug). Following sectioning, neovascularization was visualized under confocal microscope by fluorescence staining with rabbit anti-mouse for caveolin 1 and quantitatively analyzed using Adobe Photoshop as described above.
Statistical Analysis. Statistical analysis and graphing were performed using Prism 7 (GraphPad Software Inc.). Scatter plots are presented with means±SEM and statistical significance was determined by unpaired, 2-tailed Students t-test or by one-way ANOVA with either Dunnett's or Sidak's multiple comparisons test (as indicated in each figure legend). Significance for Kaplan-Meier survival curves was determined by 2-group log-rank test. Ranges of P values are indicated as follows: ****, P<0.0001; ***, P<0.01; **, P<0.01; *, P<0.05: ns, not significant.

### EXAMPLE 2: RESULTS

Genetic loss of GCN2 phenocopies the anti-neovascular effects of both IDO1 loss and IL6 loss

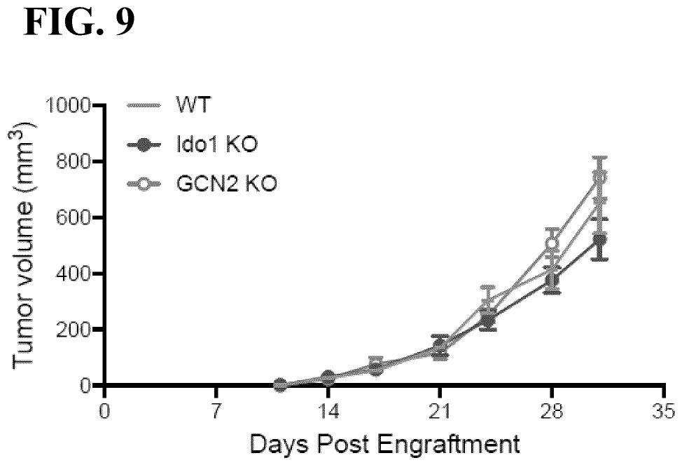

GCN2, a serine kinase activated in response to amino acid deficiency including the catabolism of tryptophan by IDO1, is one of four mammalian kinases that feed into the ISR, which, in turn, has been linked to the regulation of IL6 expression, although there are conflicting reports as to whether the regulatory effect is positive or negative (11,12,19). To probe the biological relevance of this signaling pathway as a possible mechanistic link bridging the IFNγ-dependent effects on neovascularization shared in common by both IDO1 and IL6 (7), we carried out studies in Gcn2^{-/-} mice harboring a targeted disruption of both Gcn2 alleles. Phenotypically Gcn2^{-/-} mice challenged with metastatic 4T1 tumors resembled Ido1^{-/-} mice, exhibiting a primary tumor growth rate similar to that of WT mice (FIG. 9) but markedly reduced metastasis burden in the lungs (FIG. 1A). This metastatic differential was associated with a significant survival advantage that Gcn2^{-/-} mice, like Ido1^{-/-} mice, exhibited relative to WT mice (FIG. 1B). Immunofluorescence staining of 4T1 lung metastases from Gcn2^{-/-} mice revealed evidence of significantly reduced vascular density comparable to that observed in Ido1^{-/-} mice (FIG. 1C and FIG. 1D). These data are consistent with the hypothesis that GCN2 is required for IDO1 to promote tumor growth, neovascularization and IL6 induction in this metastatic setting.

### IDO1 Signals through the Integrated Stress Response Pathway to Induce IL6

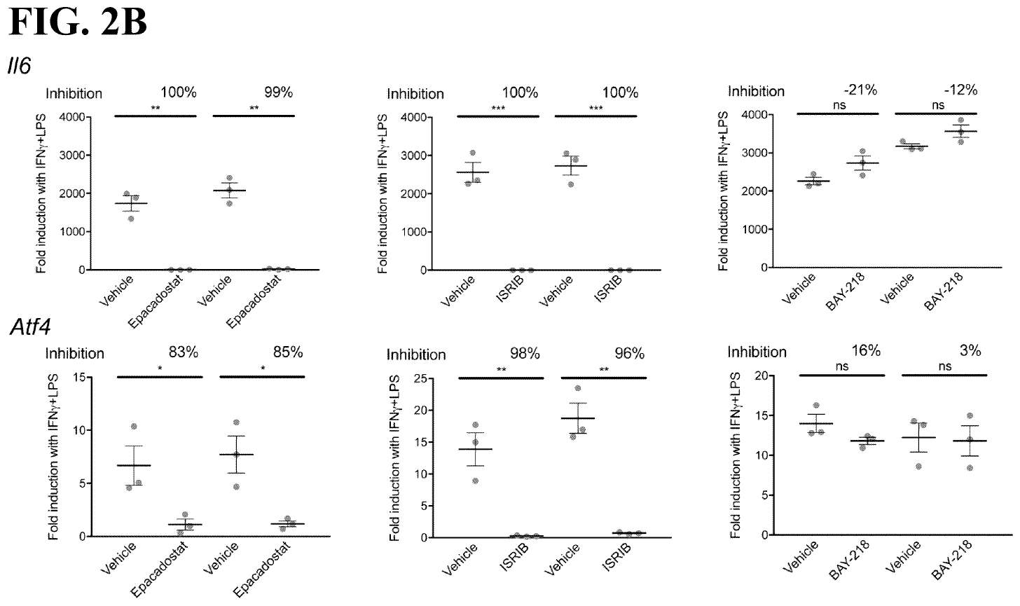

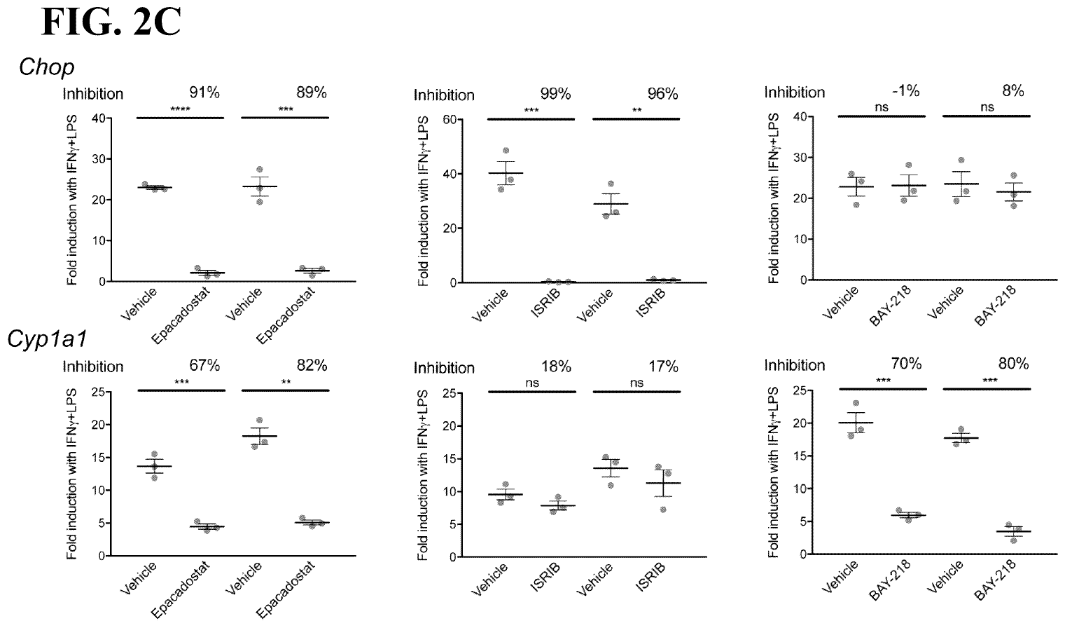

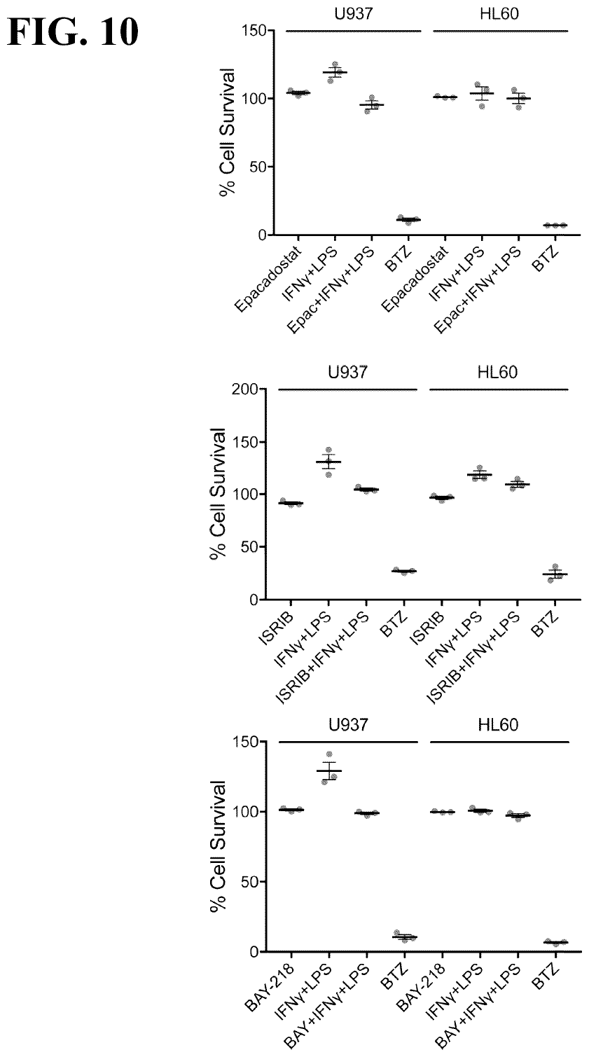

IDO1-mediated tryptophan catabolism can lead to activation of ISR signaling in response to tryptophan depletion and AHR signaling through production of the endogenous ligand kynurenine. To directly investigate if the observed connection between IDO1 activity and IL6 induction is linked through either of these two downstream signaling pathways, we examined the impact of inhibiting both pathways in isolated cell cultures. We have previously shown that IFNγ+LPS-mediated induction of IDO1 activity in both U937 and HL60 cells was accompanied by a corresponding increase in IL6, and that both could be blocked with an IDO1 enzyme inhibitor MTH-Trp (9). In this study we used a different IDO1 inhibitor, Epacadostat, which blocked 80-90% of the kynurenine produced by the cells (FIG. 2A) and resulted in a reduction IL6 gene expression of ≥99% (FIG. 2B-2C), further corroborating the connection between IDO1 activity and IL6 induction. To assess the activation status of the signaling pathways downstream of IDO1, we likewise evaluated genes that are induced upon ISR and AHR activation. CHOP (C/EBP homologous protein/Ddit3 DNA-damage inducible transcript 3) and ATF4 (activating transcription factor 4) are two downstream signaling elements that are induced upon ISR activation (20), while CYP1A1 (cytochrome P450, family 1, subfamily a, polypeptide 1) is a downstream target gene for AHR (21). qPCR analysis revealed elevated levels of CHOP, ATF4, and CYP1A1 message following IDO1 induction, and all three were effectively blocked by Epacadostat treatment (FIG. 2B-2C). To examine which IDO1 signaling pathway might be responsible for controlling IL6 expression, the inhibitors ISRIB, which blocks ISR signaling (22), and BAY-218, which blocks AHR signaling (23), were used. As expected, ISRIB blocked induction of both CHOP and ATF4 but not CYP1A1 while BAY-218 blocked induction of CYP1A1 but not CHOP or ATF4 (FIG. 2B-2C), and neither had a significant effect on the level of kynurenine produced (FIG. 2A). IL6 gene expression was effectively blocked by ISRIB while BAY-218 had no significant effect (FIG. 2B-2C), indicating that, indicative of IL6 induction being controlled by signaling through the ISR and not through the AHR. Neither ISRIB, Epacadostat nor BAY-218 had a sufficient impact on cell viability at the concentrations used to account for the observed effects (FIG. 10). Altogether, these data argue in favor of ISR signaling being the downstream regulatory link that connects IDO1 activity to the induction of IL6.

ISR signaling acts downstream of IDO1 to promote neovascularization in a mouse model of oxygen-induced retinopathy

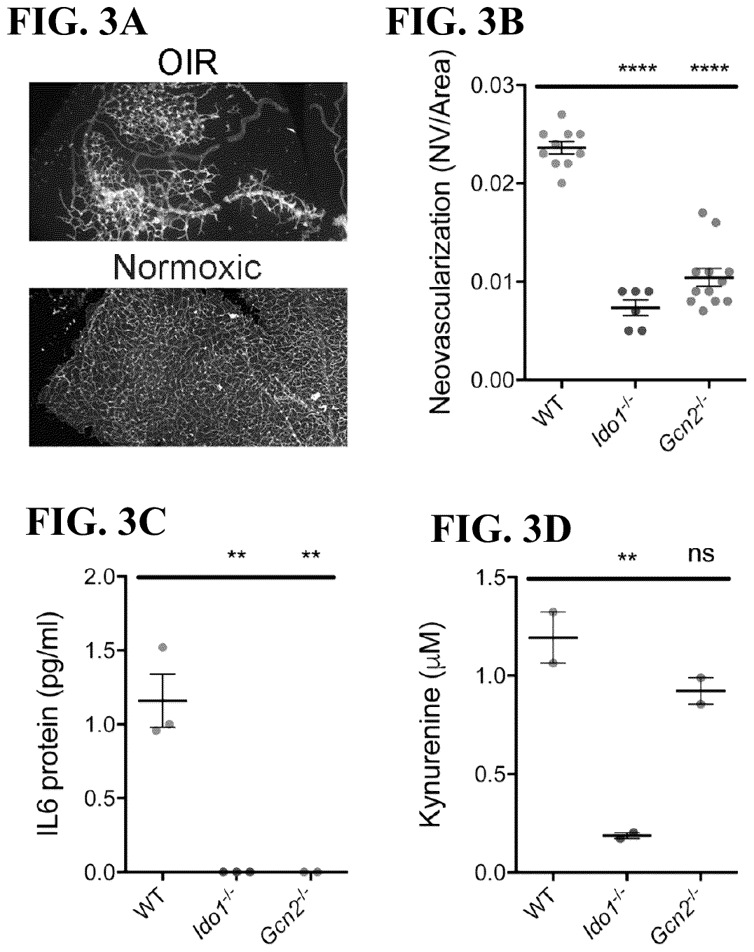

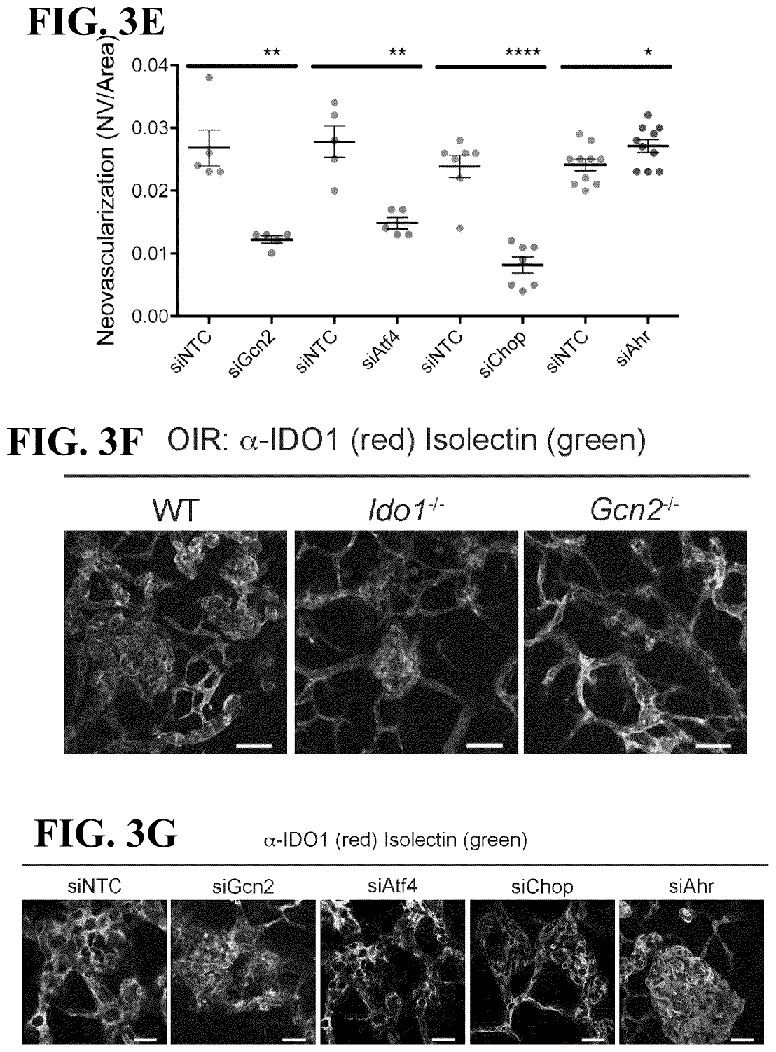

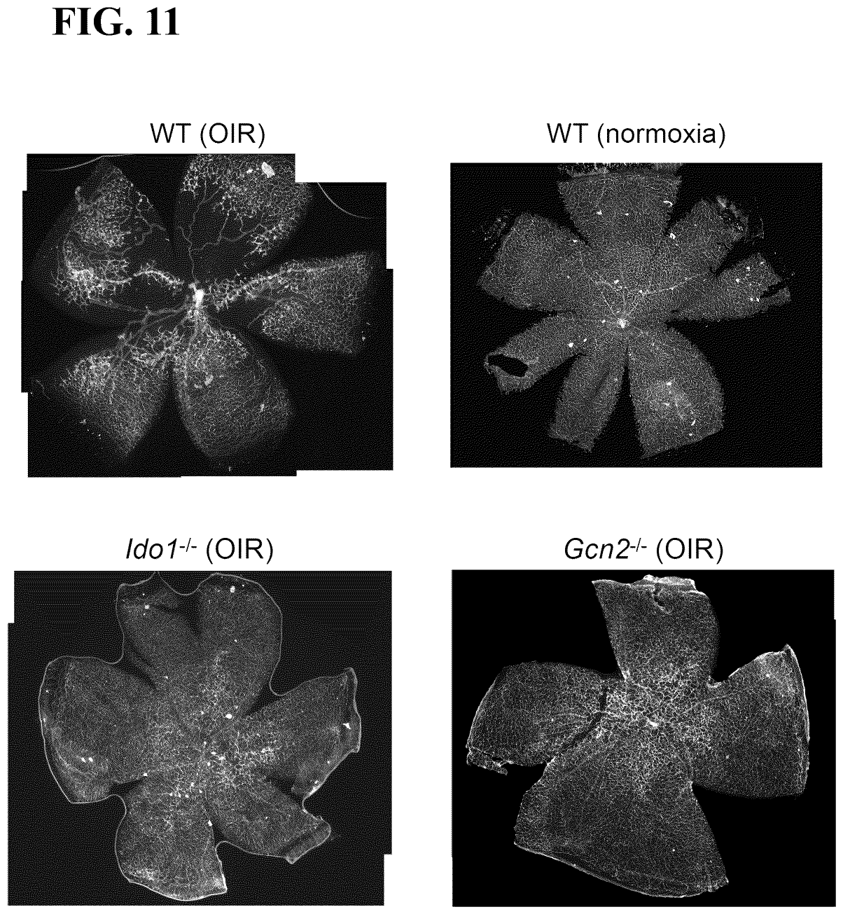

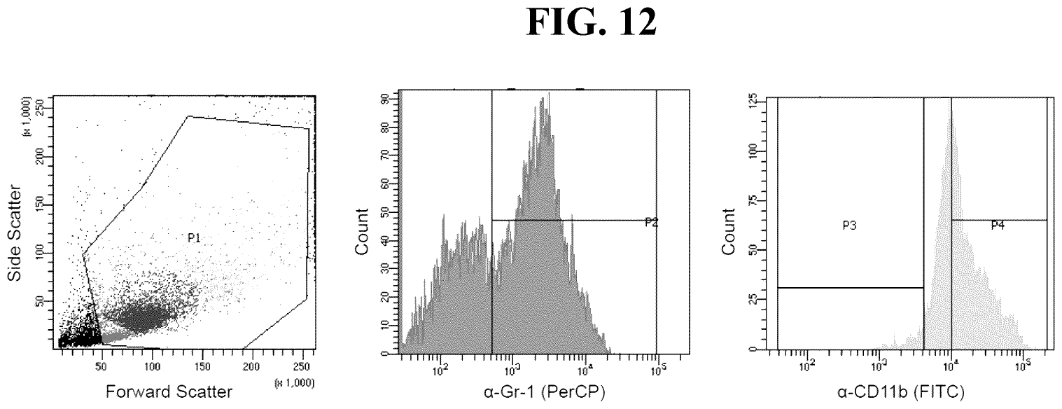

To study the impact of IDO1 on neovascularization apart from its other effects on tumor development and immune escape, we have previously employed a mouse OIR (oxygen-induced retinopathy) model (7) which reproducibly and quantitatively forms abnormal blood vessels (FIG. 3A and FIG. 11) similar to those found in tumors (17). In the OIR model, IDO1 is critical for promoting neovascularization in a manner that counters IFNγ and phenocopies IL6 (7). To evaluate the relevance of ISR signaling in this biological context, OIR associated neovascularization was evaluated in Gcn2^{-/-} mice. Similar to our observation in Ido1^{-/-} mice, neovascularization was found to be significantly reduced in mice lacking GCN2 (FIG. 3B). This effect corresponded with reduced levels of IL6 in the vitreous humor of Gcn2^{-/-} and Ido1^{-/-} mice (FIG. 3C). However, unlike Ido1^{-/-} mice, Gcn2^{-/-} mice did not exhibit significantly reduced levels of kynurenine in the vitreous humor, indicative of IDO1 activity being unaffected in these animals (FIG. 3D).

Additional evidence for the role of ISR signaling in promoting OIR-associated neovascularization came from studies employing specifically directed siRNAs injected into the eyes to target critical signaling components of this pathway, an approach that we previously used to successfully target Ido1 and Vegfa (7). Consistent with the germline knockout data, siRNA mediated targeting of Gcn2 significantly reduced OIR-associated neovascularization (FIG. 3E). Likewise, targeting the ISR signaling components Atf4 or Chop in this manner resulted in similar reductions in OIR-associated neovascularization relative to non-targeted control siRNA injected into the contralateral eye. In contrast, no reduction in OIR-associated neovascularization was observed using siRNAs directed against Ahr to interfere with this alternative IDO1 signaling pathway. Instead, Ahr targeting was associated with a relatively small but significant increase in neovascularization (FIG. 3E) suggesting that AHR signaling may exert a minor counterregulatory effect in this context consistent with the trend towards increased Il6 expression with AHR inhibition in the U937 and HL60 cell-based assays.

In conjunction with immunofluorescence staining of blood vessels, retinal whole mounts were also stained for IDO1. IDO1 expression was detected exclusively in cells closely associated with the neovascular tufts (FIG. 3F). No IDO1 staining was observed outside the neovascular regions in OIR mouse retinas nor anywhere throughout the retinas of mice maintained under constant normoxic conditions (data not shown). No positive staining for IDO1 was detected in the neovascular tufts from Ido1^{-/-} OIR mouse retinas, confirming the specificity of the anti-IDO1 antibody staining (FIG. 3F). In contrast, IDO1 positive staining cells were detectable in OIR retinas from Gcn2^{-/-} mice despite their exhibiting a reduced level of neovascularization comparable to that observed in Ido1^{-/-} mice (FIG. 3F). This observation is consistent with GCN2 acting as a critical signaling component of the ISR signaling pathway downstream of IDO1 and argues against the concern that the lack of IDO1 positive staining observed in Ido1^{-/-} mouse retinas might be a spurious consequence of reduced neovascularization. Likewise, IDO1 expression was detected in the OIR retinas of mice treated with siRNAs to Gcn2, Chop and Atf4, as well as Ahr (FIG. 3G). Taken together, these data are consistent with the ISR being the biologically relevant signaling pathway through which IDO1 acts to promote pathological neovascularization in OIR.

### Expression of IDO1 in a Gr-1+ CD11b^{lo} Immune Cell Population

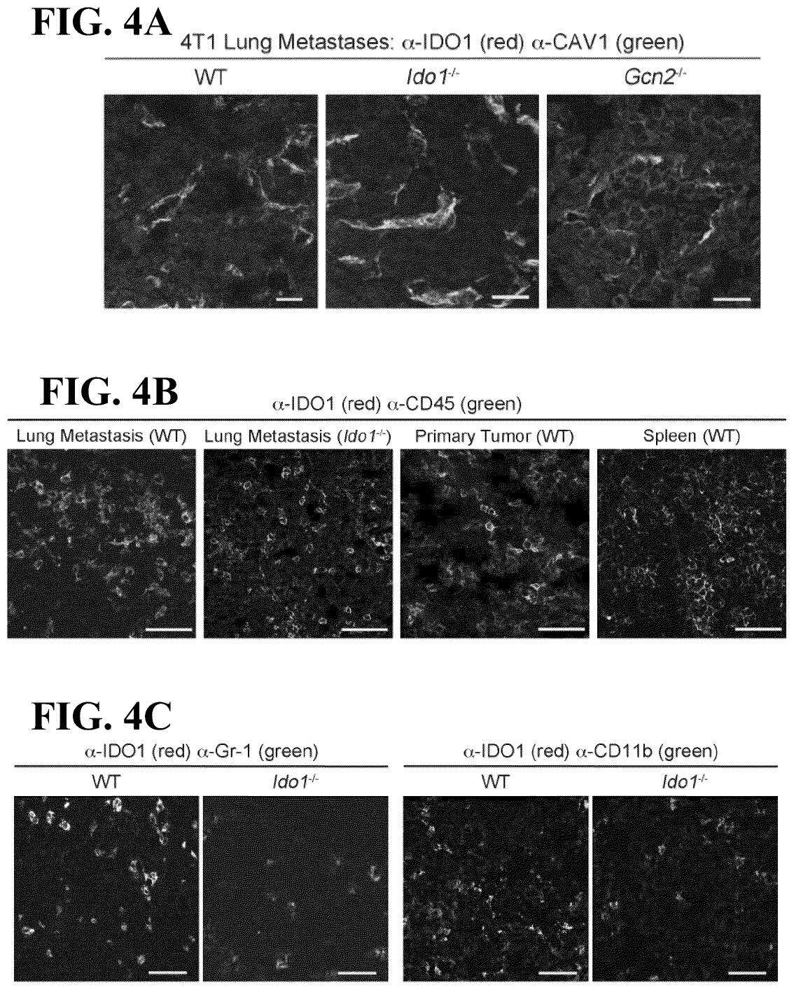

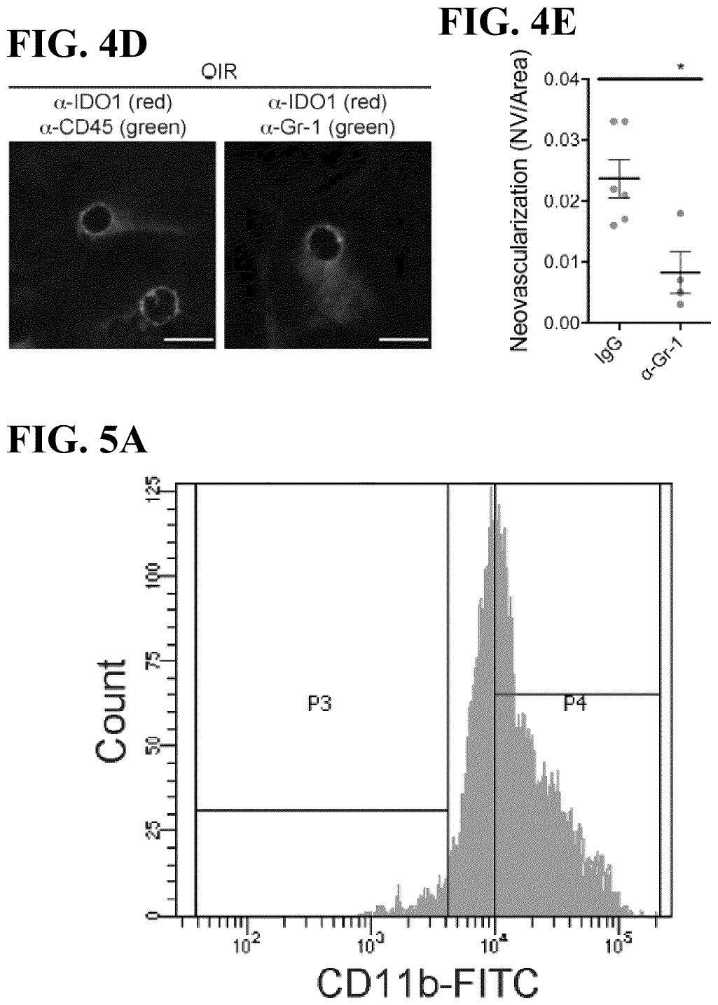

Staining of 4T1 lung metastases for IDO1 revealed that, similar to the OIR retinas, IDO1+ cells were associated with blood vessels in 4T1 metastases established in WT hosts (FIG. 4A). Importantly, no evidence of IDO1 staining was observed in 4T1 metastases established in Ido1^{-/-} hosts (FIG. 4A), confirming both the specificity of the antibody for IDO1 as well as the lack of IDO1 expression by the engrafted 4T1 tumor cells in this setting despite their retention of WT Ido1 alleles. On the other hand, IDO1+ cells were observed in 4T1 metastases established in Gcn2^{-/-} hosts despite the significant reduction in neovascularization observed in these tumors (FIG. 4A), again consistent with GCN2 acting downstream of IDO1.

Although IDO1 positive cells were closely associated with regions of neovascularization in both the OIR and 4T1 lung metastasis models, staining for IDO1 did not appear to directly overlap with staining for the endothelial cell markers used to identify the blood vessels. Instead, the IDO1 staining detected in 4T1 metastatic lesions obtained from WT but not from Ido1^{-/-} hosts overlapped with a subset of cells expressing the common leukocyte marker CD45 (FIG. 4B). In contrast, IDO1 expression was not detected in the CD45+ cells present in primary 4T1 tumors or in the corresponding spleens from WT mice (FIG. 4B). 4T1 tumors elicit a massive expansion of MDSCs (24) predominantly comprised of the granulocytic, polymorphonuclear subset referred as G- or PMN-MDSCs (25), and further analysis of the IDO1-expressing immune cell population in the 4T1 metastases revealed that IDO1 positivity colocalized with a subset of cells expressing Gr-1, a differentiation marker commonly used to identify MDSCs, but not with the other commonly used MDSC marker, CD11b (FIG. 4C). As with 4T1 metastases, the IDO1-expressing cells in retinas from OIR mice were identified as being both CD45+ and Gr-1+ (FIG. 4D). Consistent with these staining data, antibody-mediated depletion of Gr-1+ cells resulted in a significant reduction in OIR-associated neovascularization relative to the isotype antibody-treated control group (FIG. 4E), implicating these as the biologically relevant cells through which IDO1 promotes neovascularization.

### The IDO1-Expressing Subpopulation of Gr-1+ CD11b^{lo} Cells is Functionally Distinct from Conventional MDSCs in its Ability to Promote Neovascularization

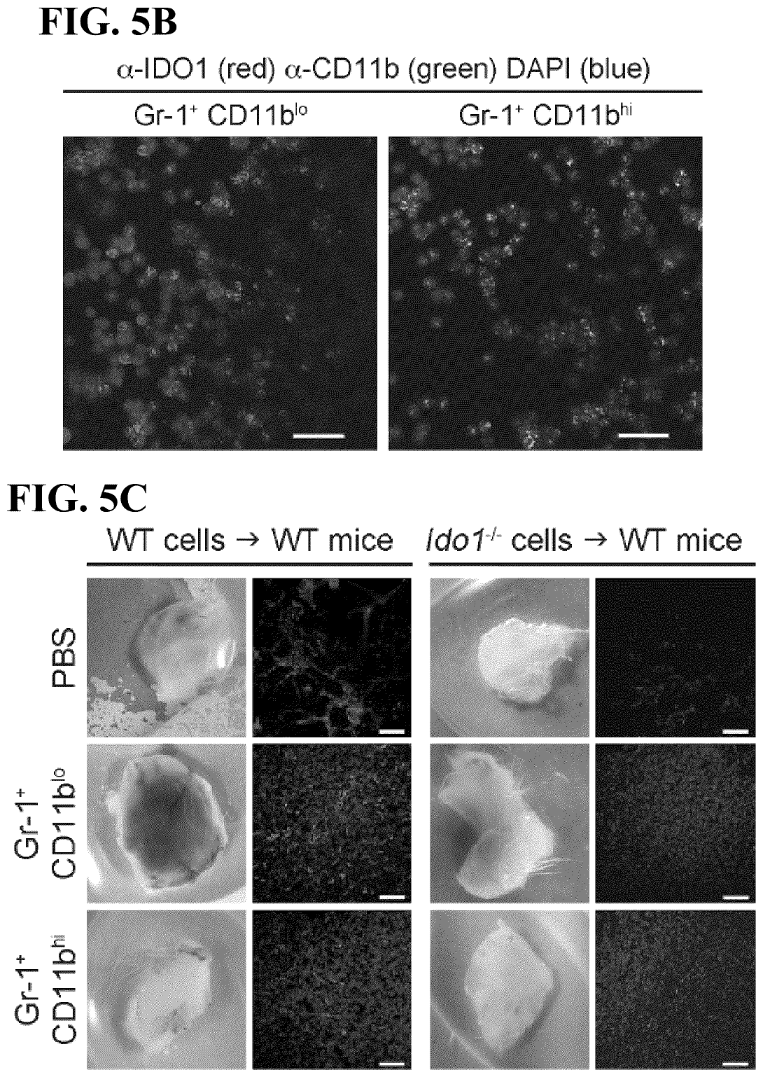

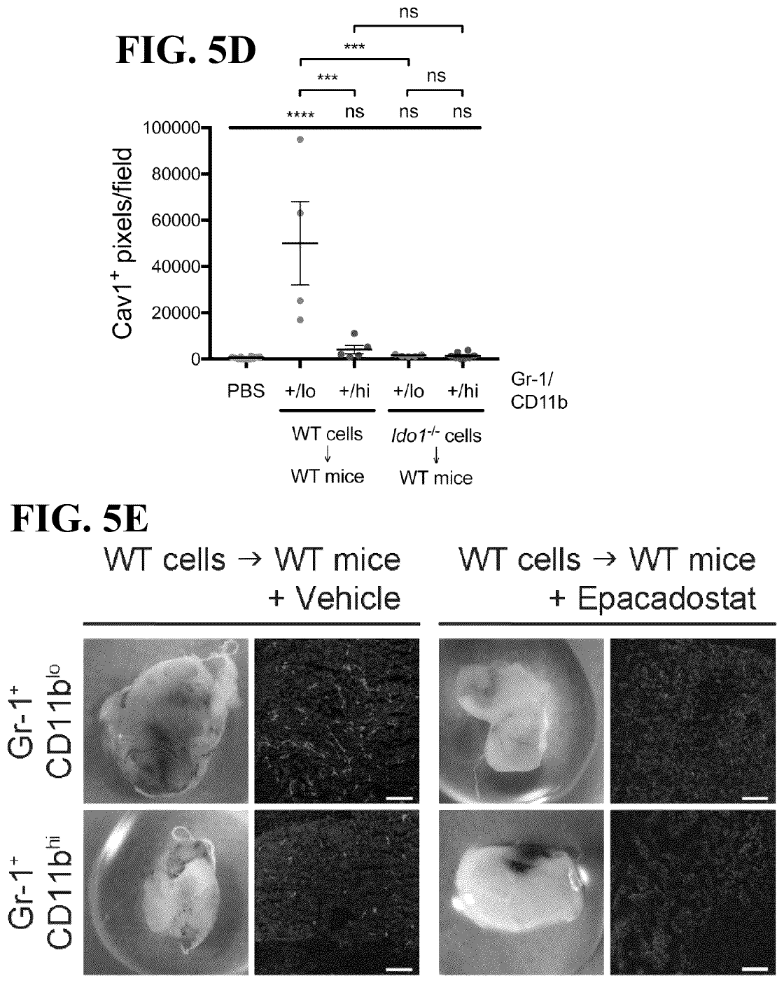

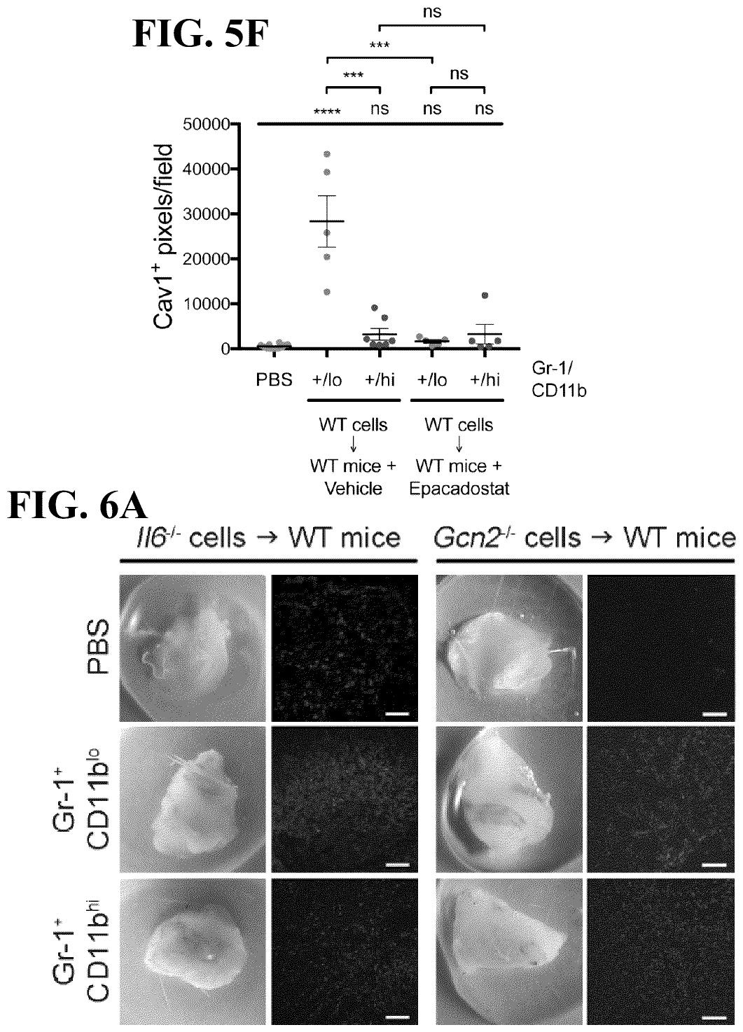

When analyzed by flow cytometry, the vast majority of CD45+ cells isolated from 4T1-metastasis bearing lungs were positive for both Gr-1 and CD11b. Due to the inability of the anti-IDO1 antibody used in these studies to adequately discriminate between IDO1-expressing and non-expressing cells directly by flow cytometry, Gr-1+ cells from dissociated 4T1 lung metastasis were separated based on their level CD11b expression (FIG. 5A and FIG. 12) for microscopy analysis. Immunofluorescence staining demonstrated that within the CD11b^{lo} cell fraction, comprising <5% of the total CD45+ Gr-1+ population, IDO1+ cells were highly enriched, while no evidence of IDO1 expression was detected in the majority CD45+ Gr-1+ CD11b^{hi} population (FIG. 5B). To assess whether the IDO1-expressing subpopulation is functionally capable of promoting neovascularization, sorted Gr-1+ CD11b^{lo} cells isolated from 4T1 lung metastases established in WT hosts were mixed with Matrigel and implanted subcutaneously into naive WT mice. For comparison, Gr-1+ CD11b^{hi} cells, representing conventional MDSCs, were likewise isolated for Matrigel implantation. When the Matrigel plugs were resected 9 days later, it was visually apparent that the plugs incorporating the IDO1-expressing, CD11b^{lo} cells were highly vascularized internally, while the plugs with IDO1-nonexpressing, CD11b^{hi} cells were not (FIG. 5C).

Immunofluorescence analysis of stained sections confirmed a >10-fold differential in mean vascular density between the CD11b^{lo} and CD11b^{hi} plugs (FIG. 5C,D). This result indicates that the CD11b^{lo} population is independently capable of promoting neovascularization, functionally distinguishing these cells from the conventional CD11b^{hi} MDSCs. In contrast, Matrigel plugs incorporated with Ido1^{-/-} CD11b^{lo} cells (isolated from 4T1 metastases established in Ido1^{-/-} mice) showed minimal evidence of blood vessel infiltration (FIG. 5C), which, by immunofluorescence, was quantifiably indistinguishable from plugs incorporated with the corresponding CD11b^{hi} cells and only marginally elevated above the Matrigel plugs not incorporated with any cells (FIG. 5C,D). These results indicate that IDO1 expression is not only associated with, but is critical to the ability of the Gr-1+ CD11b^{lo} cells to promote neovascularization.

### Inhibition of IDO1 Reduces Neovascularization Elicited by Isolated Gr-1+ CD11b^{lo} Cells

To directly test the role of IDO1 enzymatic activity in enabling CD45+ Gr-1+ CD11b^{lo} cells to promote neovascularization, Matrigel plugs incorporated with WT Gr-1+ CD11b^{lo} or Gr-1+ CD11b^{hi} cells were established in WT mice for 6 days after which time the mice were treated with the IDO1 inhibitor epacadostat for 3 days. Evaluation of the resected Matrigel plugs by immunofluorescence staining revealed that epacadostat treatment decreased the degree of neovascularization promoted by CD11b^{lo} cells to a level quantifiably indistinguishable from CD11b^{hi} cells (FIG. 5E,F). Consistent with our prior finding that epacadostat treatment reverses blood vessel formation within established 4T1 metastases (7), these results demonstrate that ongoing IDO1 activity is required to maintain and not just to initiate Gr-1+ CD11lo cell driven neovascularization.

### Neovascularization by Isolated Gr-1+ CD11b^{lo} Cells Requires IL6 and GCN2

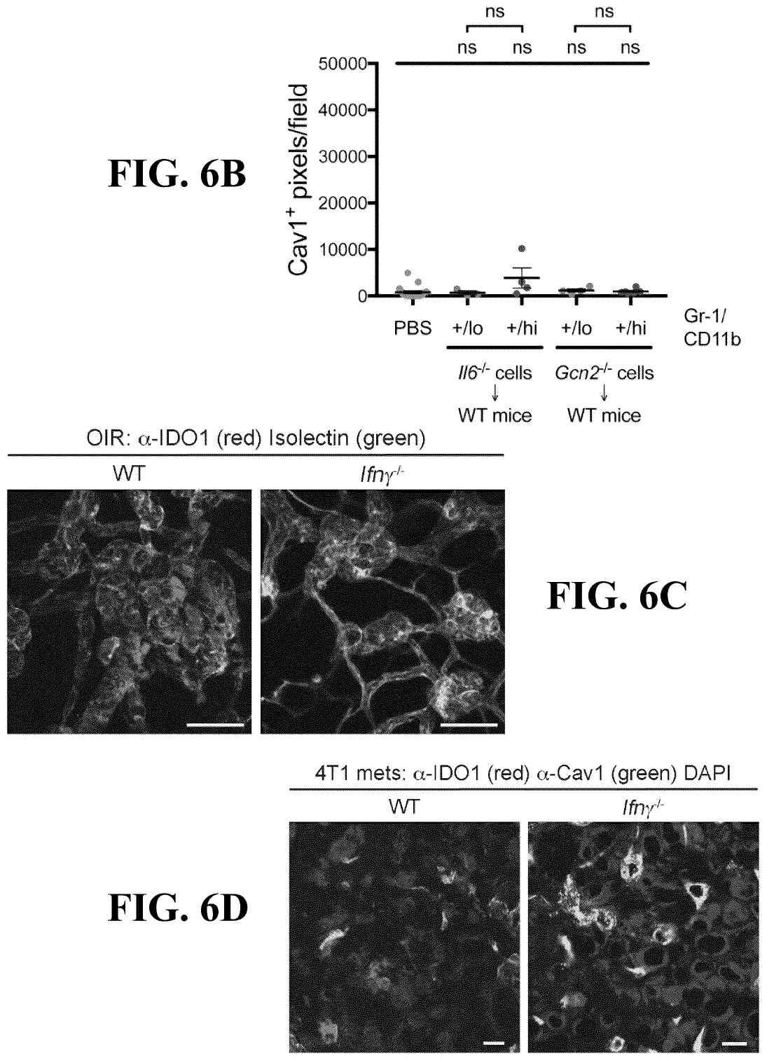

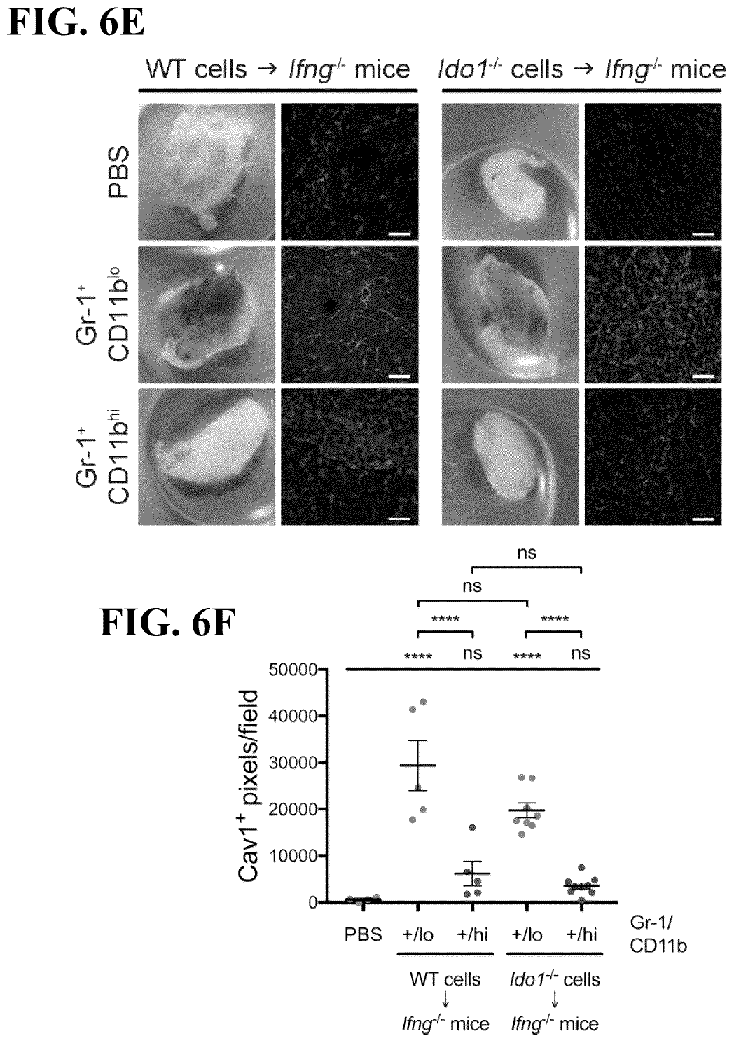

Results from both the 4T1 pulmonary metastasis and OIR models indicated that the ability of IDO1 to promote neovascularization is dependent on the downstream induction of IL6 potentiated through activation of the ISR. To evaluate whether this connection holds true for isolated Gr-1+ CD11^{lo} cells as well, IL6 and GCN2 loss were evaluated genetically. Matrigel plugs incorporated with Gr-1+ CD11b^{lo} cells isolated from either Il6^{-/-} or Gcn2^{-/-} mice showed minimal evidence of blood vessel infiltration (FIG. 6A,B). These results confirm that both IL6 and GCN2 are required for IDO1-expressing, Gr-1+ CD11b^{lo} cells to promote neovascularization, consistent with the intrinsic relevance, in vivo, of this IDO1-driven signaling pathway at the cellular level.

### Elimination of Host IFNγ Restores the Ability of IDO1-Deficient Gr-1+ CD11b^{lo} Cells to Promote Neovascularization

IFNγ is a primary inducer of IDO1 expression but also exerts anti-angiogenic activity. Our previous finding that elimination of IFNγ expression in mice rendered IDO1 inconsequential as a determinant of neovascularization (7) led us to hypothesize that induction of IDO1 may act as a counterregulatory feedback mechanism controlling IFNγ's anti-angiogenic effect. To explicitly test this hypothesis, we carried out studies with mice that genetically lack IFNγ. Evaluation of neovascular regions in OIR retinas and 4T1 metastases revealed a lack of IDO1-positive staining in specimens from Ifng^{-/-} mice (FIG. 6C,D), demonstrating that IFNγ is required for the induction of IDO1 expression in these settings consistent with the hypothesis that IDO1 functions as a downstream counterregulatory element.

To directly evaluate whether IDO1's role in neovascularization is contingent upon the presence of extrinsic IFNγ, isolated Gr-1+ CD11b^{lo} cells from Ido1^{-/-} mice, which were unable to effectively elicit neovascularization when introduced into WT mice, were introduced into Ifng^{-/-} mice. In this context, the introduction of Ido1^{-/-} cells resulted in highly vascularized Matrigel plugs with blood vessel density comparable to plugs incorporated with CD11b^{lo} cells from WT mice (FIG. 6E,F). On the other hand, Gr-1+ CD11b^{hi} cells remained unable to effectively promote neovascularization in Ifng^{-/-} mice irrespective of whether they were derived from WT or Ido1^{-/-} mice (FIG. 6E,F). These data support the conclusion that IDO1 expression in Gr-1+ CD11b^{lo} cells is not determinative of their functional ability to promote neovascularization per se, but rather is critical for protecting the elicited neovasculature from the anti-angiogenic effect of local IFNγ.

### The IDO1+ Vascularizing Subpopulation is Characterized by High Levels of Autofluorescence and Surface Expression of CD11c and asiolo-GM1

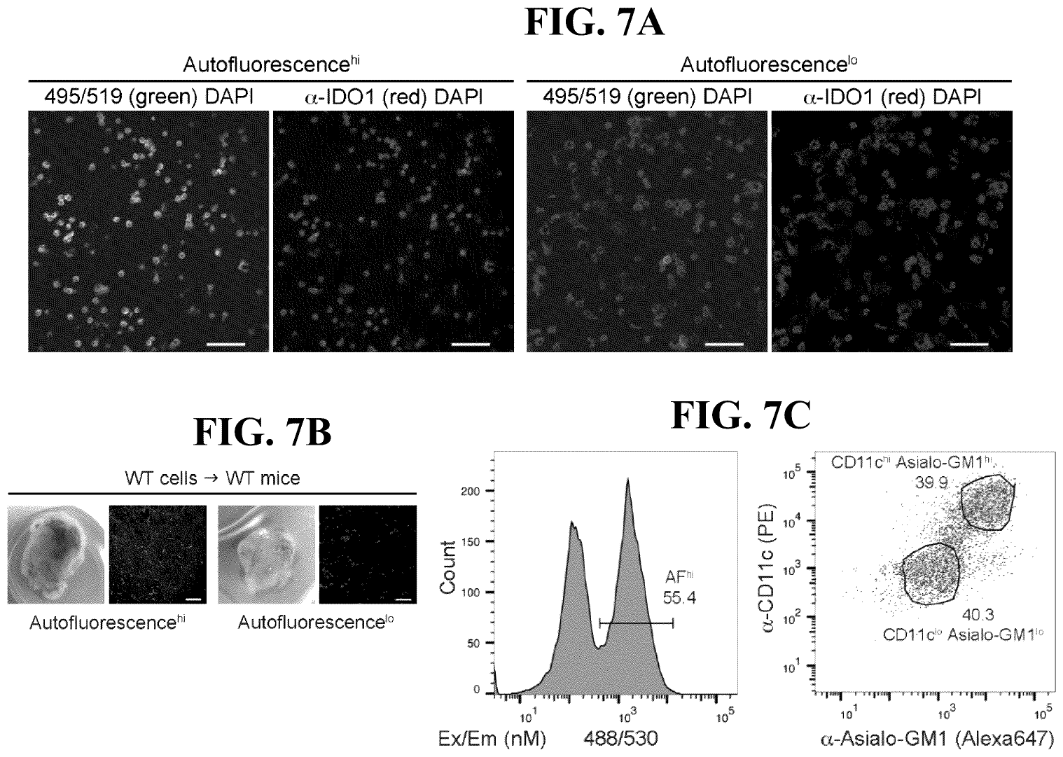

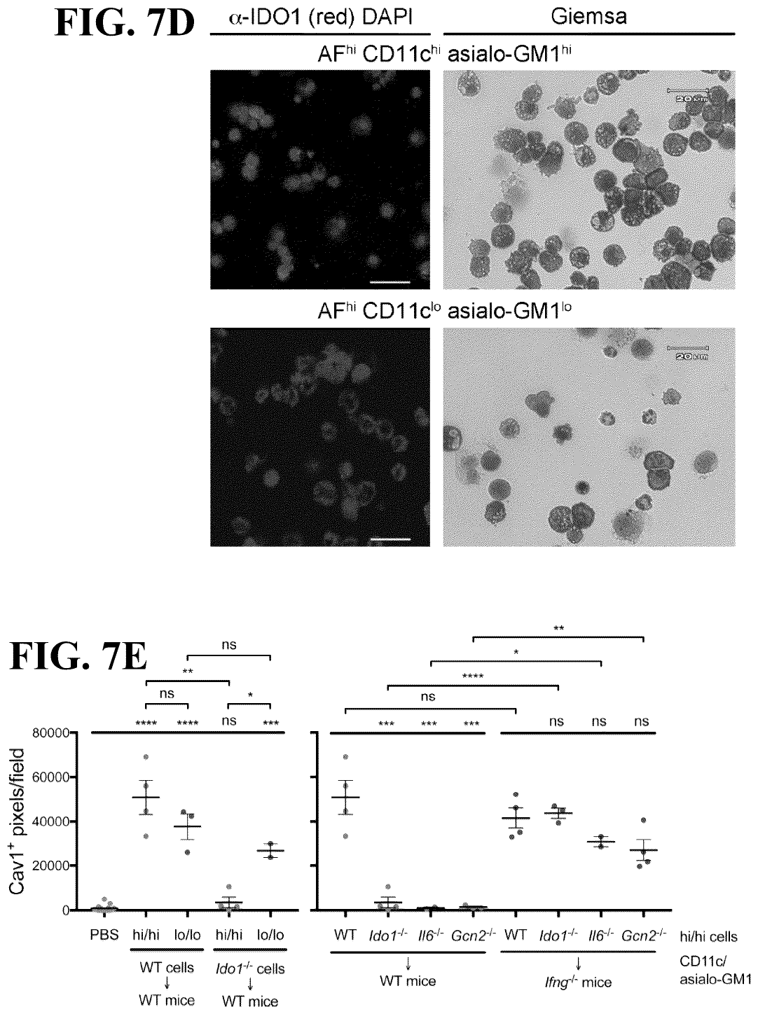

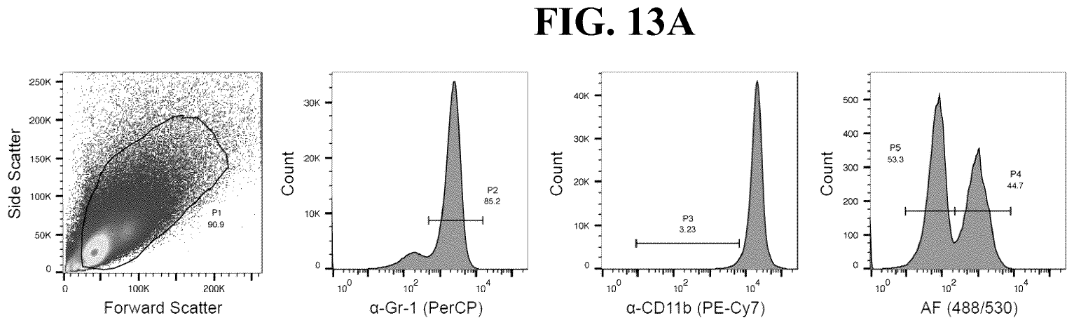

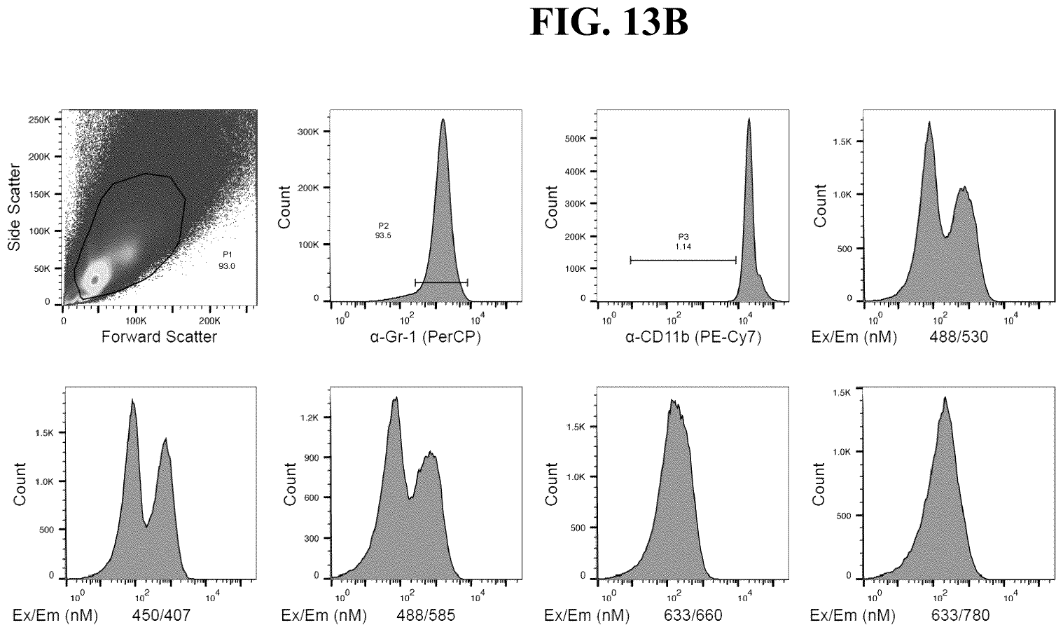

Within the Gr-1+ CD11b^{lo} contingent, further flow cytometry and fluorescence microscopy analysis revealed that IDO1 expression was associated with a subset of cells distinguishable by a high degree of autofluorescence (FIG. 7A and FIG. 13A). When the Gr-1+ CD11b^{lo} population was separated based on high and low autofluorescence (AF^{hi} and AFlo), the ability to promote neovascularization within a Matrigel plug segregated with high autofluorescence signal (FIG. 7B). The autofluorescence signal was particularly strong in three excitation/emission channels 488/530, 450/407 and 488/585 but weaker in the 633/660 and 633/780 channels (FIG. 13B).

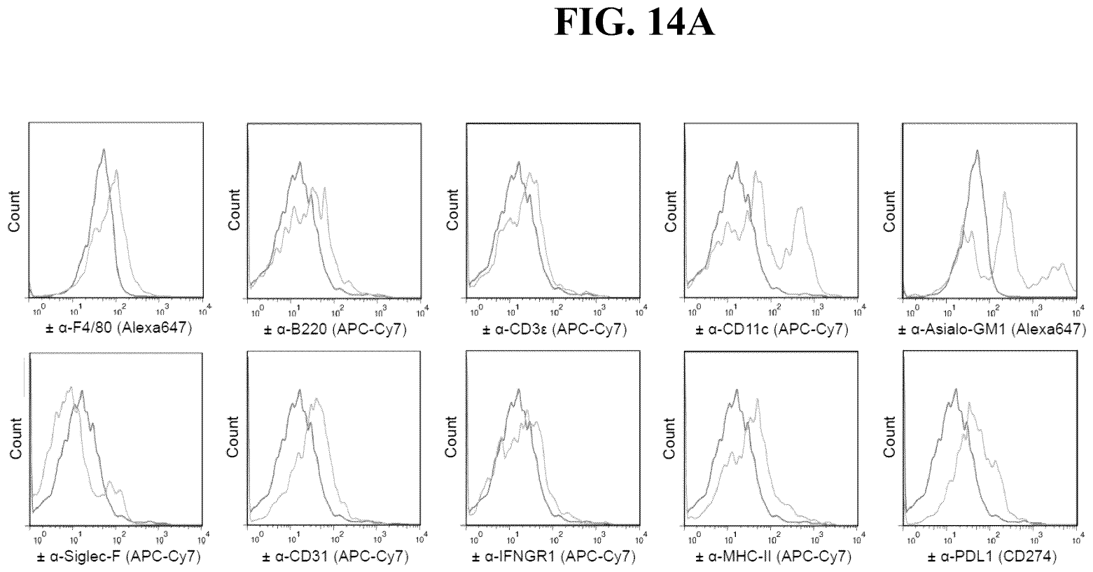

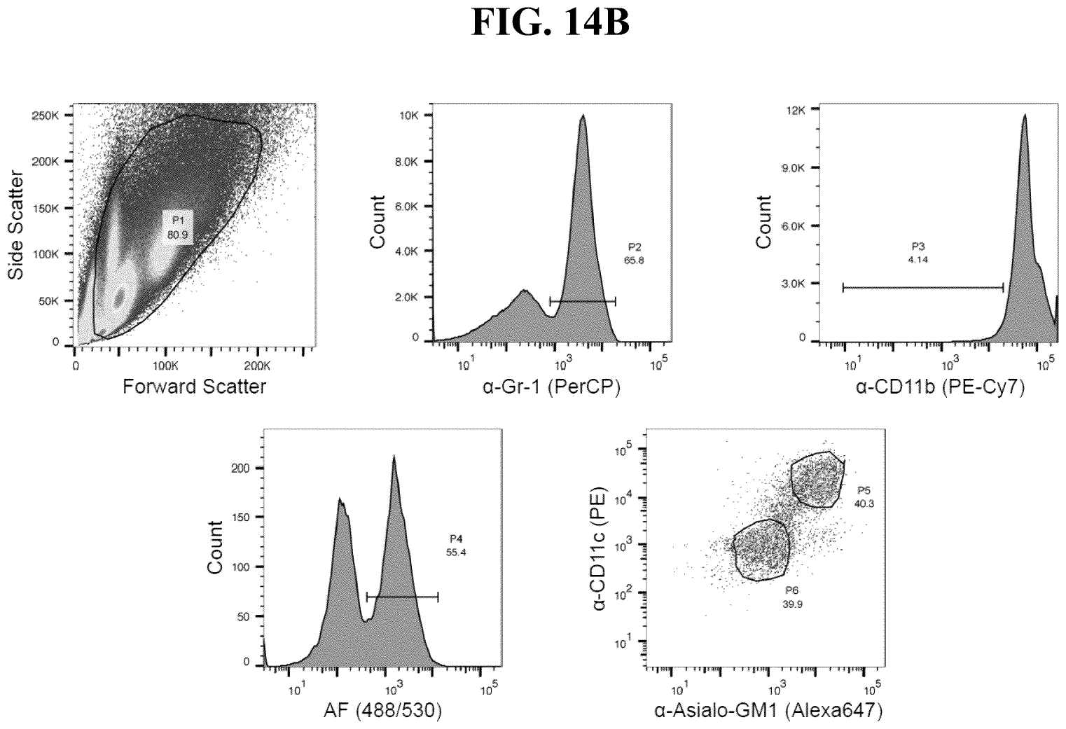

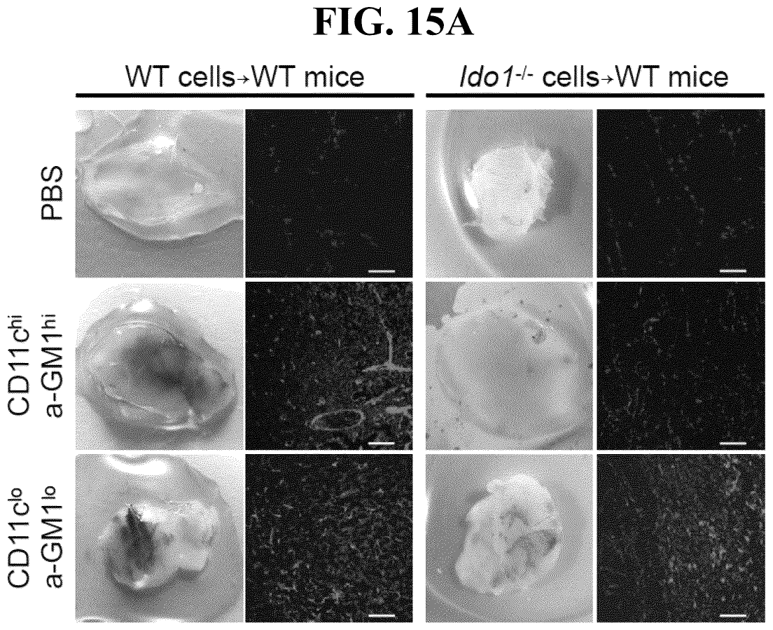

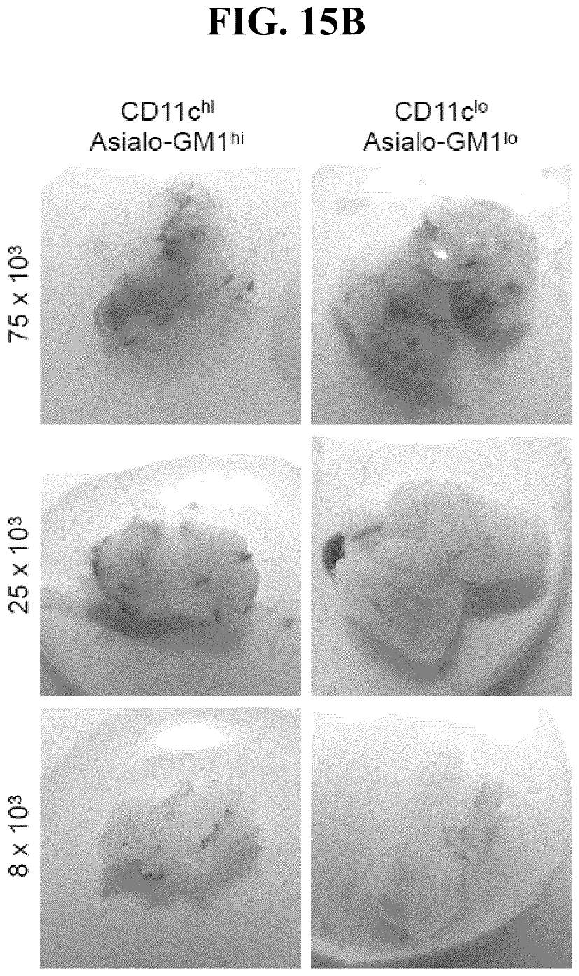

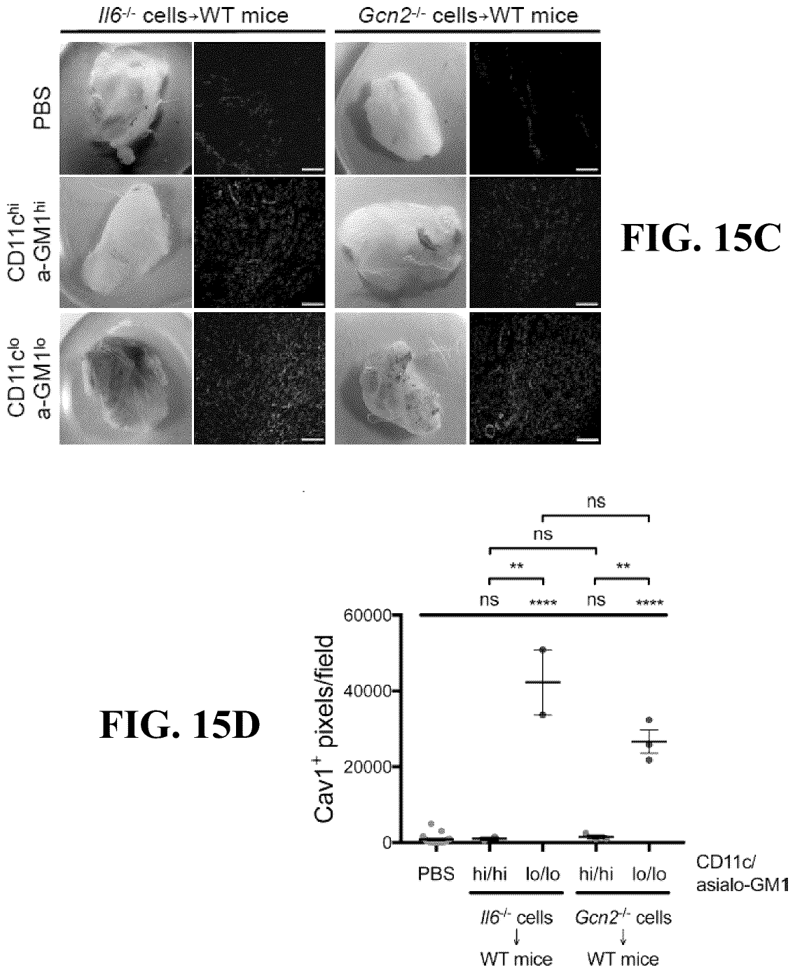

Using the weaker autofluorescence channels, a series of antibodies for detecting different cell surface markers was evaluated on the AF^{hi} cell population. Two antibodies from this panel, directed against CD11c and asialo-GM1, clearly marked discrete subpopulations with strong fluorescence intensity shifts above background (FIG. 14A). Gating on these two markers bifurcated the AF^{hi} population into two predominant groups of CD11c^{hi} asialo-GM1^{hi} and CD11c^{lo} asialo-GM1^{lo} cells (FIG. 7C and FIG. 14B). IDO1 expression segregated almost exclusively with the CD11c^{hi} asialo-GM1^{hi} population which appeared as a morphologically homogeneous population in contrast to the CD11c^{lo} asialo-GM1^{lo} population which was morphologically more heterogeneous (FIG. 7D). Consistent with our previous tissue staining data, no IDO1 expression was observed in CD11c^{hi} asialo-GM1^{hi} cells isolated from Ido1^{-/-} or Ifng^{-/-} mice but was retained in cells from Il6^{-/-} and Gcn2^{-/-} mice (FIG. 14C). Positivity for CD11c and asialo-GM1 as well as for CD45 on the CD11c^{hi} asialo-GM1^{hi} cell population was confirmed not to be attributable to the high background autofluorescence inherent to these cells (FIG. 15), validating the association of these markers with the IDO1-expressing cell population.

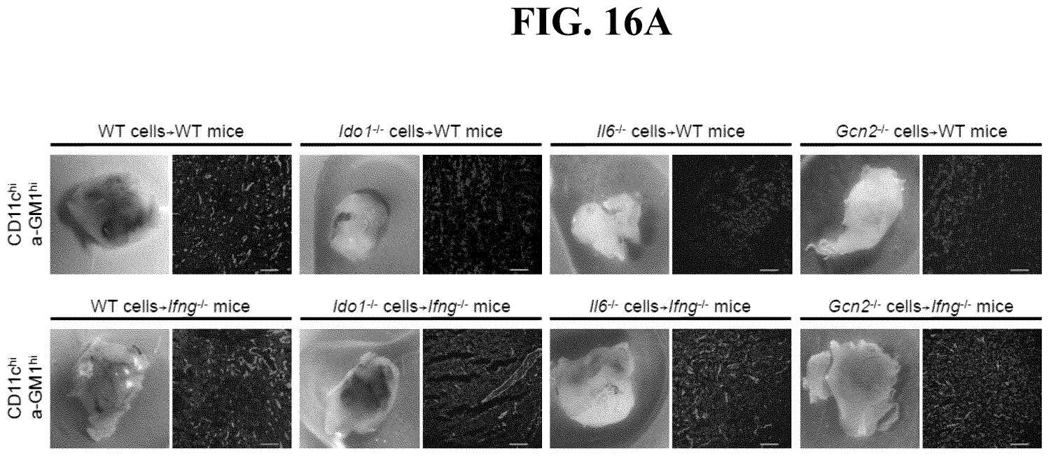

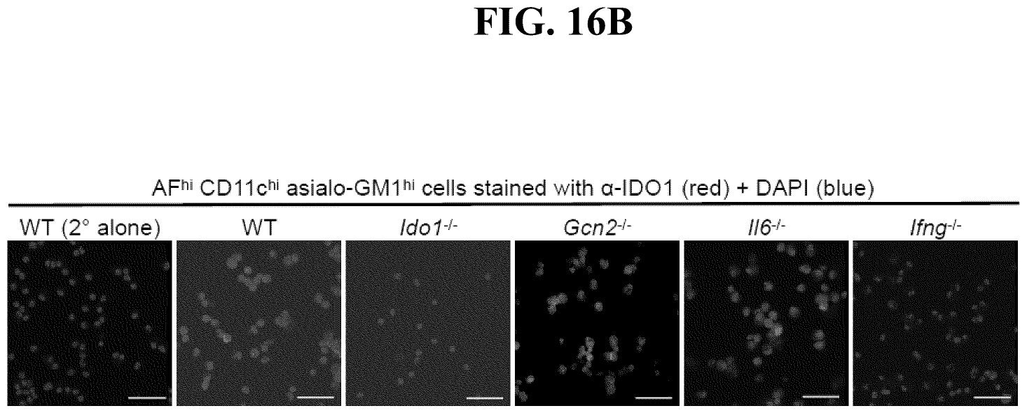

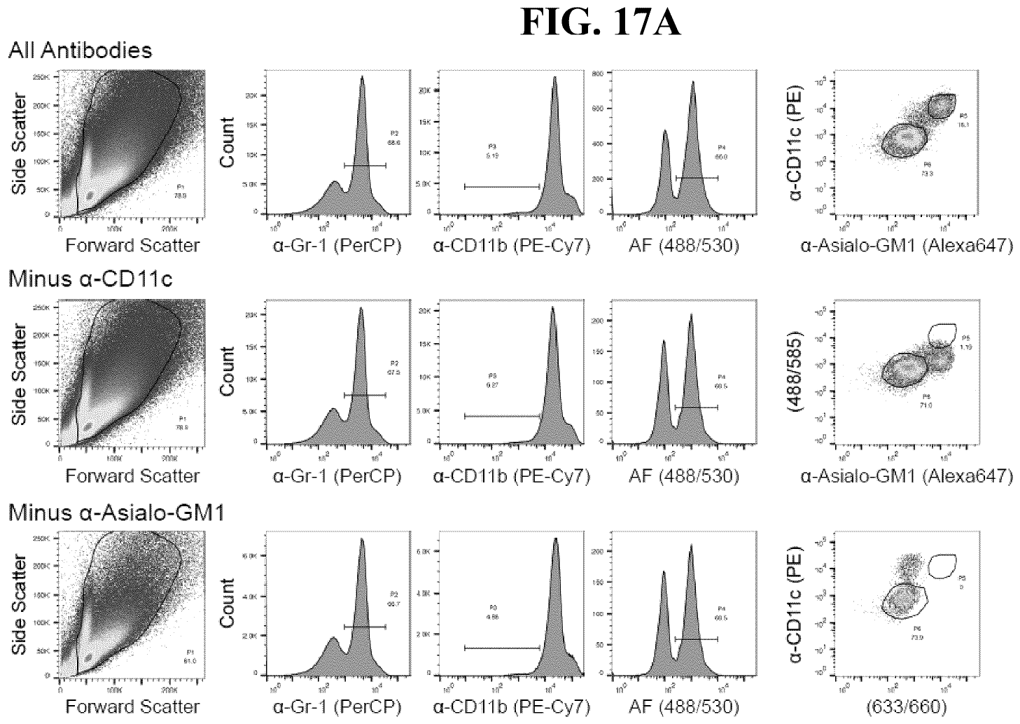

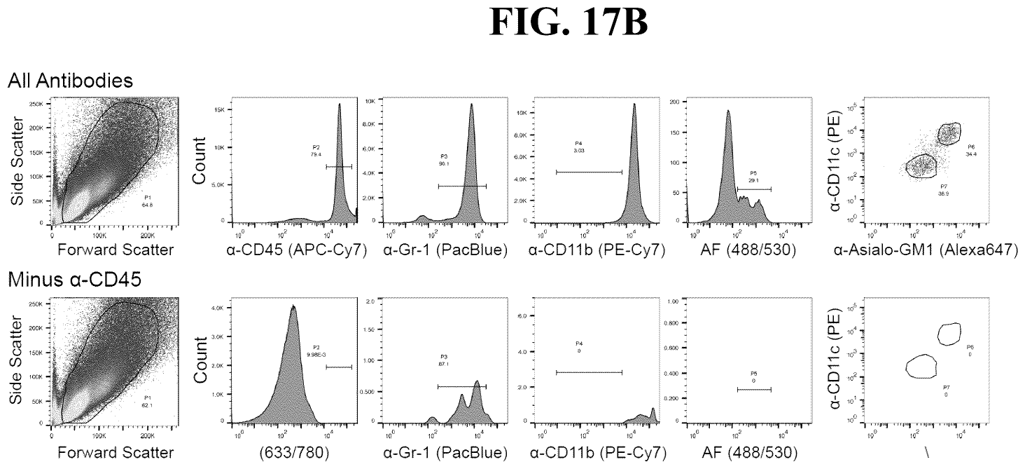

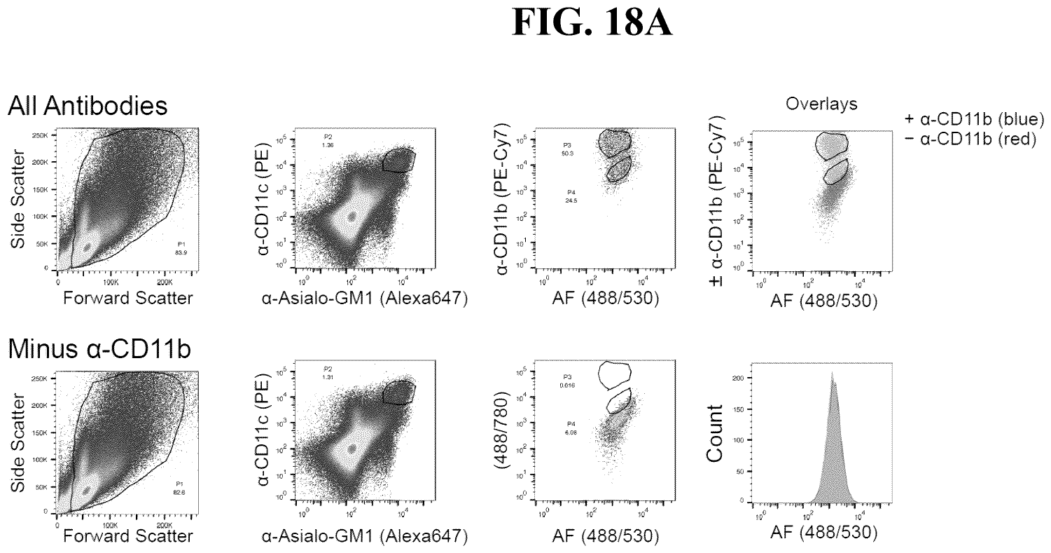

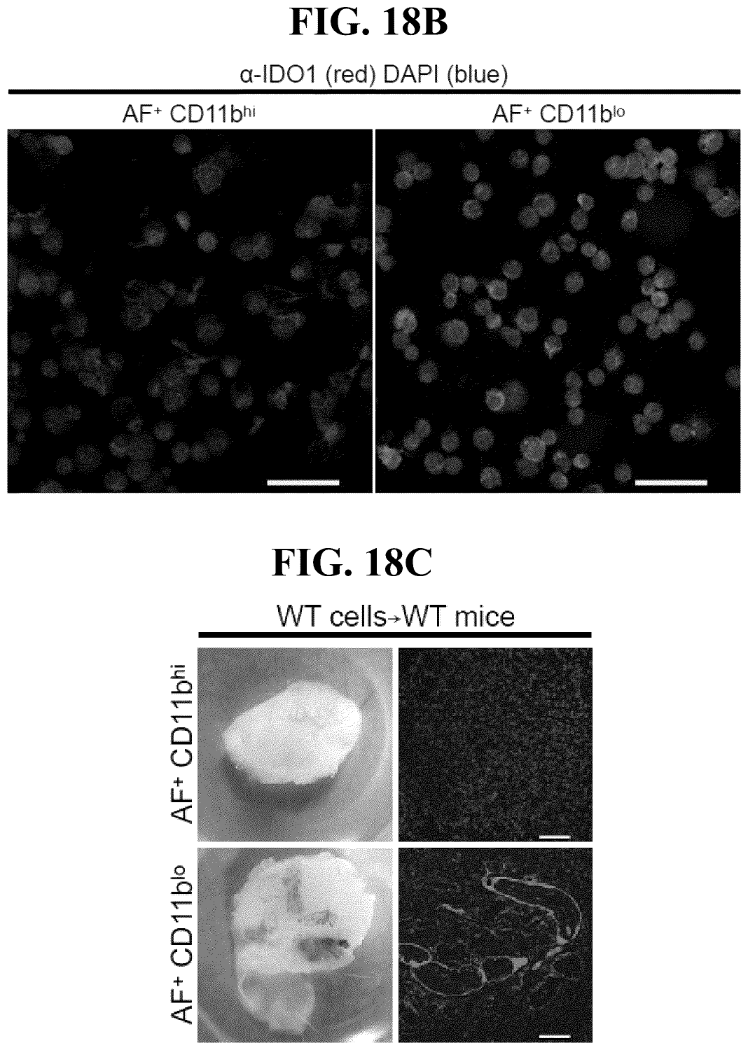

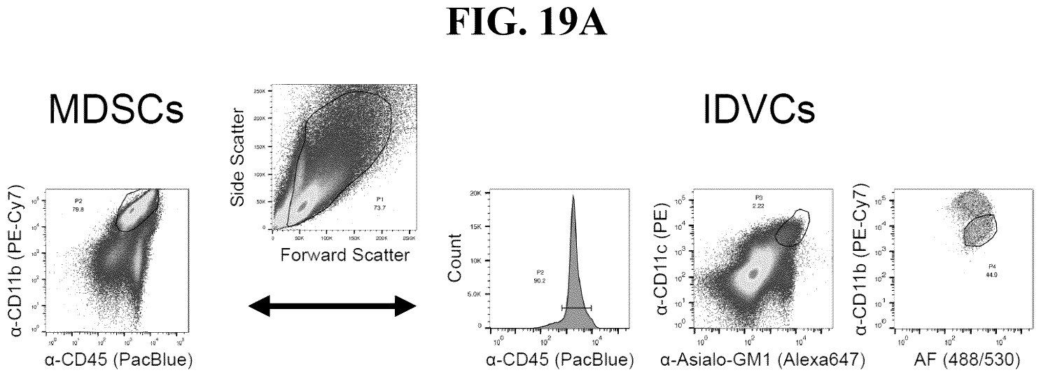

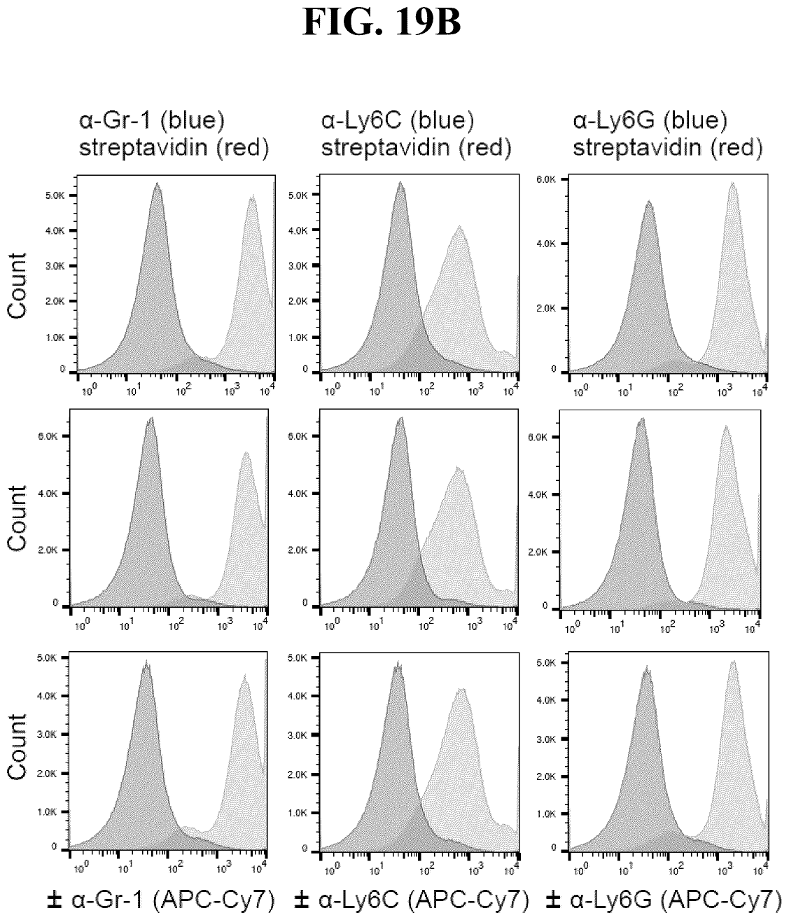

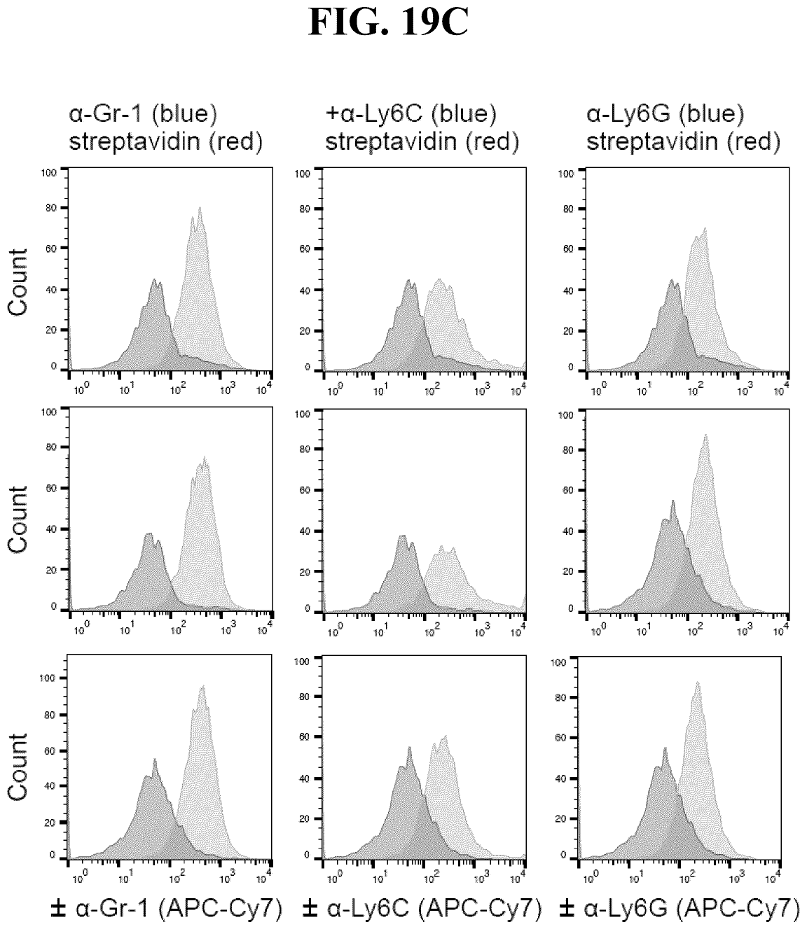

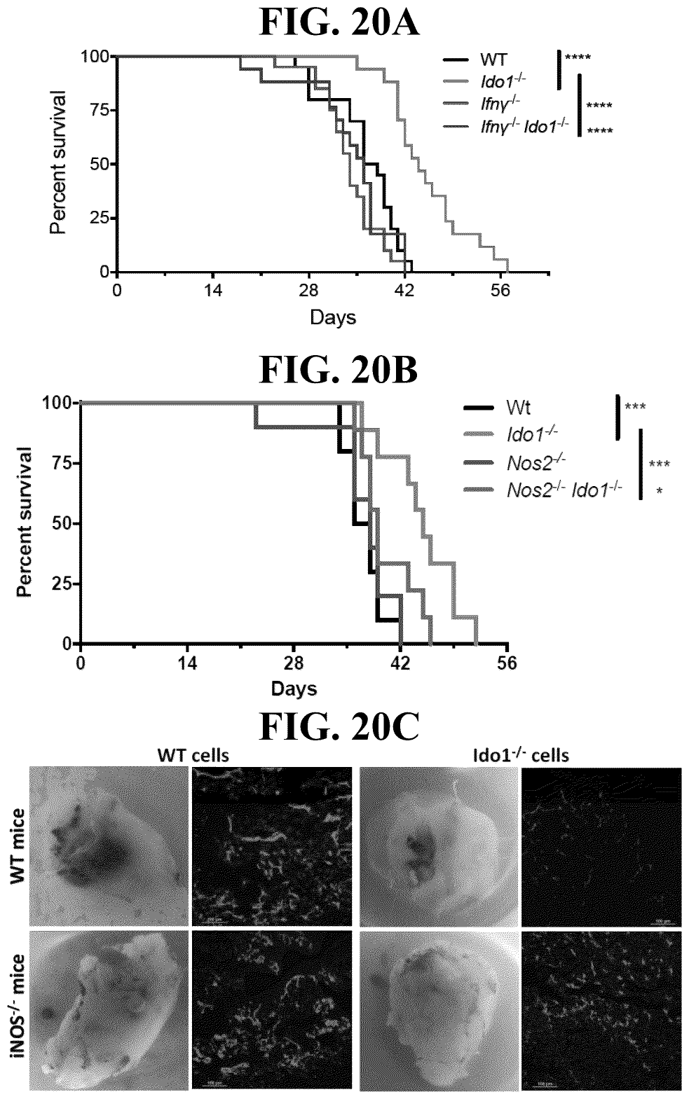

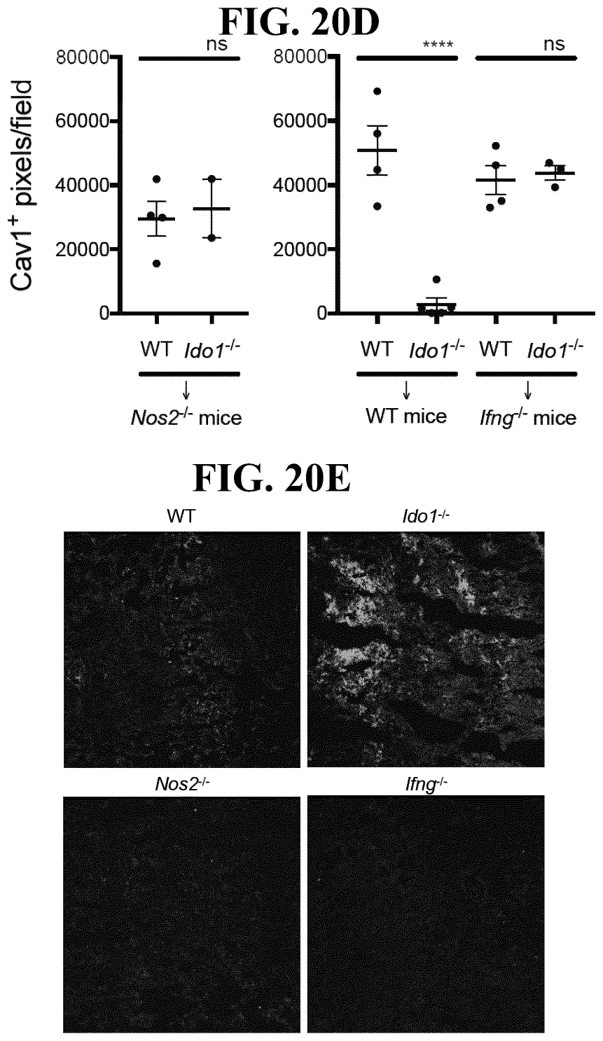

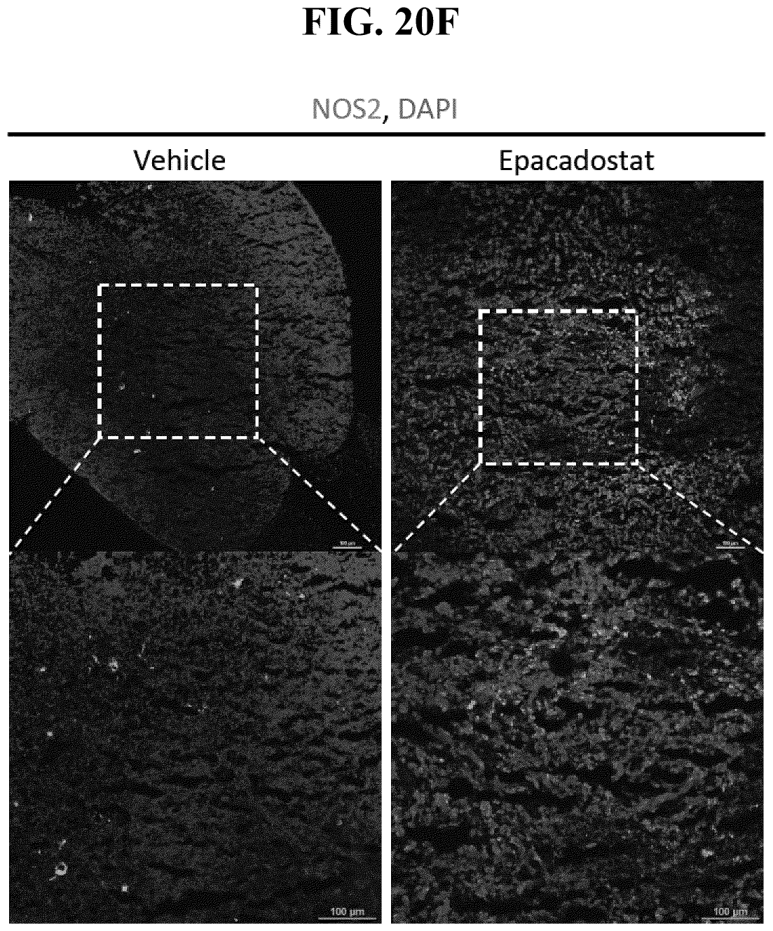

Unexpectedly, when WT cells were transferred to WT recipients, both the CD11c^{hi} asialo-GM1^{hi} (IDO1+) and the CD11c^{lo} asialo-GM1^{lo} (IDO1−) populations were able to promote neovascularization in the Matrigel assay (FIG. 7E and FIG. 16A). Titrating down the number of cells incorporated within the plugs argued against this activity being attributable to the small residual population of IDO1+ cells present within the CD11c^{lo} asialo-GM1^{lo} population, since the corresponding reductions in blood vessel density were too similar between the CD11c^{hi} asialo-GM1^{hi} and CD11clo asialo-GM1^{lo} populations to accommodate this explanation (FIG. 16B). Instead, performing the Matrigel assay with cells from Ido1^{-/-} donors to WT recipients revealed that, when separated in this manner, the CD11c^{lo} asialo-GM1^{lo} population no longer required IDO1 to promote neovascularization while the CD11c^{hi} asialo-GM^{hi} population remained IDO1-dependent (FIG. 7E and FIG. 16A). Likewise, the CD11c^{lo} asialo-GM1^{lo} population lost the requirement for both GCN2 and IL6 while the CD11c^{hi} asialo-GM^{hi} population did not (FIG. 16C,D). These data are consistent with the IDO1+ cells being part of a larger population of AF^{hi} cells that promote neovascularization, but that are distinguishable by their capacity to elicit IFNγ-dependent elimination of these newly formed blood vessels that is, in turn, restrained by their concomitant ability to induce IDO1. The requirement that host IFNγ be present for CD11c^{hi} asialo-GM^{hi} donor cells from Ido1^{-/-}, Il6^{-/-} and Gcn2^{-/-} mice to manifest an impaired capacity to promote neovascularization was confirmed by performing the Matrigel assay with Ifng^{-/-} recipients (FIG. 7E and FIG. 17). Utilizing the CD11c and asialo-GM1 markers for initial identification of the IDVC (IDO1-dependent vascularizing cell) population enabled further characterization of their CD11b and Gr-1 status. CD11b was determined to be low but not absent (FIG. 18), while levels of the composite marker Gr-1 along with its component molecules, Ly6C and Ly6G, were all found to be present at intermediate levels (FIG. 19) further distinguishing the IDVC surface marker profile from conventional MDSCs (Table 5).

TABLE 5
Surface marker comparison between IDVC
and majority PMN-MDSC populations

| IDVCs | PMN-MDSCs |
|---|---|
| CD45⁺ | CD45⁺ |
| Gr-1^{int} | Gr-1^{hi} |
| CD11b^{lo} | CD11b^{hi} |
| Ly6G^{int} | Ly6G^{hi} |
| Ly6C^{int} | Ly6C^{int} |
| AF^{hi} | AF^{lo} |
| CD11c^{hi} | CD11c⁻ |
| Asialo-GM1^{hi} | Asialo-GM1⁻ |

### EXAMPLE 3: DISCUSSION

In contrast to the detailed insights that have come from studies into the ability of IDO1 to dampen T cell responses (26), the case for IDO1's role as an integral mediator of inflammatory neovascularization has thus far been highly conceptual (7). Here we elucidate the molecular and cellular underpinnings of this heretofore unrecognized aspect of IDO1 biology. Detection of the IDO1-expressing cells associated with regions of neovascularization by immunofluorescence microscopy led to the identification of a unique subset of immune cells present amidst but distinguishable from conventional MDSCs that, through induction of IDO1, are capable of opposing the anti-angiogenic activity of IFNγ and promoting the in vivo formation of new blood vessels. Examination of how IDO1 induction in these cells counterbalances IFNγ has delineated the downstream involvement of signaling initiated by GCN2 and propagated through the ISR pathway leading to the induction of IL6 that is needed to promote neovascularization. In toto, these findings reveal fundamental new insights into this novel facet of IDO1 biology. By initiating the breakdown of tryptophan, IDO1 can potentially signal through both the depletion of tryptophan and production of catabolites, and there has been ongoing debate over which of these two possible signaling mechanisms is most relevant (27,28). Biological evidence has been reported for signaling through AHR in response to the catabolic product kynurenine and signaling through GCN2 in response to tryptophan depletion (15,29). With regard to IDO1's impact on neovascularization, our data fall clearly on the side of tryptophan depletion. Targeting key components of the ISR in vivo, including GCN2, CHOP and ATF4, all phenocopy the effects that loss of either IDO1 or IL6 has on neovascularization. Importantly, even though disrupting key components of the ISR was phenotypically comparable to eliminating IDO1, IDO1 expression and activity remained elevated. In the debate over mechanisms of immune regulation, one of the principle objections against the tryptophan depletion hypothesis has been that the systemic level of tryptophan is too high for IDO1-expressing DCs to extrinsically influence T cell functionality through a depletion-based mechanism of action (30). However, in the case of IDO1's influence on neovascularization this concern is rendered moot by evidence, both in vitro and in vivo, that IFNγ-driven induction of IDO1-mediated tryptophan catabolism acts intrinsically within cells to initiate ISR pathway activation and downstream IL6 induction. Given the massive expansion of MDSCs that occurs in mice harboring 4T1 tumors, our determination that Gr-1, a lineage marker for MDSCs and neutrophils, overlapped with IDO1 expression in 4T1 metastases led to the initial supposition that these IDO1-expressing cells might be conventional MDSCs. However, the lack of overlap between IDO1 expression and CD11b, the other characteristic lineage marker of both PMN- and M-MDSCs, differentiated the IDO1+ cells, (referred to for this study as IDO1-dependent vascularizing cells or IDVCs), from the bulk of MDSCs as currently defined (31). A high degree of inherent autofluorescence was subsequently identified as a defining physical characteristic associated with the IDO1+ cell population, which enabled further refinement of the IDO1+ cells but also presented an additional complication for flow cytometry-based characterization. Utilizing channels with the lowest autofluorescence signal, the IDO1-expressing cells were further delineated to near homogeneity with the two cell surface markers, CD11c and asialo-GM1, typically used to identify dendritic cells and natural killer cells respectively. While both AF^{hi} cell populations could elicit neovascularization in the Matrigel assay, only the CD11c^{hi} asialo-GM1^{hi} subset required IDO1 or the downstream components GCN2 and IL6. The observation that isolated CD11c^{lo} asialo-GM1^{lo} cells no longer required IDO1 to effectively promote Matrigel neovascularization while the mixed population of CD11c^{lo} asialo-GM1^{lo} and CD11c^{hi} asialo-GM1^{hi} cells did implies that, in this context, the IDO1-expressing CD11c^{hi} asialo-GM1^{hi} population is responsible not only for counteracting the IFNγ-mediated anti-angiogenic response but also for provoking it.

The lack of detectable IDO1 expression in the broader population of conventional Gr-1+ CD11b^{hi} MDSCs is consistent with our previous findings (9), however, published evidence of IDO1 expression directly in MDSCs has also been reported. In the case of murine MDSCs, the evidence has generally consisted of expression analysis within populations of cells by PCR or Western blot analysis (32-34). By not examining IDO1 expression at the individual cell level, such studies carry the risk of being misled by contamination of the conventional MDSC population with the IDVCs identified here. There is also the concern that many of the commercially available antibodies for detecting IDO1 expression lack specificity that may lead to spurious positive results (35), an issue that was addressed in the current study by the development of an antibody that specifically recognizes IDO1 in mouse tissues using the germline knockout strain for validation (36). Thus, the widespread notion that elevated expression of IDO1 occurs in conventionally-defined, murine MDSCs should be viewed with skepticism until more definitive evidence can be provided.

Functionally, the ability to promote angiogenesis is currently one of the defining characteristics of conventional MDSCs (4), however, our data suggest that a reassessment of this attribution may also be in order. While the minor subpopulation of AF^{hi} cells identified in this study was highly effective at promoting neovascularization within an engrafted Matrigel plug, conventional MDSCs, were, by comparison, quite ineffectual. This raises the possibility that the proangiogenic activity generally ascribed to conventional MDSCs may instead be limited to this minor subset of AF^{hi} cells. Studies conducted using the Matrigel plug assay, for instance, have ostensibly associated isolated MDSCs with angiogenic activity. However, in instances where MDSCs were isolated based solely on their Gr-1+ (or Ly6C/Ly6G) status (37), there is clearly the likelihood of inadvertent contamination with Gr-1+ CD11b^{lo} AF^{hi} cells. Even when MDSCs were isolated based on their combined Gr-1+ CD11b+ status (38), crossover contamination with CD11b^{lo} AF^{hi} cells could still be an issue due to insufficient stringency of the CD11b cutoff or inadvertent gating on non-specific autofluorescence. Based on these findings, a careful reassessment of the angiogenic activity of conventional MDSCs is clearly in order. From these studies it is apparent that IDO1 is not simply a marker that distinguishes IDVCs from MDSCs, but rather is functionally required for these cells to promote neovascularization. IDO1 is not, however, an angiogenic factor that directly promotes the formation of new blood vessels like VEGF. Rather, our results demonstrate that IDO1's primary role in IDVCs is to counteract the anti-neovascular effect of IFNγ. Mechanistically, how IFNγ exerts its anti-angiogenic effect remains to be fully understood. Recent studies have demonstrated that IFNγ acts directly on endothelial cells, as targeted deletion of the IFNGR in endothelial cells, but not in immune cells, was sufficient to protect the vasculature (39) and downregulation of Dll4 (delta-like ligand 4)/Notch signaling in endothelial cells has been implicated in this process (40). These data suggest a potential key for elucidating the mechanism may lie in the observed ability of IL6 produced by IDVCs to counteract IFNγ. While IL6 has been demonstrated to stimulate new blood vessel formation through a VEGF-independent mechanism (41), our data suggest that the production of IL6 by IDVCs is more relevant as a check on the anti-neovascular activity of IFNγ than as a direct promoter of angiogenesis. Additionally, while the current study has focused on neovascularization, IL6 is a pleiotropic cytokine that has also been implicated in eliciting functionally suppressive MDSCs (42). Our previous studies have shown that MDSCs garnered from Ido1^{-/-} mice bearing 4T1 tumors exhibited diminished suppressive activity which could be restored by implantation of modified tumor cells that ectopically expressed IL6 (9). Future studies will be required to investigate whether the IDO1-dependent production of IL6 by IDVCs may, in conjunction with promoting neovascularization, also contribute to MDSCs developing their full suppressive potential.

While the genetic studies demonstrate the integral role of IDO1 in inflammatory neovascularization, the additional studies with epacadostat extend this concept even further by indicating that IDO1 inhibitors are capable of reversing the neovascularization process once initiated, consistent with an ongoing role for IDO1 in maintaining blood vessel integrity. One of the challenges of anti-angiogenic therapy with VEGF-targeting agents is the development of acquired resistance. In this regard, both the recruitment of MDSCs and IL6 signaling have been implicated in the failure of VEGF-directed antibody therapies in patients (43,44). These observations are consistent with the existence of a human equivalent to the murine IDVCs identified in this study that contributes to the acquired resistance to VEGF antibody therapy.

Each and every patent, patent application, and publication, including websites and other publications cited throughout the specification, is incorporated herein by reference. While the invention has been described with reference to particular embodiments, it will be appreciated that modifications can be made without departing from the spirit of the invention. Such modifications are intended to fall within the scope of the appended claims.

### REFERENCES

1. Folkman J. Angiogenesis: an organizing principle for drug discovery? Nat Rev Drug Discov 2007;6(4):273-86.
2. Dvorak H F. Tumors: wounds that do not heal-redux. Cancer Immunol Res 2015;3(1):1-11.
3. Gabrilovich D I. Myeloid-Derived Suppressor Cells. Cancer Immunol Res 2017;5(1):3-8.
4. Johnson B W, Achyut B R, Fulzele S, Mondal A K, Kolhe R, Arbab A S. Delineating Pro-Angiogenic Myeloid Cells in Cancer Therapy. Int J Mol Sci 2018;19(9).
5. Nilsson M B, Langley R R, Fidler I J. Interleukin-6, secreted by human ovarian carcinoma cells, is a potent proangiogenic cytokine. Cancer Res 2005;65(23):10794-800.
6. Qin Z, Blankenstein T. CD4+ T cell-mediated tumor rejection involves inhibition of angiogenesis that is dependent on IFN gamma receptor expression by nonhematopoietic cells. Immunity 2000;12(6):677-86.
7. Mondal A, Smith C, DuHadaway J B, Sutanto-Ward E, Prendergast G C, Bravo-Nuevo A, et al. IDO1 is an Integral Mediator of Inflammatory Neovascularization. EBioMedicine 2016;14:74-82.
8. Taylor M W, Feng G S. Relationship between interferon-gamma, indoleamine 2,3-dioxygenase, and tryptophan catabolism. FASEB J 1991;5(11):2516-22.
9. Smith C, Chang M Y, Parker K H, Beury D W, Duhadaway J B, Flick H E, et al. IDO Is a nodal pathogenic driver of lung cancer and metastasis development. Cancer Discov 2012;2(8):722-35.
10. Metz R, Smith C, DuHadaway J B, Chandler P, Baban B, Merlo L M, et al. IDO2 is critical for IDO1-mediated T-cell regulation and exerts a non-redundant function in inflammation. Int Immunol 2014;26(7):357-67.
11. Liu H, Huang L, Bradley J, Liu K, Bardhan K, Ron D, et al. GCN2-dependent metabolic stress is essential for endotoxemic cytokine induction and pathology. Mol Cell Biol 2014;34(3):428-38.
12. Sharma M D, Hou D Y, Liu Y, Koni P A, Metz R, Chandler P, et al. Indoleamine 2,3-dioxygenase controls conversion of Foxp3+ Tregs to TH17-like cells in tumor-draining lymph nodes. Blood 2009;113(24):6102-11.
13. Litzenburger U M, Opitz C A, Sahm F, Rauschenbach K J, Trump S, Winter M, et al. Constitutive IDO expression in human cancer is sustained by an autocrine signaling loop involving IL-6, STAT3 and the AHR. Oncotarget 2014;5(4):1038-51.
14. Prendergast G C, Malachowski W P, DuHadaway J B, Muller A J. Discovery of IDO1 Inhibitors: From Bench to Bedside. Cancer Res 2017;77(24):6795-811.
15. Munn D H, Sharma M D, Baban B, Harding H P, Zhang Y, Ron D, et al. GCN2 Kinase in T Cells Mediates Proliferative Arrest and Anergy Induction in Response to Indoleamine 2,3- Dioxygenase. Immunity 2005;22(5):633-42.
16. Dey S, Sutanto-Ward E, Kopp K L, DuHadaway J, Mondal A, Ghaban D, et al. Peptide vaccination directed against IDO1-expressing immune cells elicits CD8+ and CD4+ T-cellmediated antitumor immunity and enhanced anti-PD1 responses. J Immunother Cancer 2020;8(2):e000605.
17. Connor K M, Krah N M, Dennison R J, Aderman C M, Chen J, Guerin K I, et al. Quantification of oxygen-induced retinopathy in the mouse: a model of vessel loss, vessel regrowth and pathological angiogenesis. Nat Protoc 2009;4(11):1565-73.
18. Muller A J, DuHadaway J B, Jailer D, Curtis P, Metz R, Prendergast G C. Immunotherapeutic suppression of indoleamine 2,3-dioxygenase and tumor growth with ethyl pyruvate. Cancer Res 2010;70(5):1845-53.
19. Thevenot P T, Sierra R A, Raber P L, Al-Khami A A, Trillo-Tinoco J, Zarreii P, et al. The stress-response sensor CHOP regulates the function and accumulation of myeloid-derived suppressor cells in tumors. Immunity 2014;41(3):389-401.
20. Ron D. Translational control in the endoplasmic reticulum stress response. J Clin Invest 2002;110(10):1383-88.
21. Matsushita N, Sogawa K, Ema M, Yoshida A, Fujii-Kuriyama Y. A factor binding to the xenobiotic responsive element (XRE) of P-4501A1 gene consists of at least two helix-loophelix proteins, Ah receptor and Arnt. J Biol Chem 1993;268(28):21002-06.
22. Sidrauski C, Acosta-Alvear D, Khoutorsky A, Vedantham P, Hearn B R, Li H, et al. Pharmacological brake-release of mRNA translation enhances cognitive memory. Elife 2013;2:e00498.
23. Schmees N, Gutcher I, Roehn U, Irlbacher H, Stoeckigt D, Bader B, et al. Identification of BAY-218, a potent and selective small-molecule AhR inhibitor, as a new modality to counteract tumor immunosuppression [abstract]. In: Proceedings of the American Association for Cancer Research Annual Meeting 2019; 2019 Mar 29-Apr 3; Atlanta, GA. Philadelphia (PA): AACR; Cancer Res 2019;79(13 Suppl): Abstract nr 4454.
24. Sinha P, Clements V K, Ostrand-Rosenberg S. Reduction of myeloid-derived suppressor cells and induction of M1 macrophages facilitate the rejection of established metastatic disease. J Immunol 2005;174(2):636-45.
25. Bosiljcic M, Cederberg R A, Hamilton M J, LePard N E, Harbourne B T, Collier J L, et al. Targeting myeloid-derived suppressor cells in combination with primary mammary tumor resection reduces metastatic growth in the lungs. Breast Cancer Res 2019;21(1): 103.
26. Munn D H, Mellor A L. Indoleamine 2,3 dioxygenase and metabolic control of immune responses. Trends Immunol 2013;34(3):137-43.
27. Mellor A L, Munn D H. Tryptophan catabolism and T-cell tolerance: immunosuppression by starvation? Immunol Today 1999;20:469-73.
28. Moffett J R, Namboodiri M A. Tryptophan and the immune response. Immunol Cell Biol 2003;81(4):247-65.
29. Opitz C A, Litzenburger U M, Sahm F, Ott M, Tritschler I, Trump S, et al. An endogenous tumour-promoting ligand of the human aryl hydrocarbon receptor. Nature 2011;478(7368):197-203.
30. Badawy A A, Namboodiri A M, Moffett J R. The end of the road for the tryptophan depletion concept in pregnancy and infection. Clin Sci (Lond) 2016;130(15):1327-33.
31. Bronte V, Brandau S, Chen S H, Colombo M P, Frey A B, Greten T F, et al. Recommendations for myeloid-derived suppressor cell nomenclature and characterization standards. Nat Commun 2016;7:12150.
32. Novitskiy S V, Pickup M W, Gorska A E, Owens P, Chytil A, Aakre M, et al. TGF-beta receptor II loss promotes mammary carcinoma progression by Th17 dependent mechanisms. Cancer Discov 2011;1(5):430-41.
33. Schafer C C, Wang Y, Hough K P, Sawant A, Grant S C, Thannickal V J, et al. Indoleamine 2,3-dioxygenase regulates anti-tumor immunity in lung cancer by metabolic reprogramming of immune cells in the tumor microenvironment. Oncotarget 2016;7(46): 75407-24.
34. Yu J, Wang Y, Yan F, Zhang P, Li H, Zhao H, et al. Noncanonical NF-kappaB activation mediates STAT3-stimulated IDO upregulation in myeloid-derived suppressor cells in breast cancer. J Immunol 2014;193(5):2574-86.
35. Van de Velde L A, Gingras S, Pelletier S, Murray P J. Issues with the Specificity of Immunological Reagents for Murine IDO1. Cell Metab 2016;23(3):389-90.
36. Thomas S, DuHadaway J, Prendergast G C, Laury-Kleintop L. Specific in situ detection of murine indoleamine 2,3-dioxygenase. J Cell Biochem 2014;115(2):391-6.
37. Zhao T, Ding X, Du H, Yan C. Myeloid-derived suppressor cells are involved in lysosomal acid lipase deficiency-induced endothelial cell dysfunctions. J Immunol 2014;193(4):1942- 53.
38. Fainaru O, Hantisteanu S, Hallak M. Immature myeloid cells accumulate in mouse placenta and promote angiogenesis. Am J Obstet Gynecol 2011;204(6):544 e18-23.
39. Kammertoens T, Friese C, Anna A, Idel C, Briesemeister D, Rothe M, et al. Tumour ischaemia by interferon-gamma resembles physiological blood vessel regression. Nature 2017;545(7652):98-102.
40. Deng J, Liu X, Rong L, Ni C, Li X, Yang W, et al. IFN-responsiveness of endothelial cells leads to efficient angiostasis in tumours involving down-regulation of Dll4. J Pathol 2014;233:170-82.
41. Gopinathan G, Milagre C, Pearce O M, Reynolds L E, Hodivala-Dilke K, Leinster D A, et al. Interleukin-6 Stimulates Defective Angiogenesis. Cancer Res 2015;75(15):3098-107.
42. Bunt S K, Yang L, Sinha P, Clements V K, Leips J, Ostrand-Rosenberg S. Reduced inflammation in the tumor microenvironment delays the accumulation of myeloid-derived suppressor cells and limits tumor progression. Cancer Res 2007;67(20):10019-26.
43. Middleton K, Jones J, Lwin Z, Coward J I. Interleukin-6: an angiogenic target in solid tumours. Crit Rev Oncol Hematol 2014;89(1):129-39.
44. Condamine T, Ramachandran I, Youn J I, Gabrilovich D I. Regulation of tumor metastasis by myeloid-derived suppressor cells. Annu Rev Med 2015;66:97-110.
45. Hoffmann D, Dvorakova T, Stroobant V, Bouzin C, Daumerie A, Solvay M, et al. Tryptophan 2,3-Dioxygenase Expression Identified in Human Hepatocellular Carcinoma Cells and in Intratumoral Pericytes of Most Cancers. Cancer Immunol Res 2020;8(1):19-31.
46. Tian L, Goldstein A, Wang H, Ching Lo H, Sun Kim I, Welte T, et al. Mutual regulation of tumour vessel normalization and immunostimulatory reprogramming. Nature 2017;544(7649):250-54.
47. Jain R K. Normalization of tumor vasculature: an emerging concept in antiangiogenic therapy. Science 2005;307(5706):58-62.

What is claimed is:

1. A method of treating a retinopathy or inhibiting pathologic neovascularization in a subject, the method comprising ablating or inhibiting IDO-dependent vascularizing cells (IDVCs) in the eye of the subject.

2. The method of claim 1, wherein said IDVCs are functionally characterized as having a role in neovascularization.

3. The method of claim 1 or 2, wherein the establishment and maintenance of neovascularization requires the induction of IDO1 within the IDVCs.

4. The method of any of claims 1 to 3 wherein the inhibition of IDVC activity is achieved by inhibition of IDO1.

5. The method of any of claims 1 to 4, wherein said IDVCs are inhibited or ablated using an antibody directed to a cell-surface marker of the IDVCs.

6. The method of claim 5, wherein the cell surface marker is selected from CD33, CD11b, CD15, and CD66.

7. The method of claim 5, wherein the cell surface marker is selected from CD274 (PDL1), MHC class II, CD4, CD31 (PECAM-1), CD202B (TIE2), CD205 (DEC-205), Siglec 8, and EMR1

8. The method any of claims 1 to 7, the method comprising blocking or inhibiting the Integrated Stress Response (ISR) in IDVCs.

9. The method any of claims 1 to 7, the method comprising blocking or inhibiting the Integrated Stress Response (ISR) in ocular tissues.

10. The method any of claims 1 to 7, the method comprising blocking or inhibiting the Integrated Stress Response (ISR) in the retina or choroid.

11. The method of any of claims 8 -10, comprising blocking or inhibiting the expression, induction, activity, and/or signaling of an ISR pathway node.

12. The method of claim 11, comprising:
(a) blocking or inhibiting the expression, induction, activity, or signaling of GCN2; and/or
(b) blocking or inhibiting the expression, induction, activity, or signaling of ATF4; and/or
(c) blocking or inhibiting the expression, induction, activity, or signaling of CHOP.

13. The method of any of claims 1 to 12, further comprising blocking or inhibiting IL-6.

14. A method of treating retinopathy or inhibiting pathologic neovascularization in a subject, the method comprising blocking or inhibiting signaling molecules downstream of the Integrated Stress Response.

15. The method of claim 14, wherein said signaling molecule is a cytokine.

16. The method of claim 115, wherein said cytokine is IL-6.

17. The method of any of claims 1 to 16, further comprising blocking or inhibiting the expression, induction, activity, or signaling of any form of vascular endothelial growth factor (VEGF).

18. The method according to claim 17, wherein the VEGF inhibitor is administered less frequently than it would be administered as a sole therapeutic agent.

19. The method according to any preceding claim, further comprising administering in combination an inhibitor of the expression, induction, activity, or signaling of indoleamine 2,3 dioxygenase-1 (IDO1).

20. The method according to any preceding claim, wherein the IDVCs are located in the retina of the eye.

21. The method according to any preceding claim, wherein the IDVCs are located in the choroid of the eye.

22. The method according to any preceding claim, wherein the route of administration of a therapeutic agent comprises oral administration, intravenous injection, intra-nasal administration, sublingual administration, intravitreal injection, intra-ocular injection, administration via a depot formulation or device, or administration via eye drops.

23. The method according to any preceding claim, wherein the blocker or inhibitor of IDO-1 comprises at least one of:
i. 1-methyl-D-tryptophan (indoximod), 1-methyl-L-tryptophan, a racemic mixture of 1-methyl-D-tryptophan and 1-methyl-L-tryptophan, epacadostat, navoximod (GDC-0919), and NLG802, or a salt, enantiomer or pro-drug thereof;
ii. 1-R-D-tryptophan or 1-R-L-tryptophan, wherein R is a C1-C12 alkyl;
iii. methylthiohydantoin-DL-tryptophan (MTH-Trp), β-(3-β)-DL-alanine, β-(3-benzo(b)thienyl)-DL-alanine, 6-nitro-L-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-DL-tryptophan, 5-bromoindoxyl diacetate, Naphthoquinone-based, S-allyl-brassinin, S-benzyl-brassinin, 5-Bromo-brassinin, Phenylimidazole-based, 4-phenylimidazole, Exiguamine A, and NSC401366; or
iv. BMS-986205/ONO-7701, PF-06840003/EOS200271, MK-7162/IOM2983, LY3381916, KHK2455, HTI-1090/SHR9146, DN1406131, RG70099, Roxyl-WL, TPST-8844, Ethyl pyruvate, Amg-1 or DX-03-12, or a salt, enantiomer or pro-drug or any therapeutically effective formulation thereof.

24. The method according to claim 17 or 18, wherein the VEGF inhibitor comprises one or more of: ranibizumab (Lucentis®), bevacizumab (Avastin®), aflibercept (Eylea®), brolucizumab (Boevu®), pegaptanib (Macugen®), Abicipar pegol, the ranibizumab biosimilars FYB201, PF582, SB11, and Xlucane, the aflibercept biosimilar MYL-1701P/M-710, or conbercept, faricimab/RG7716 (bispecific antibody VEGF-A+Ang-2), OPT-302 (VEGF-C/D ‘trap’), KS301 (Kodiak Sciences—anti-VEGF polymer conjugated biologic), KS501 (Kodiak Sciences—anti-VEGF trap plus anti-IL6 Antibody Fusion).

25. The method according to claim 11 or 12, wherein the ISR pathway node is inhibited by a small molecule that inhibits the translation or transcription of said ISR pathway node.

26. The method according to claim 11 or 12, wherein the ISR pathway node is inhibited by a biologic molecule that inhibits the translation or transcription of said ISR pathway node.

27. The method according to any one of claim 26 wherein the biologic molecule is an antibody or fragment thereof.

28. The method according to any one of claim 11 or 12, wherein the ISR pathway node is inhibited by a nucleic acid molecule that inhibits the translation or transcription of said ISR pathway node.

29. The method according to claim 28, wherein said nucleic acid molecule is an siRNA or shRNA.

30. The method of any preceding claim comprising administering a GCN2 inhibitor.

31. The method of claim 30, wherein the GCN2 inhibitor is selected from GCN2-IN-1 (A-92), GCN2iA, GZD824, inhibitors based on a triazolo[4,5-d]pyrimidine scaffold.

32. The method of any preceding claim comprising administering a CHOP inhibitor.

33. The method of any preceding claim comprising administering an ATF4 inhibitor.

34. The method of claim 33, wherein the ATF4 inhibitor is selected from ursolic acid, tomatidine, GSK2606414, and TRIB3.

35. The method according to claim 25, wherein the ISR pathway-blocking drug is selected from: GSK-2606414, RPT-GCN2i, AMG-PERK44, and trans-ISRIB.
